SEQUENCE LISTING (37 sequences)

SEQ ID NO: 1 - protein, 343 aa, Artificial Sequence
  Synthetic Polypeptide
GPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 2 - protein, 10 aa, Bos taurus
LRELHLNNNC

SEQ ID NO: 3 - protein, 10 aa, Homo sapiens
LRELHLDNNC

SEQ ID NO: 4 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 5 - protein, 205 aa, Homo sapiens
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 6 - protein, 196 aa, Homo sapiens
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 7 - protein, 222 aa, Homo sapiens
CQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLC

SEQ ID NO: 8 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 9 - protein, 30 aa, Homo sapiens
RRRPKGRGKRRREKQRPTDCHLCGDAVPRR

SEQ ID NO: 10 - protein, 22 aa, Homo sapiens
RRRPKGRGKRRREKQRPTDCHL

SEQ ID NO: 11 - protein, 18 aa, Homo sapiens
RRPKGRGKRRREKQRPTD

SEQ ID NO: 12 - protein, 15 aa, Homo sapiens
RRRPKGRGKRRREKQ

SEQ ID NO: 13 - protein, 8 aa, Homo sapiens
GKRRREKQ

SEQ ID NO: 14 - protein, 8 aa, Homo sapiens
RRRPKGRG

SEQ ID NO: 15 - protein, 21 aa, Homo sapiens
RRKTKGKRKRSRNSQTEEPHP

SEQ ID NO: 16 - protein, 98 aa, Homo sapiens
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 17 - protein, 30 aa, Homo sapiens
GRREEKVGKKEKIGKKKRQKKRKAAQKRKN

SEQ ID NO: 18 - RNA, 19 nt, Artificial Sequence
  Synthetic Polynucleotide
uaaucguucaguuuucugg

SEQ ID NO: 19 - RNA, 19 nt, Artificial Sequence
  Synthetic Polynucleotide
aaaucuuuaaggacagggc

SEQ ID NO: 20 - RNA, 19 nt, Artificial Sequence
  Synthetic Polynucleotide
gaguauuuaaccacagucg

SEQ ID NO: 21 - RNA, 19 nt, Artificial Sequence
  Synthetic Polynucleotide
aaacucacaagcuuucucg

SEQ ID NO: 22 - DNA, 19 bp, Artificial Sequence
  Synthetic Polynucleotide
taatcgttcagttttctgg

SEQ ID NO: 23 - DNA, 19 bp, Artificial Sequence
  Synthetic Polynucleotide
aaatctttaaggacagggc

SEQ ID NO: 24 - DNA, 19 bp, Artificial Sequence
  Synthetic Polynucleotide
gagtatttaaccacagtcg

SEQ ID NO: 25 - DNA, 19 bp, Artificial Sequence
  Synthetic Polynucleotide
aaactcacaagctttctcg

SEQ ID NO: 26 - RNA, 19 nt, Artificial Sequence
  Synthetic Polynucleotide
cuggaaaauucagugauga

SEQ ID NO: 27 - RNA, 19 nt, Artificial Sequence
  Synthetic Polynucleotide
cccuggauuuugaagaaca

SEQ ID NO: 28 - RNA, 19 nt, Artificial Sequence
  Synthetic Polynucleotide
ccaucgaguacauaaaaca

SEQ ID NO: 29 - RNA, 19 nt, Artificial Sequence
  Synthetic Polynucleotide
ccagaaaacugaacgauua

SEQ ID NO: 30 - RNA, 19 nt, Artificial Sequence
  Synthetic Polynucleotide
gcccuguccuuaaagauuu

SEQ ID NO: 31 - RNA, 19 nt, Artificial Sequence
  Synthetic Polynucleotide
cgacugugguuaaauacuc

SEQ ID NO: 32 - RNA, 19 nt, Artificial Sequence
  Synthetic Polynucleotide
cgagaaagcuugugaguuu

SEQ ID NO: 33 - DNA, 19 bp, Artificial Sequence
  Synthetic Polynucleotide
ccagaaaactgaacgatta

SEQ ID NO: 34 - DNA, 19 bp, Artificial Sequence
  Synthetic Polynucleotide
gccctgtccttaaagattt

SEQ ID NO: 35 - DNA, 19 bp, Artificial Sequence
  Synthetic Polynucleotide
cgactgtggttaaatactc

SEQ ID NO: 36 - DNA, 19 bp, Artificial Sequence
  Synthetic Polynucleotide
cgagaaagcttgtgagttt

SEQ ID NO: 37 - RNA, 19 nt, Artificial Sequence
  Synthetic Polynucleotide
ucaucacugaauuuuccag

(19) United States
(12) Patent Application Publication
(10) Pub. No.: US 2025/0319117 A1
(43) Pub. Date: Oct. 16, 2025

(54) METHODS AND COMPOSITIONS FOR TREATING FIBROSIS

(71) Applicant: THE UNIVERSITY OF CHICAGO, Chicago, IL (US)

(72) Inventors: Jeffrey HUBBELL, Chicago, IL (US); Michael WHITE, Chicago, IL (US)

(73) Assignee: THE UNIVERSITY OF CHICAGO, Chicago, IL (US)

(21) Appl. No.: 18/566,264

(22) PCT Filed: Jun. 2, 2022
(86) PCT No.: PCT/US2022/072720

§ 371 Date: Dec. 1, 2023

Related U.S. Application Data
(60) Provisional application No. 63/196,594, filed on Jun. 3, 2021.

(51) Int. Cl. A61K 31/713; A61K 38/00; A61K 39/00; A61K 47/68; A61P 11/00; C07K 14/78; C07K 16/22; C07K 16/28; C12N 15/113

(52) U.S. Cl. CPC A61K 31/713; A61K 47/6811; A61K 47/6849; A61P 11/00; C07K 14/78; C07K 16/22; C07K 16/2845; C12N 15/113; A61K 38/00; A61K 2039/505; C07K 2317/70; C12N 2310/14

(57) ABSTRACT

Blocking antibodies against αMβ2 (CBP-α-αMβ2), αM (CBP-α-αM), α3 (CBP-α-α3), and anti-TGFβ as well as inhibitors of Talin2 reverse fibrosis in a mouse fibrosis model. Accordingly, aspects of the disclosure relate to inhibitors of Talin2 and inhibitors of the integrins αMβ2, αM, and α3. Aspects relate to a method for treating and/or reversing fibrosis in a subject comprising administering a composition comprising an inhibitor of Talin2 or a composition comprising a nucleic acid of the disclosure. Further aspects relate to a method for treating and/or reversing fibrosis in a subject comprising administering a composition comprising an antibody conjugate of the disclosure or a composition comprising an inhibitor or blocking agent of integrin α3, αM, αMβ2, or combinations thereof to the subject. Methods also include treating kidney fibrosis in a subject comprising administering a composition comprising an anti-TGFβ antibody operatively linked to an ECM-affinity peptide. The methods may be for reducing or decreasing the amount of existing fibrosis. The methods differ from traditional methods for treating fibrosis, since the current methods do not delay or inhibit the progression of fibrosis, but instead have shown to reverse, reduce, and/or decrease existing fibrosis. Accordingly, methods of the disclosure may be used in a manner that provides treatment to existing fibrosis rather than a prophylactic to prevent more fibrosis.

### CROSS-REFERENCE TO RELATED APPLICATIONS

This application is a national phase application under 35 U.S.C. § 371 of International Application No. PCT/US2022/072720, filed Jun. 2, 2022, which claims the benefit of priority of U.S. Provisional Patent Application No. 63/196,594 filed Jun. 3, 2021, each of which are hereby incorporated by reference in their entirety.

### SEQUENCE LISTING

The instant application contains a Sequence Listing which has been submitted in ASCII format and is hereby incorporated by reference in its entirety. Said ASCII copy, created on Sep. 18, 2024, is named ARCDP0730WO_SUB.txt and is 42,167 bytes in size.

### BACKGROUND OF THE INVENTION

### I. Field of the Invention

This invention relates to the field of treatment of fibrotic diseases.

### II. Background

Fibrosing diseases—including pulmonary fibrosis, congestive heart failure, liver cirrhosis, and end-stage kidney disease—are involved in 45% of deaths in the United States [1, 2]. There are only 2 FDA approved treatments for fibrosis, pirfenidone and nintedanib [2, 3]. Pirfenidone and nintedanib slow, but do not reverse, the progression of fibrosis [4], with mechanisms of action that are poorly understood [5].

A major goal of research in fibrosis is developing a treatment capable of reversing established fibrosis [1]. However, there is only one treatment that has, thus far, shown even a modest ability to reverse even some symptoms of fibrosis in some patients, recombinant pentraxin-2 [6]. To fully reverse fibrosis, collagen deposition would need to cease, existing ECM would need to be remodeled, and myofibroblasts would need to de-activate to reduce the stiffness of the tissue. Interrupting collagen deposition alone can destabilize scar tissue [1] and monocyte-derived cells are capable of removing deposited ECM while regenerating tissue [7].

There is a need in the art for treatments that reverse existing fibrosis, rather than just slow the progression.

### SUMMARY OF THE INVENTION

Here the inventors demonstrate that blocking antibodies against αMβ2 (CBP-α-αMβ2), αM (CBP-α-αM), and α3 (CBP-α-α3) as well as inhibitors of Talin2 reverse fibrosis in a mouse fibrosis model. Accordingly, aspects of the disclosure relate to inhibitors of Talin2 and inhibitors of the integrins αMβ2, αM, and α3. Aspects relate to a method for treating and/or reversing fibrosis in a subject comprising administering a composition comprising an inhibitor of Talin2 or a composition comprising a nucleic acid of the disclosure. Further aspects relate to a method for treating and/or reversing fibrosis in a subject comprising administering a composition comprising an antibody conjugate of the disclosure or a composition comprising an inhibitor or blocking agent of integrin α3, αM, αMβ2, or combinations thereof to the subject. Methods also include treating kidney fibrosis in a subject comprising administering a composition comprising an anti-TGFβ antibody operatively linked to an ECM-affinity peptide. The antibody may be the XT3.11 monoclonal anti-TGFβ antibody. In some aspects, the inhibitor or antibody conjugate comprises a heavy chain variable region comprising CDR1, CDR2, and CDR3 from the heavy chain variable region of the XT3.11 anti-TGFβ antibody and a light chain variable region comprising CDR1, CDR2, and CDR3 from the light chain variable region of the XT3.11 anti-TGFβ antibody. The methods may be for reducing or decreasing the amount of existing fibrosis. The methods differ from traditional methods for treating fibrosis, since the current methods do not delay or inhibit the progression of fibrosis, but instead have shown to reverse, reduce, and/or decrease existing fibrosis. Accordingly, methods of the disclosure may be used in a manner that provides treatment to existing fibrosis rather than a prophylactic to prevent more fibrosis. Methods of the disclosure relate to reducing existing fibrosis in a subject having fibrosis. Methods also relate to reversing fibrosis in a subject having fibrosis.

Aspects provide for a nucleic acid having a sequence that is at least 80% sequence identity to one of SEQ ID NOS:18-25. Aspects provide for a nucleic acid having a sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS:18-25. Aspects provide for a nucleic acid having a sequence with 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS:18-25. Also provided are cDNAs of or encoding nucleic acids of the disclosure, expression vectors comprising cDNAs or nucleic acids of the disclosure, and host cells comprising a nucleic acid, expression vector, or cDNA of the disclosure. The host cell may be a bacterial or mammalian cell. In certain aspects, the host cell is a human cell. Also described is a method for making the nucleic acid of the disclosure comprising expressing the nucleic acid or expression vector of the disclosure in a cell and isolating the expressed RNA or transferring the nucleic acid of the disclosure into a host cell and isolating replicated nucleic acids.

Further aspects relate to an antibody conjugate comprising an integrin α3, αM, or αMβ2 antibody operatively linked to an extracellular matrix (ECM)-affinity peptide. Also described are one or more nucleic acids that encode the antibody conjugates of the disclosure, expression vectors comprising nucleic acids of the disclosure, and host cells comprising the nucleic acids or expression vectors of the disclosure. Further aspects relate to a method for making an antibody conjugate comprising expressing one or more nucleic acids or expression vectors of the disclosure in a cell and isolating the expressed protein. Methods also include a method for making an antibody conjugate comprising conjugating one or more ECM peptides to an antibody. Aspects also relate to compositions comprising nucleic acids, cDNAs, host cells, expression vectors, or antibody conjugates of the disclosure.

The nucleic acid of the disclosure may be a modified nucleic acid. The nucleic acid may comprise at least one locked nucleic acid residue and/or at least one phosphorothioate linkage. The nucleic acid may also comprise an ethylene bridged nucleotide, a peptide nucleic acid, a phosphorodiamidate morpholino, a 5′-Vinyl-phosphonate, a 2′O-methyl, 2′F, or combinations thereof. The nucleic acid may be RNA or DNA. The nucleic acid may be double stranded or single-stranded. In some aspects, the nucleic acid is double stranded. In some aspects, the nucleic acid is a double stranded RNA molecule.

In some aspects, nucleotides 1 and/or 2 are modified with a 2′O-methyl. In some aspects, the nucleic acid is a RNA molecule, and all C and all U nucleotides are modified with a 2′O-methyl. The nucleic acid may comprise a sense strand and an antisense strand. In some aspects, the antisense strand comprises a sequence having at least 80% sequence identity to one of SEQ ID NOS:18-25 and the sense strand comprises a sequence that is complementary to the antisense strand or at least partially complementary to the sense strand. In some aspects, the antisense strand comprises a sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS:18-25 (or any range derivable therein) and the sense strand comprises a sequence that is complementary to the antisense strand or at least partially complementary to the sense strand. In some aspects, nucleotide 1, nucleotide 2, all C and/or all U nucleotides on the sense strand are 2′O-methyl modified. In some aspects, nucleotides 1 and 2 and all C and all U nucleotides on the sense strand are 2′O-methyl modified. In some aspects, all C nucleotides and all U nucleotides on the antisense strand are 2′F modified. The sense strand may be nineteen nucleotides in length. In some aspects, the sense strand may be at least, may be at most, or may be exactly 10, 11, 12, 13, 14, 15, 16, 17, 18, 19, 20, 21, 22, 23, 24, 25, or 26 nucleotides in length, or any range derivable therein. In some aspects, the antisense strand is 21 nucleotides in length. In some aspects, the antisense strand may be at least, may be at most, or may be exactly 10, 11, 12, 13, 14, 15, 16, 17, 18, 19, 20, 21, 22, 23, 24, 25, or 26 nucleotides in length, or any range derivable therein. The sense strand and the antisense strand may form a duplex having a two nucleotide overhang at the 3′ end of the antisense strand, said two nucleotide overhang comprising phosphorothioate linkages. The nucleic acid may comprise or further comprise a cholesterol molecule attached to the 3′ end of the nucleic acid via a C5 linker molecule thereby forming a cholesterol-linker-sense strand structure of:

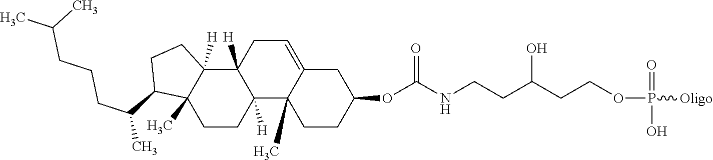

The cholesterol molecule may be attached to the 3′ or 5′ end of the sense strand. In certain aspects, the cholesterol molecule is attached to the 3′ end of the sense strand. The nucleic acid may comprise a phosphate group at the 5′ end of the antisense strand. The nucleic acid may comprise three mismatches on the sense strand with the corresponding nucleotides on the antisense strand, wherein the mismatches are between nucleotide 6 on the sense strand and opposite nucleotide 14 on the antisense strand, nucleotide 13 on the sense strand and opposite nucleotide 7 on the antisense strand, and nucleotide 19 on the sense strand and opposite nucleotide 1 on the antisense strand; wherein each nucleotide number refers to the nucleotide's position in an identified strand as counted form the identified strand's 5′ end, and at all positions other than positions 6, 13 and 19 on the sense strand, there is a nucleotide that is complementary to the nucleotide on the opposite strand. In some aspects, at least one of the mismatches is a G across from an A. In some aspects, at least one of the mismatches is an A across from a C. In some aspects, at least one of the mismatches is an A across from an A. In some aspects, at least one of the mismatches is a G across from a G. In some aspects, at least one of the mismatches is a C across from a C. In some aspects, at least one of the mismatches is a U across from a U. In some aspects, the overhang is UU.

The subject may be one that has and/or has been diagnosed with fibrosis. The subject may be one that has been previously treated for fibrosis. In some aspects, the subject does not have a kidney disease or acute kidney injury. In some aspects, the subject does not have chronic kidney disease. In some aspects, the inhibitor described in the methods of the disclosure is an antibody. Antibodies suitable for use in the methods of the disclosure are known and described in the art. For example, blocking antibodies for anti-cd11b include antibody clones MEM-170, M1-70, CBRM1-5, ICRF44, hCD11b, MAB0813, mAb107, CBDH339, H5A4, and mCD11b. Blocking antibodies for anti-ITGA3 include P1B5, CBDH1356, and OX-81. These antibodies are available from commercial providers. In addition, other blocking antibodies useful in the methods of the disclosure can be found by searching the database antibodypedia on the world wide web. The antibody may comprise the anti-αMβ2 CBRM1/5 antibody or the anti-α3 3F9G4 antibody. The inhibitor may be administered systemically. In some aspects, the method comprises systemic administration of an inhibitory or blocking antibody. In some aspects, the inhibitor inhibits the activated form of integrin αMβ2. In some aspects, the inhibitor is linked to an extracellular matrix (ECM)-affinity peptide. In aspects of the disclosure the inhibitor or the antibody conjugate may comprise a humanized version of the CBRM1/5 or 3F9G4 antibodies. In some aspects, the inhibitor or antibody conjugate comprises a heavy chain variable region comprising CDR1, CDR2, and CDR3 from the heavy chain variable region of the CBRM1/5 antibody and a light chain variable region comprising CDR1, CDR2, and CDR3 from the light chain variable region of the CBRM1/5 antibody. In some aspects, the inhibitor or antibody conjugate comprises a heavy chain variable region comprising CDR1, CDR2, and CDR3 from the heavy chain variable region of the 3F9G4 antibody and a light chain variable region comprising CDR1, CDR2, and CDR3 from the light chain variable region of the 3F9G4 antibody. Further aspects relate to an antibody or antigen binding fragment comprising a heavy chain variable region and a light chain variable region, wherein the heavy chain variable region comprises a HCDR1, HCDR2, and HCDR3 having at least 80% sequence identity or at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the HCDR1, HCDR2, and HCDR3 from the heavy chain variable region of CBRM1/5 and wherein the light chain variable region comprises a LCDR1, LCDR2, and LCDR3 having at least 80% sequence identity or at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of CBRM1/5. Further aspects relate to an antibody or antigen binding fragment comprising a heavy chain variable region and a light chain variable region, wherein the heavy chain variable region comprises a HCDR1, HCDR2, and HCDR3 having at least 80% sequence identity or at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the HCDR1, HCDR2, and HCDR3 from the heavy chain variable region of 3F9G4 and wherein the light chain variable region comprises a LCDR1, LCDR2, and LCDR3 having at least 80% sequence identity or at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of 3F9G4. The CDR may be one that has been determined by Kabat, IMGT, or Chothia. In further aspects, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

The antibody conjugate of the disclosure may be an anti-αMβ2 antibody. In some aspects, the antibody is the CBRM1/5 antibody clone. In some aspects, the antibody is the 3F9G4 antibody clone. The antibody may be one that specifically binds to the activated form of integrin αMβ2. The structure of the activated form of αMβ2 is known in the art and described in Oxvig et al., Proc. Natl. Acad. Sci. USA, Vol. 96, pp. 2215-2220, March 1999, which is herein incorporated by reference. Furthermore, antibodies that bind to the active form, such as the CBRM1/5 clone are known in the art.

The ECM-affinity peptide comprises a decorin peptide. The decorin peptide may comprise SEQ ID NO:1 or a peptide with at least 85% sequence identity to SEQ ID NO:1. In some aspects, the decorin peptide may comprises a peptide having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:1. The decorin peptide may also comprise SEQ ID NO:2 or SEQ ID NO:3, or comprises a peptide with at least 85% sequence identity to SEQ ID NO:2 or SEQ ID NO:3. In some aspects, the decorin peptide comprises a peptide with at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to SEQ ID NO:2 or SEQ ID NO:3. The ECM-affinity peptide may comprise a peptide from placenta growth factor-2 (PlGF-2). The peptide may comprise SEQ ID NO:10 or a sequence with at least 85% sequence identity to SEQ ID NO:10. In some aspects, the peptide comprises a sequence with or with at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:10 (or any range derivable therein). The peptide may comprise a von Willebrand factor (VWF) peptide. The VWF peptide may be a VWF A1 or A3 peptide. The VWF peptide may comprise SEQ ID NO:5, SEQ ID NO:7, fragments thereof, or a peptide that has at least 85% sequence identity to SEQ ID NO:5, SEQ ID NO:17, or fragments thereof. In some aspects, the VWF peptide may comprise a peptide that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to SEQ ID NO:5, SEQ ID NO:17, or fragments thereof. The VWF peptide may comprise SEQ ID NO:6 or a peptide comprising at least 85% sequence identity to SEQ ID NO:6. In some aspects, the VWF peptide may comprise a peptide comprising at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to SEQ ID NO:6. The peptide may comprises a CXCL-12 peptide. The CXCL-12 peptide may comprise a CXCL-127 peptide. The peptide may comprise SEQ ID NO:17 or a peptide with at least 85% sequence identity to SEQ ID NO:17. In some aspects, the peptide comprises a peptide with at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to SEQ ID NO:17.

In some aspects, the ratio of peptide to antibody is about 1:1 to 10:1. In some aspects, the ratio of peptide to antibody is at least, at most, or is about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 30:1, 35:1, 40:1, 45:1, 50:1, or 100:1 (or any derivable range therein).

The peptide may be covalently linked to the antibody. In some aspects, the peptide is crosslinked to the antibody through a bifunctional linker. Linkers, such as amino acid or peptidimimetic sequences may be inserted between the peptide and/or antibody sequence. In an aspect, a fynomer domain is joined to a Heavy (H) chain or Light (L) chain immediately after the last amino acid at the amino(NH₂)-terminus or the carboxy(C)-terminus of the Heavy (H) chain or the Light (L) chain. Linkers may have one or more properties that include a flexible conformation, an inability to form an ordered secondary structure or a hydrophobic or charged character which could promote or interact with either domain. Examples of amino acids typically found in flexible protein regions may include Gly, Asn and Ser. Other near neutral amino acids, such as Thr and Ala, may also be used in the linker sequence. The length of the linker sequence may vary without significantly affecting the function or activity of the fusion protein (see, e.g., U.S. Pat. No. 6,087,329). In a particular aspect, a peptide and an antibody heavy or light chain are joined by a peptide sequence having from about 1 to 25 amino acid residues. Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as C_{N} (where N=1-100 carbon atoms, e.g., C, CC, CCC, CCCC, CCCCC, CCCCCC, CCCCCCC, CCCCCCCC). In some aspects, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some aspects, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus). Further, the linker may be maleimidocaproyl (mc).

In methods of the disclosure, the inhibitor may be a nucleic acid inhibitor. In some aspects, the inhibitor comprises a small interfering RNA (siRNA), micro RNA (miRNA), short hairpin RNA (shRNA), or an antisense oligonucleotide (ASO). The inhibitor may comprise one of SEQ ID NOS:18-25. In some aspects, the inhibitor comprises a sequence having at least or exactly 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to one of SEQ ID NOS:18-25.

The fibrosis may comprise dermal, heart, renal, liver, or pulmonary fibrosis. In some aspects, the fibrosis excludes dermal, heart, renal, liver, or pulmonary fibrosis. In some aspects, the fibrosis comprises dermal fibrosis. The administration may comprise topical administration to the skin fibrosis. In some aspects, the fibrosis comprises pulmonary fibrosis. The pulmonary fibrosis may comprise drug-induced, radiation-induced, environmental, autoimmune, occupational, or idiopathic pulmonary fibrosis. In some aspects, the pulmonary fibrosis may exclude drug-induced, radiation-induced, environmental, autoimmune, occupational, or idiopathic pulmonary fibrosis. The fibrosis may be fibrosis associated with non-alcoholic fatty liver disease (NAFLD). The fibrosis may also be fibrosis associated with nonalcoholic steatohepatitis (NASH). The subject may be one that has or has been diagnosed with NAFLD and/or NASH. In certain aspects NAFLD and/or NASH is excluded in the claimed methods. The methods of the disclosure may also exclude the treatment of NASH and/or NAFLD and/or subjects diagnosed with NASH or NAFLD.

The composition may be administered by inhalation or intranasally. The composition may comprise a suspension. The suspension may comprise the inhibitor in powdered form as a pharmaceutical carrier. The inhibitor may comprise a lyophilized powder. In some aspects, the inhibitor and the pharmaceutical carrier are mixed just prior to administration. In some aspects, the nucleic acid inhibitor targets a non-coding region of the Talin2 gene. In some aspects, the nucleic acid inhibitor targets an untranslated region or the open reading frame of the Talin2 gene.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

The subject may be a human, mouse, pig, cow, sheep, rabbit, or rat. In some aspects, the subject is a non-human primate. In some aspects, the subject is a human or a mammal.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment and/or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D: Antibodies against integrins α3, αM, and αMβ2 de-differentiate human myofibroblasts from monocyte precursors. Freshly isolated human monocytes (A-D) were differentiated into myofibroblasts and subsequently treated with (A) α-αMβ2, α-αM, α-α3, or α-α2β1 antibodies or (B) leukadherin at the indicated concentrations over 1 week, and the number of remaining myofibroblasts was assessed by morphology. Monocyte-derived myofibroblasts were treated with antibodies at 500 ng/ml and leukadherin at 2 ng/ml and analyzed via flow cytometry for (C) αSMA⁺ collagen I⁺ double positive cells and (D) talin2⁺. n ranges from 3 to 15. *=statistical significance of P<0.05, <0.01, or <0.001, significance vs isotype control antibody, (A and B) 2-way ANOVA (with Fisher's LSD post test), (C and D)

FIG. 2A-D: Antibodies against integrins α3, αM, and αMβ2 de-differentiate mouse myofibroblasts from monocyte precursors. Freshly isolated mouse monocytes (A-D) were induced to become myofibroblasts with IL-13, MCSF, and β-mercaptoethanol, treated with (A) α-αMβ2, α-αM, α-α3, or α-α2β1 antibodies or (B) leukadherin at the indicated concentrations over 1 week, and the number of myofibroblasts was assessed by morphology. Monocyte-derived myofibroblasts were treated with antibodies at 500 ng/ml and leukadherin at 2 ng/ml, allowed to de-differentiate, and analyzed via flow cytometry for (C) αSMA⁺ and collagen I⁺ double positive cells and (D) talin2⁺. n ranges from 2 to 7. *=statistical significance of P<0.05, <0.01, or <0.001, significance vs isotype control antibody, (A and B) 2-way ANOVA (with Fisher's LSD post test), (C and D) Student's t-test.

FIG. 3A-E: Conjugation of decorin's collagen-binding peptide (CBP) to anti-integrin-αM (α-αM) increases antibody concentration in fibrotic lungs. Mice were intranasally instilled with 75 g of bleomycin sulfate (fibrotic) or PBS (healthy) and injected 1 week later with Cy7-α-αM or Cy7-CBP-α-αM. (A) Heart, lung, spleen, kidneys, and liver were harvested 48 hr after injection, and fluorescence intensity was measured via IVIS. (B) The number of photons from lungs or (C) spleen. Fluorescence of the resected organs was pooled and the percentage of fluorescence associated with (D) lungs and (E) spleen. n ranges from 2 to 4. *=statistical significance of P<0.05, <0.01, or <0.001, significance is fluorescence of fibrotic lungs vs fluorescence of healthy lungs, Student's t-test.

FIG. 4A-F: Antibodies against integrins α3, αM, and αMβ2 rescues the fibrotic damage from bleomycin insult to mouse lungs. 50 μg antibody was injected i.v. after 7, 9, 11, 14, 16, and 18 days following insult by 75 g bleomycin. (A-C) mouse weights after treatment with (A) α-α3 and CBP-α-α3, (B) α-αM and CBP-α-αM, (C) α-αMβ2 and CBP-α-αMβ2. (D) Collagen content from the right, multi-lobed lung assessed by hydroxyproline assay. (E) Data from D divided by dry weight of right lobes of mouse lungs. (F) Blinded Ashcroft scoring. n ranges from 8 to 9. *=statistical significance of P<0.05, <0.01, or <0.001, significance vs fibrotic lungs (A-C) 2-way ANOVA (with Fisher's LSD post-test), (D-F) Student's t-test.

FIG. 5A-H: Representative images of left, single-lobed lungs stained with Massons's trichrome. (A) Uninsulted lungs instilled with PBS. Lungs insulted with (B-H) 75 μg bleomycin, and instilled with (C) α-α3, (D) CBP-α-α3, (E) α-αM, (F) CBP-α-αM, (G) α-αMβ2, (F) CBP-α-αMβ2. Lungs were harvested 3 weeks after insult and stained via Masson's trichrome. Scores in FIGS. 4, 6, and inset images in FIG. 14.

FIG. 6A-F: Antibodies against integrins α3, αM, and αMβ2 rescue the fibrotic damage from both male and female mice. Data from FIG. 4, treatment by α-α3, CBP-α-α3, α-αM, CBP-α-αM, α-αMβ2, CBP-α-αMβ2 compared across male (n=5 or 6, A, C, E, G) and female (n=3, B, D, F, H) mice for amount of collagen in lungs (A, B), Collagen as a percentage of dry lung weight (C, D), Ashcroft scores (E, F), and blinded Ashcroft scores (E, F). n ranges from 3 to 6. *=statistical significance of P<0.05, <0.01, or <0.001, significance vs fibrotic lungs unless otherwise indicated, Student's t-test.

FIG. 7A-C: Antibodies against TGFβ, integrin α3, and integrin αMβ2 rescue the fibrotic damage from UUO insult to mouse kidneys. The descending ureter of the left kidney was surgically ligated, and 50 μg antibody (either tagged with cy7 or unconjugated) was injected i.v. after 7 days. (A) Heart, lung, spleen, kidneys, and liver were harvested 24 hr after injection, and fluorescence intensity was measured via IVIS. (B) Fluorescence of the resected organs was pooled and the percentage of fluorescence associated with healthy and fibrotic kidneys. (C) kidneys were resected 14 post UUO insult (7 days post antibody injection), mounted, and stained via immunohistochemistry for collagen I. The amount of positive IHC staining was compared to the overall amount of kidney tissue, per image. For A and B, n=3. For C, healthy n=10, fibrosis n=10, CBP-antibody treated kidneys n=8, and unconjugated-antibody treated kidneys n=6. *=statistical significance of P<0.05, <0.01, or <0.001, (B) significance is fluorescence of fibrotic left kidney vs fluorescence of healthy kidney, Student's t-test. (C) ANOVA vs fibrosis control, Welch's correction. Comparison between α-αMβ2 and CBP-α-αMβ2 is Student's t-test.

FIG. 8A-D: Antibodies against integrins α3, αM, and αMβ2 de-differentiate myofibroblasts, and remove focal adhesion subcellular structures (FAs). Human monocyte-derived myofibroblasts were treated with 500 ng/ml α-αM, α-αMβ2, α-α3. (A) Untreated population of myofibroblasts, inset on nucleus and focal adhesion at the tip of the myofibroblast. (B) Myofibroblasts treated with α-αM, inset on edge of de-differentiated myofibroblast. Actin staining is diffuse. (C) Myofibroblasts treated with α-αMβ2, inset on the edge of de-differentiated myofibroblast. Actin staining is diffuse. (D) Myofibroblasts treated with α-α3, inset on the edge of de-differentiated myofibroblast. Actin staining is diffuse. Red=talin2, green=αSMA, blue=DAPI. Size bar=30 μm, inset size bar=10 μm.

FIG. 9A-D: Antibodies against integrins α3, αM, and αMβ2 reduce the pro-fibrotic secretome of human myofibroblasts. Human monocyte-derived myofibroblasts and fibroblast-derived myofibroblasts were treated with 500 ng/ml of the indicated antibodies (or 2 ng/ml leukadherin), and the conditioned media after 1 week was assessed via Legendplex ELISA. Secreted proteins over the detection limit were: (A) MCP-1 from monocyte-derived myofibroblasts, (B) MCP-1 from fibroblast-myofibroblasts. (C-D) IL-6 from monocyte-derived myofibroblasts, IL-6 from fibroblast-derived myofibroblasts. n ranges from 3 to 15. *=statistical significance of P<0.05, <0.01, or <0.001, significance vs control, Student's t-test.

FIG. 10A-H: Antibodies against integrins α3, αM, and αMβ2 reduce the pro-fibrotic secretome of mouse myofibroblasts. Mouse monocyte-derived myofibroblasts were treated with 500 ng/ml of α-α3, α-αM, or α-αMβ2, and the conditioned media after 1 week was assessed via Legendplex ELISA. Secreted proteins over the detection limit were: (A) IL-10, (B) IL-23, (C) CCL22, (D) IL-6, (E) CCL17, (F) IL-12 subunit p40, (G) CXCL1, (H) TNF-a. n ranges from 2 to 8. *=statistical significance of P<0.05, <0.01, or <0.001, significance vs control unless otherwise indicated, Student's t-test.

FIG. 11A-D: α-α3 de-differentiates human fibroblast-derived myofibroblasts, while α-αM, and α-αMβ2 do not. Human (A and B) and mouse (C and D) fibroblasts were induced to become myofibroblasts with 5 ng/m TGFb, treated for 1 week with the indicated antibodies at 500 ng/ml, and analyzed via flow cytometry for (A and C) αSMA⁺ and collagen I⁺ double positive cells and (B and D) talin2⁺. n ranges from 2 to 3. *=statistical significance of P<0.05, <0.01, or <0.001, significance vs isotype control antibody, Student's t-test.

FIG. 12A-D: Antibodies against integrins α-α3, α-αM, and α-αMβ2 do not cause cell death. 500 ng/ml of the indicated antibodies (or 2 ng/ml leukadherin) were added to myofibroblasts for 1 week, then cells were assessed for living by live-dead aqua stain. (A) Human monocyte-derived myofibroblasts, (B) human fibroblast-derived myofibroblasts, (C) mouse monocyte-derived myofibroblasts, (D) mouse fibroblast-derived myofibroblasts. *=statistical significance of P<0.05, <0.01, or <0.001, significance vs isotype control antibody, Student's t-test.

FIG. 13A-C: Antibodies against integrins α3, αM, and αMβ2 were conjugated with CBP at approx. 5 peptide copies per antibody molecule. Unlabeled CBP was conjugated to antibodies, and mass change measured via MALDI-TOF: (A) α-α3, (B) α-αM, (C) α-αMβ2. Since the combined molecular weight of the linker and CBP peptide is 2.5 kDa, total numbers of attached CBP peptides were derived.

FIG. 14A-H: Representative images of left, single-lobed lungs stained with Massons's trichrome. (A) Uninsulted lungs instilled with PBS. Lungs insulted with (B-H) 75 g bleomycin, and instilled with (C) α-α3, (D) CBP-α-α3, (E) α-αM, (F) CBP-α-αM, (G) α-αMβ2, (F) CBP-α-αMβ2. Lungs were harvested 3 weeks after insult and stained via Masson's trichrome. Scores in FIGS. 4, 6, and full lungs in FIG. 5.

FIG. 15A-J: Representative images of kidneys, immunohistochemistry stained for collagen I. (A) Uninsulted kidney, (B) untreated UUO kidney after 2 weeks, UUO kidney (2 weeks after UUO, 1 week after antibody injection) treated with 50 micrograms of (C) isotype control antibody, (D) CBP-isotype control antibody, (E) α-TGFβ, (F) CBP-α-TGFβ, (G) α-α3, (H) CBP-α-α3, (I) α-αMβ2, (J) CBP-α-αMβ2. Data in FIG. 7.

FIG. 16A-D: Blood analysis from kidney UUO model. Blood was taken from mice directly before sacrifice at 2 weeks post UUO, 1 week post antibody injection. Concentrations in serum of (A) creatine kinase, (B) creatinine, (C) blood urea nitrogen, and (D) uric acid. *=statistical significance of P<0.05, <0.01, or <0.001, significance vs fibrosis, one way ANOVA, no post test.

FIG. 17A-B: Integrin α3 is upregulated among monocyte-derived cell types found in IPF. (A) Integrin α3 transcript frequency in cells found in IPF. (B) Integrin α3 transcript frequency by individual patient for selected immune cell subtypes. All data and figures from the IPF Atlas, Kraminski/Rosas dataset.

FIG. 18A-B: Integrin αM is upregulated among monocyte-derived cell types found in IPF. (A) Integrin αM transcript frequency in cells found in IPF. (B) Integrin αM transcript frequency by individual patient for selected immune cell subtypes. All data and figures from the IPF Atlas, Kraminski/Rosas dataset.

FIG. 19A-B: Integrin 32 is upregulated among monocyte-derived cell types found in IPF. (A) Integrin 32 transcript frequency in cells found in IPF. (B) Integrin 32 transcript frequency by individual patient for selected immune cell subtypes. All data and figures from the IPF Atlas, Kraminski/Rosas dataset.

FIG. 20: Bleomycin-induced pulmonary fibrosis model

FIG. 21: Mouse weight, insult at day 0, fibrosis treatment times indicated by green arrows.

FIG. 22: Collagen content in the right lung via hydroxyproline assay.

FIG. 23: Fibrosis treatments, Ashcroft score, non-blind scoring.

FIG. 24A-F: Monocyte-to-myofibroblast differentiation required multiple adhesion-related checkpoints to proceed. (A) Freshly isolated human monocytes were cultured with or without pro-fibrotic factors (tryptase, IL-13) for 1 hr, either adhered to a tissue-culture treated surface or in suspension, and then differentiated in fresh medium lacking the pro-fibrotic factors over 3 days into myofibroblasts adherent to the tissue-culture treated surface. Differentiation into myofibroblasts was determined by morphology, namely a highly-elongated, spindle shape. Due to donor variability, readouts for all human monocytes were normalized and compared to the same individual donor's myofibroblast numbers, αSMA and collagen I content. (B) Myofibroblasts were then removed from the surface and assessed for the percentage of αSMA- and collagen I-positive cells by flow cytometry, again normalized for each individual donor. (C) Monocytes plated on fibronectin-coated surfaces differentiated into myofibroblasts, with or without a pro-fibrotic factor, more on stiffer than on softer surfaces. Monocytes cultured at 1 kPa, 12 kPa, and on tissue culture treated plastic (e.g., functionally infinite kPa), each coated with fibronectin, form myofibroblasts at increasing amounts, as measured by (D) morphology (normalized to 12 kPa values, statistics vs 12 kPa) and (E) percentage of cells that are αSMA and collagen I positive by flow cytometry (normalized to 12 kPa values, statistics vs 12 kPa). Due to donor cell variability, readouts were normalized and compared to the number of positive cells at 12 kPa for the individual donor. Donor PBMC, isolated monocytes, and PBMC depleted of monocytes were assessed by flow cytometry and normalized to the 12 kPa readouts as well. Statistics were compared to 12 kPa. (F) Surfaces of 1, 12, and functionally infinite kPa stiffness induced human fibroblast cultures to become increasingly αSMA- and collagen I-positive by flow cytometry. n ranges from 3 to 20. *=statistical significance of P<0.05, <0.01, or <0.001, Statistical comparisons were to the 12 kPa value for each dataset (D-F), 2-way ANOVA for panel C, Sidak post-test, Student's t-test for other panels.

FIG. 25A-B: Increasing surface stiffnesses and pro-fibrotic conditions increased the intensity of talin2 immunostaining in human cells. (A) Freshly purified monocytes, and (B) cultured fibroblasts, were cultured conditions of increasing stiffness and in the presence of pro-fibrotic factors. n ranges from 2 to 8. *=statistical significance of P<0.05, <0.01, or <0.001, Statistical comparisons are to the 12 kPa value for each dataset, Student's t-test.

FIG. 26A-E: Increasing surface stiffnesses increased myofibroblast differentiation and the intensity of talin2 immunostaining in mouse cells. Freshly purified mouse monocytes cultured at 1, 12, and functionally infinite kPa formed myofibroblasts at increasing amounts, as measure by (A) morphology and (B) percentage of cells that are αSMA and collagen I positive. (C) These same populations showed increasing talin2 staining intensity. Mouse fibroblasts cultured under identical conditions also showed increasing numbers of (D) cells that are αSMA and collagen I positive and (E) increased talin2 staining intensity. n ranges from 2 to 8. Statistical comparisons are to the 12 kPa value for each dataset. *=statistical significance of P<0.05, <0.01, or <0.001, Student's t-test.

FIG. 27A-E: Silencing RNA against talin2 dedifferentiated human and mouse myofibroblasts. Freshly purified human monocytes allowed to become myofibroblasts were treated with (A) a mixture of 4 non-targeted siRNAs and talin2 siRNAs at the indicated concentrations. (B) myofibroblasts differentiated from human monocytes were treated with 50 nM mixtures of control siRNA, and siRNA targeting human or mouse talin2, and assessed for (B) the number of αSMA⁺ Collagen I⁺ cells and (C) the amount of talin2. Myofibroblasts differentiated from mouse monocytes were treated identically, except that individual siRNA was also used in addition to the mixtures, and were assessed for (D) the number of αSMA⁺ Collagen I⁺ cells and (E) the amount of talin2. n ranges from 2 to 10. *=statistical significance of P<0.05, <0.01, or <0.001, 2-way ANOVA for panel A, Sidak post-test, Student's t-test for other panels.

FIG. 28A-D: Silencing RNA against talin2 dedifferentiated human and mouse myofibroblasts. Fibroblasts were cultured on an infinite kPa tissue culture surface and treated with TGF-β, inducing them to become myofibroblasts. These myofibroblasts were treated with 50 nM mixtures of 4 control siRNAs, mixtures of 4 siRNAs targeting human talin2, mixtures of 4 siRNAs targeting mouse talin2, or 4 individual siRNAs targeting mouse talin2. Human myofibroblasts were assessed for (A) the number of αSMA⁺ Collagen I⁺ cells and (B) the amount of talin2, and mouse myofibroblasts were assessed (C) the number of αSMA⁺ Collagen I⁺ cells and (D) the amount of talin2. n ranges from 2 to 5. *=statistical significance of P<0.05, <0.01, or <0.001, Student's t-test.

FIG. 29A-L: Talin2 siRNA rescued the fibrotic damage from bleomycin insult to mouse lungs. 50 μl of 0.2 μM talin2 siRNA was administered to mouse lungs 7, 9, 11, 14, 16, and 18 days after insult by bleomycin. (A) Mouse weights after treatment. (B) Collagen content from the right, multi-lobed lung assessed by hydroxyproline assay. (C) Data from C divided by dry weight of right lobes of mouse lungs. (D) Blinded Ashcroft scoring. (E-H) Representative images of left, single lobed lungs stained with Massons's trichrome. (I-L) Inset of lungs. (E, I) Uninjured lung, (F, J) fibrotic lung, (G, K) fibrotic lung treated with siRNA control, and (H, L) fibrotic lung treated with talin2 siRNA. n ranges from 6 to 8. *=statistical significance of P<0.05, <0.01, or <0.001, significance vs fibrotic lungs unless otherwise indicated, 2-way ANOVA for panel A, Sidak post-test, Student's t-test for other panels.

FIG. 30A-E: Tln2−/− mice were resistant to UUO-induced kidney fibrosis. Mouse left kidneys were injured by UUO ligation. Kidneys were sectioned and IHC stained using α-collagen-I as a fibrosis marker. (A) Right, healthy C57B16 kidney. (B) Left, fibrotic C57B16 kidney. (C) Right, healthy Tln2−/− kidney. (D) Left, fibrotic Tln2−/− kidney. (E) The ratio of fibrotic to healthy tissue in each lung section. n=6. *=statistical significance of P<0.05, <0.01, or <0.001, Student's t-test.

FIG. 31: The addition of siRNA-AF-488 to human monocyte-myofibroblasts showed an increase in the amount of green fluorescence in cells.

FIG. 32A-D: Treatment with talin2 siRNA altered the morphology of myofibroblasts, as well as the localization of talin2. Human monocyte-derived myofibroblasts were treated with 50 nM siRNA targeting talin2. (A) A myofibroblast representative of morphology. (B) Untreated population of myofibroblasts, inset on nucleus and focal adhesion at the tip of the myofibroblast. (C) Untreated population of myofibroblasts, clear talin2 localization at the periphery of the cell. (D) Control siRNA treated myofibroblasts, inset on tip of myofibroblast. Talin2 siRNA treated myofibroblasts, inset on edges of de-differentiated myofibroblasts. Red=talin2, green=αSMA, blue=DAPI. Size bar=30 μm, inset size bar=10 μm.

FIG. 33A-J: Treatment with talin2 siRNA reduced the pro-fibrotic secretome of mouse myofibroblasts. Treatment with 50 nM of talin2 siRNA on mouse monocyte-derived myofibroblasts did not affect the amount of secreted (A) IL-10, but reduced the amount of secreted (B) IL-23, (C) CCL22, (D) IL-6, (E) CCL17, (F) IL-12 subunit p40, (G) CXCL1, (I) TNF-α, and (J) IL-10. (H) was from the secretome of human fibroblast-myofibroblasts. n ranges from 3 to 4. *=statistical significance of P<0.05, <0.01, or <0.001, significance vs control unless otherwise indicated, Student's t-test.

FIG. 34A-D: Treatment with talin2 siRNA reduced the pro-fibrotic secretome of human myofibroblasts. Treatment with 50 nM of talin2 siRNA on human monocyte-myofibroblasts significantly lowered the amount of secreted (A) MCP-1 and lowered, though not significantly, the amount of (B) TNF-α. Talin2 siRNA reduced the amount of IL-6 secreted by (C) monocyte-myofibroblasts and (D) fibroblast-myofibroblasts. *=statistical significance of P<0.05, <0.01, or <0.001, significance vs control unless otherwise indicated, Student's t-test.

FIG. 35A-F: talin2 siRNA rescued the fibrotic damage from both male and female mice. Talin2 siRNA treatment compared across male (n=5 or 6, A, B, C) and female (n=3, D, E, F) mice for amount of collagen in lungs (A, D), collagen as a percentage of dry lung weight (B, E), and blinded Ashcroft scores (C, F). n ranges from 3 to 6. *=statistical significance of P<0.05, <0.01, or <0.001, significance vs fibrotic lungs unless otherwise indicated, Student's t-test.

FIG. 36A-F: Treatment with talin2 siRNA showed a trend to reduction of the number of CD45+αSMA+ and CD45+ talin2+ cells compared with fibrotic lung. Mouse lungs were insulted with bleomycin, and treated with 0.2 μM of talin2 siRNA. Lungs were lavaged after euthanasia, and assessed for (A) CD45, (B) talin2, and (C) αSMA by flow cytometry. (D) CD45⁺ αSMA⁺ cells normalized to overall CD45+ cells from that mouse. (E) CD45⁺ Talin2⁺ cells normalized to overall CD45⁺ cells from that mouse. (F) Representative flow plot of BAL cells. *=statistical significance of P<0.05, <0.01, or <0.001, significance vs control unless otherwise indicated, Student's t-test.

FIG. 37A-G: Tln2−/− mice are resistant to lung fibrosis. Tln2−/− mice were insulted by bleomycin, and assessed as in FIG. 28. (A) Collagen content from the right, multi-lobed lung assessed by hydroxyproline assay. (B) Data from C divided by dry weight of right lobes of mouse lungs. (C) Blinded Ashcroft scoring. (D-E) Representative images of left, single lobed lungs stained with Massons's trichrome. (F-G) Inset of lungs. (D,F) Uninjured Tln2−/− lung, (E,G) bleomycin-insulted Tln2−/− lung. n=6. *=statistical significance of P<0.05, <0.01, or <0.001, significance vs fibrotic lungs unless otherwise indicated, Student's t-test.

FIG. 38A-F: Blood analysis from kidney UUO model. Blood was taken from mice (C57B16 and Tln2−/−) directly before sacrifice at 2 weeks post UUO. Concentrations in serum of (A) creatine kinase, (B) creatinine, (C) blood urea nitrogen, (D) bilirubin, (E) ALT, and (F) AST. *=statistical significance of P<0.05, <0.01, or <0.001, Student's t-test.

FIG. 39A-B: Talin2 transcripts were upregulated among monocyte-derived cell types found in IPF. (A) Talin2 transcript frequency in cells found in IPF. (B) Talin2 transcript frequency by individual patient for selected immune cell subtypes. All data and figures from the IPF Atlas, Kraminski/Rosas dataset.

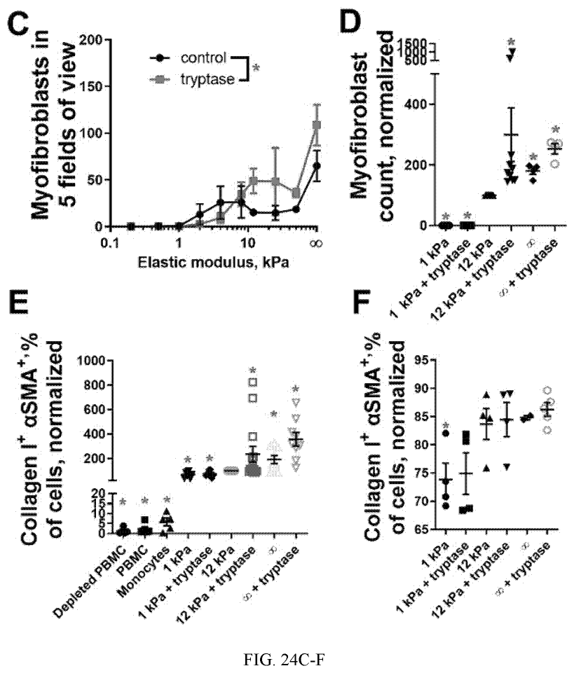

FIG. 24A-.

### DETAILED DESCRIPTION OF THE INVENTION

The inventors have developed a novel anti-fibrotic (silencing RNA against Talin2) capable of reversing myofibroblast differentiation in vitro and fibrosis in vivo. Talin2 is a protein that is involved in the adhesion mechanics of cells. Specifically, talin2 senses the stiffness of surfaces. This novel anti-fibrotics were developed from a study of how myofibroblast differentiation is governed by properties of cell adhesion. It was found that monocytes and fibroblasts cultured on soft surfaces cannot be differentiated into myofibroblasts, which are cells key to scar tissue formation in fibrosis. The inventors assessed the RNA content of monocytes cultured on soft and stiff surfaces. They identified several proteins upregulated in cells cultured on stiff compared to soft surfaces: including integrin αM, Integrin αMβ2, Integrin α3, and talin2. Integrins are key in the development of focal adhesions that bind cells to surfaces. Integrins are composed of alpha (α) and beta (β) subunits, which combine to form heterodimers that recognize specific soluble factors and ECM proteins. Antibodies that block αM, αMβ2, α3 reverse existing fibrosis. Attachment of decorin's collagen-binding peptide (CBP) targets these antibodies to collagen-rich scar tissue, and the inventors can increase the local concentration of anti-integrin antibodies with CBP by twofold in fibrotic organs. They further tested a monoclonal antibody that recognizes only the activation epitope of αMβ2, clone CBRM1/5. This antibody binds only to a conformational epitope exposed on activated αMβ2. By using an antibody that recognizes only a conformational epitope, the inventors hope to further reduce the side-effects of anti-integrin treatments. To neutralize Talin2, the inventors used silencing RNA against Talin2 (Talin2 siRNA) to neutralize Talin2. Talin2 siRNA also reverses existing fibrosis in fibrotic mouse organs. siRNA is easily delivered in powdered, inhaled form to the lungs.

### I. ANTIBODIES

In certain aspects, an antibody or a fragment thereof that binds to at least a portion of an integrin protein or integrin complex and inhibits or blocks the binding of the integrin receptor with its ligand.

In some aspects, the antibody is a monoclonal antibody or a polyclonal antibody. In some aspects, the antibody is a chimeric antibody, an affinity matured antibody, a humanized antibody, or a human antibody. In some aspects, the antibody is an antibody fragment. In some aspects, the antibody is a Fab, Fab′, Fab′-SH, F(ab′)2, or scFv. In one aspect, the antibody is a chimeric antibody, for example, an antibody comprising antigen binding sequences from a non-human donor grafted to a heterologous non-human, human or humanized sequence (e.g., framework and/or constant domain sequences). In one aspect, the non-human donor is a mouse. In one aspect, an antigen binding sequence is synthetic, e.g., obtained by mutagenesis (e.g., phage display screening, etc.). In one aspect, a chimeric antibody has murine V regions and human C region. In one aspect, the murine light chain V region is fused to a human kappa light chain or a human IgG1 C region.

Examples of antibody fragments include, without limitation: (i) the Fab fragment, consisting of VL, VH, CL and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multispecific fragments constructed by gene fusion (U.S. Patent Pub. 2005/0214860). Fv, scFv or diabody molecules may be stabilized by the incorporation of disulphide bridges linking the VH and VL domains. Minibodies comprising a scFv joined to a CH3 domain may also be made (Hu et al, 1996).

A monoclonal antibody is a single species of antibody wherein every antibody molecule recognizes the same epitope because all antibody producing cells are derived from a single B-lymphocyte cell line. Hybridoma technology involves the fusion of a single B lymphocyte from a mouse previously immunized with an antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced. However, in therapeutic applications a goal of hybridoma technology is to reduce the immune reaction in humans that may result from administration of monoclonal antibodies generated by the non-human (e.g. mouse) hybridoma cell line.

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

By known means as described herein, polyclonal or monoclonal antibodies, binding fragments and binding domains and CDRs (including engineered forms of any of the foregoing), may be created that are specific to an integrin protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. Particularly, the antibodies may be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by this reference. These techniques are further described in: Marks (1992); Stemmer (1994); Gram et al. (1992); Barbas et al. (1994); and Schier et al. (1996).

Methods for producing polyclonal antibodies in various animal species, as well as for producing monoclonal antibodies of various types, including humanized, chimeric, and fully human, are well known in the art. Methods for producing these antibodies are also well known. For example, the following U.S. patents and patent publications provide enabling descriptions of such methods and are herein incorporated by reference: U.S. Patent publication Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent publications, and other publications cited herein and therein are hereby incorporated by reference in the present application.

It is fully expected that antibodies will have the ability to neutralize, block, or counteract the effects of the integrins or integrin complexes, regardless of the animal species, monoclonal cell line or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into binding fragments having the binding domain or CDR. Removal of the Fc portion reduces the likelihood that the antigen binding fragment will elicit an undesirable immunological response and, thus, antibodies without Fc may be particularly useful for prophylactic or therapeutic treatments. As described above, antibodies may also be constructed so as to be chimeric, partially or fully human, so as to reduce or eliminate the adverse immunological consequences resulting from administering to an animal an antibody that has been produced in, or has sequences from, other species.

### A. Complementarity Determining Regions of Antibodies

The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope. 2) Hydrogen-deuterium exchange and mass spectroscopy. 3) Polypeptide fragmentation and peptide mapping approaches in which one generates multiple overlapping peptide fragments from the full length of the polypeptide and evaluates the binding affinity of these peptides for the epitope. 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

### B. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain aspects, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other aspects, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the present disclosure to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### C. Conjugation of Antibodies

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

### 1. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

### 2. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### D. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various aspects can also be synthesized in solution or on a solid support in accordance with conventional techniques. See, for example, Stewart and Young, (1984); Tam et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some aspects, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with aspects include, but are not limited to, IL-1, IL-2, IL-4, IL-7, IL-12, γ-interferon, GMCSF, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m²) (Johnson/Mead, NJ), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### E. Fully Human Antibody Production

Methods are available for making fully human antibodies. Using fully human antibodies can minimize the immunogenic and allergic responses that may be caused by administering non-human monoclonal antibodies to humans as therapeutic agents. In one aspect, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of humanized antibodies include, but are not limited to, detect a cell expressing an anticipated protein, either in vivo or in vitro, pharmaceutical preparations containing the antibodies of the present disclosure, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MIPL- and msk-receptors using such an approach.

### F. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH2NH—, —CH₂S—, —CH2-CH2-, —CH═CH— (cis and trans), COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain aspects of the disclosure to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used

### II. ECM AFFINITY PEPTIDES

Aspects of the disclosure relate to ECM-affinity peptides. The table below describes ECM-affinity peptides useful in the methods and compositions of the disclosure:

| | | SEQ ID |
|---|---|---|
| Description | Sequence | NO: |
| Residues 17- | GPFQQRGLFDFMLEDEASGIGPEVPDDRDFEPSLGPVCPFR | 1 |
| 359 of decorin | CQCHLRVVQCSDLGLDKVPKDLPPDTTLLDLQNNKITEIKD | |
| | GDFKNLKNLHALILVNNKISKVSPGAFTPLVKLERLYLSKN | |
| | QLKELPEKMPKTLQELRAHENEITKVRKVTFNGLNQMIVIE | |
| | LGTNPLKSSGIENGAFQGMKKLSYIRIADTNITSIPQGLPPSL | |
| | TELHLDGNKISRVDAASLKGLNNLAKLGLSFNSISAVINGS | |
| | LANTPHLRELHLDNNKLTRVPGGLAEHKYIQVVYLHNNNI | |
| | SVVGSSDFCPPGHNTKKASYSGVSLFSNPVQYWEIQPSTFR | |
| | CVYVRSAIQLGNYK | |
| Decorin | LRELHLNNNC | 2 |
| peptide from | | |
| bovine | | |
| Decorin | LRELHLDNNC | 3 |
| peptide from | | |
| human | | |
| Human vWF | MIPARFAGVLLALALILPGTLCAEGTRGRSSTARCSLFGSDF | 4 |
| | VNTFDGSMYSFAGYCSYLLAGGCQKRSFSIIGDFQNGKRVS | |
| | LSVYLGEFFDIHLFVNGTVTQGDQRVSMPYASKGLYLETE | |
| | AGYYKLSGEAYGFVARIDGSGNFQVLLSDRYFNKTCGLCG | |
| | NFNIFAEDDFMTQEGTLTSDPYDFANSWALSSGEQWCERA | |
| | SPPSSSCNISSGEMQKGLWEQCQLLKSTSVFARCHPLVDPE | |
| | PFVALCEKTLCECAGGLECACPALLEYARTCAQEGMVLYG | |
| | WTDHSACSPVCPAGMEYRQCVSPCARTCQSLHINEMCQER | |
| | CVDGCSCPEGQLLDEGLCVESTECPCVHSGKRYPPGTSLSR | |
| | DCNTCICRNSQWICSNEECPGECLVTGQSHFKSFDNRYFTF | |
| | SGICQYLLARDCQDHSFSIVIETVQCADDRDAVCTRSVTVR | |
| | LPGLHNSLVKLKHGAGVAMDGQDVQLPLLKGDLRIQHTV | |
| | TASVRLSYGEDLQMDWDGRGRLLVKLSPVYAGKTCGLCG | |
| | NYNGNQGDDFLTPSGLAEPRVEDFGNAWKLHGDCQDLQK | |
| | QHSDPCALNPRMTRFSEEACAVLTSPTFEACHRAVSPLPYL | |
| | RNCRYDVCSCSDGRECLCGALASYAAACAGRGVRVAWRE | |
| | PGRCELNCPKGQVYLQCGTPCNLTCRSLSYPDEECNEACLE | |
| | GCFCPPGLYMDERGDCVPKAQCPCYYDGEIFQPEDIFSDHH | |
| | TMCYCEDGFMHCTMSGVPGSLLPDAVLSSPLSHRSKRSLS | |
| | CRPPMVKLVCPADNLRAEGLECTKTCQNYDLECMSMGCV | |
| | SGCLCPPGMVRHENRCVALERCPCFHQGKEYAPGETVKIG | |
| | CNTCVCRDRKWNCTDHVCDATCSTIGMAHYLTFDGLKYL | |
| | FPGECQYVLVQDYCGSNPGTFRILVGNKGCSHPSVKCKKR | |
| | VTILVEGGEIELFDGEVNVKRPMKDETHFEVVESGRYIILLL | |
| | GKALSVVWDRHLSISVVLKQTYQEKVCGLCGNFDGIQNND | |
| | LTSSNLQVEEDPVDFGNSWKVSSQCADTRKVPLDSSPATC | |
| | HNNIMKQTMVDSSCRILTSDVFQDCNKLVDPEPYLDVCIY | |
| | DTCSCESIGDCACFCDTIAAYAHVCAQHGKVVTWRTATLC | |
| | PQSCEERNLRENGYECEWRYNSCAPACQVTCQHPEPLACP | |
| | VQCVEGCHAHCPPGKILDELLQTCVDPEDCPVCEVAGRRF | |
| | ASGKKVTLNPSDPEHCQICHCDVVNLTCEACQEPGGLVVP | |
| | PTDAPVSPTTLYVEDISEPPLHDFYCSRLLDLVFLLDGSSRL | |
| | SEAEFEVLKAFVVDMMERLRISQKWVRVAVVEYHDGSHA | |
| | YIGLKDRKRPSELRRIASQVKYAGSQVASTSEVLKYTLFQIF | |
| | SKIDRPEASRITLLLMASQEPQRMSRNFVRYVQGLKKKKVI | |
| | VIPVGIGPHANLKQIRLIEKQAPENKAFVLSSVDELEQQRDE | |
| | IVSYLCDLAPEAPPPTLPPDMAQVTVGPGLLGVSTLGPKRN | |
| | SMVLDVAFVLEGSDKIGEADFNRSKEFMEEVIQRMDVGQD | |
| | SIHVTVLQYSYMVTVEYPFSEAQSKGDILQRVREIRYQGGN | |
| | RTNTGLALRYLSDHSFLVSQGDREQAPNLVYMVTGNPASD | |
| | EIKRLPGDIQVVPIGVGPNANVQELERIGWPNAPILIQDFETL | |
| | PREAPDLVLQRCCSGEGLQIPTLSPAPDCSQPLDVILLLDGS | |
| | SSFPASYFDEMKSFAKAFISKANIGPRLTQVSVLQYGSITTID | |
| | VPWNVVPEKAHLLSLVDVMQREGGPSQIGDALGFAVRYL | |
| | TSEMHGARPGASKAVVILVTDVSVDSVDAAADAARSNRV | |
| | TVFPIGIGDRYDAAQLRILAGPAGDSNVVKLQRIEDLPTMV | |
| | TLGNSFLHKLCSGFVRICMDEDGNEKRPGDVWTLPDQCHT | |
| | VTCQPDGQTLLKSHRVNCDRGLRPSCPNSQSPVKVEETCG | |
| | CRWTCPCVCTGSSTRHIVTFDGQNFKLTGSCSYVLFQNKEQ | |
| | DLEVILHNGACSPGARQGCMKSIEVKHSALSVELHSDMEV | |
| | TVNGRLVSVPYVGGNMEVNVYGAIMHEVRFNHLGHIFTFT | |
| | PQNNEFQLQLSPKTFASKTYGLCGICDENGANDFMLRDGT | |
| | VTTDWKTLVQEWTVQRPGQTCQPILEEQCLVPDSSHCQVL | |
| | LLPLFAECHKVLAPATFYAICQQDSCHQEQVCEVIASYAHL | |
| | CRTNGVCVDWRTPDFCAMSCPPSLVYNHCEHGCPRHCDG | |
| | NVSSCGDHPSEGCFCPPDKVMLEGSCVPEEACTQCIGEDGV | |
| | QHQFLEAWVPDHQPCQICTCLSGRKVNCTTQPCPTAKAPT | |
| | CGLCEVARLRQNADQCCPEYECVCDPVSCDLPPVPHCERG | |
| | LQPTLTNPGECRPNFTCACRKEECKRVSPPSCPPHRLPTLRK | |
| | TQCCDEYECACNCVNSTVSCPLGYLASTATNDCGCTTTTC | |
| | LPDKVCVHRSTIYPVGQFWEEGCDVCTCTDMEDAVMGLR | |
| | VAQCSQKPCEDSCRSGFTYVLHEGECCGRCLPSACEVVTG | |
| | SPRGDSQSSWKSVGSQWASPENPCLINECVRVKEEVFIQQR | |
| | NVSCPQLEVPVCPSGFQLSCKTSACCPSCRCERMEACMLN | |
| | GTVIGPGKTVMIDVCTTCRCMVQVGVISGFKLECRKTTCNP | |
| | CPLGYKEENNTGECCGRCLPTACTIQLRGGQIMTLKRDETL | |
| | QDGCDTHFCKVNERGEYFWEKRVTGCPPFDEHKCLAEGG | |
| | KIMKIPGTCCDTCEEPECNDITARLQYVKVGSCKSEVEVDI | |
| | HYCQGKCASKAMYSIDINDVQDQCSCCSPTRTEPMQVALH | |
| | CTNGSVVYHEVLNAMECKCSPRKCSK | |
| vWF A3 | CSGEGLQIPTLSPAPDCSQPLDVILLLDGSSSFPASYFDEMKS | 5 |
| domain; 1670- | FAKAFISKANIGPRLTQVSVLQYGSITTIDVPWNVVPEKAHL | |
| 1874 of vWF | LSLVDVMQREGGPSQIGDALGFAVRYLTSEMHGARPGASK | |
| (907-1111 of | AVVILVTDVSVDSVDAAADAARSNRVTVFPIGIGDRYDAA | |
| mature VWF) | QLRILAGPAGDSNVVKLQRIEDLPTMVTLGNSFLHKLCSG | |
| vWF A3: | CSQPLDVILLLDGSSSFPASYFDEMKSFAKAFISKANIGPRLT | 6 |
| 1686-1881 of | QVSVLQYGSITTIDVPWNVVPEKAHLLSLVDVMQREGGPS | |
| VWF (923- | QIGDALGFAVRYLTSEMHGARPGASKAVVIL VTDVSVDSV | |
| 1118 of mature | DAAADAARSNRVTVFPIGIGDRYDAAQLRILAGPAGDSNV | |
| VWF) | VKLQRIEDLPTMVTLGNSFLHKLCSGFVRICTG | |
| vWF A1 | CQEPGGLVVPPTDAPVSPTTLYVEDISEPPLHDFYCSRLLDL | 7 |
| domain: 1237- | VFLLDGSSRLSEAEFEVLKAFVVDMMERLRISQKWVRVAV | |
| 1458 VWF | VEYHDGSHAYIGLKDRKRPSELRRIASQVKYAGSQVASTSE | |
| (474-695 of | VLKYTLFQIFSKIDRPEASRITLLLMASQEPQRMSRNFVRYV | |
| mature VWF) | QGLKKKKVIVIPVGIGPHANLKQIRLIEKQAPENKAFVLSSV | |
| | DELEQQRDEIVSYLC | |
| PIGF2 | MPVMRLFPCFLQLLAGLALPAVPPQQWALSAGNGSSEVEV | 8 |
| | VPFQEVWGRSYCRALERLVDVVSEYPSEVEHMFSPSCVSL | |
| | LRCTGCCGDENLHCVPVETANVTMQLLKIRSGDRPSYVEL | |
| | TFSQHVRCECRPLREKMKPERRRPKGRGKRRREKQRPTDC | |
| | HLCGDAVPRR | |
| PIGF2 peptide | RRRPKGRGKRRREKQRPTDCHLCGDAVPRR | 9 |
| PIGF2 peptide | RRRPKGRGKRRREKQRPTDCHL | 10 |
| PIGF2 peptide | RRPKGRGKRRREKQRPTD | 11 |
| PIGF2 peptide | RRRPKGRGKRRREKQ | 12 |
| PIGF2 peptide | GKRRREKQ | 13 |
| PIGF2 peptide | RRRPKGRG | 14 |
| PIGF2 peptide | RRKTKGKRKRSRNSQTEEPHP | 15 |
| CXCL-12γ | KPVSLSYRCPCRFFESHVARANVKHLKILNTPNCALQIVAR | 16 |
| | LKNNNRQVCIDPKLKWIQEYLEKALNKGRREEKVGKKEKI | |
| | GKKKRQKKRKAAQKRKN | |
| CXCL-12γ | GRREEKVGKKEKIGKKKRQKKRKAAQKRKN | 17 |
| peptide | | |

The ECM-affinity peptide may be a peptide with 75, 76, 77, 78, 79, 80, 81, 82, 83, 84, 85, 86, 87, 88, 89, 90, 91, 92, 93, 94, 95, 96, 97, 98, 99, or 10000 identity (or any derivable range therein) to a peptide of the disclosure. The peptide or polypeptide may have one or more conservative or non-conservative substitutions. Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

The polypeptides described herein may include 1, 2, 3, 4, 5, 6, 7, 8, 9, 10, 11, 12, 13, 14, 15, or more (or any derivable range therein) variant amino acids within at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of a peptide or polypeptide of the disclosure.

A polypeptide segment as described herein may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein of a peptide or polypeptide of the disclosure.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

A linker sequence may be included in the antibody-peptide construction. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate that antibody and the peptide.

The ECM-affinity peptides of the disclosure may have affinity to one or more components of the extracellular matrix such as fibronectin, collagen, (collagen type I, collagen type III, and/or collagen type IV), tenascin C, fibrinogen, and fibrin.

### III. PEPTIDE, POLYPEPTIDES, AND PROTEINS

As used herein, a “protein” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS:1-37.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391,392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS:1-37.

In some aspects, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-37.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804,805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-37 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-37.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332,333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807,808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-37 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-37.

In some aspects there is a nucleic acid or polypeptide region starting and ending at any of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329,330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 within any of SEQ ID NOS:1-37, and the nucleic acid or polypeptide region is or is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to the same region in SEQ ID NOS:1-37.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within 2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some aspects of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### IV. NUCLEIC ACID AND OLIGONUCLEOTIDE ASPECTS

The term “nucleoside” refers to a unit made up of a heterocyclic base and its sugar. The term “nucleotide” refers to a nucleoside having a phosphate group on its 3′ or 5′ sugar hydroxyl group. The term “oligonucleotide” or “nucleic acid” refers to a plurality of joined nucleotide units formed in a specific sequence from naturally occurring bases and pentofuranosyl groups joined through a sugar group by native phosphodiester bonds. This term refers to both naturally occurring and synthetic species formed from naturally occurring subunits.

### A. Inhibitory Nucleic Acids

The Table below provides exemplary inhibitory nucleic acids to Talin2:

| Description | Sequence | SEQ ID NO: |
|---|---|---|
| TALIN2 siRNA#1 | UAAUCGUUCAGUUUUCUGG | 18 |
| TALIN2 siRNA#2 | AAAUCUUUAAGGACAGGGC | 19 |
| TALIN2 siRNA#3 | GAGUAUUUAACCACAGUCG | 20 |
| TALIN2 siRNA#4 | AAACUCACAAGCUUUCUCG | 21 |
| TALIN2 siRNA#1-DNA | TAATCGTTCAGTTTTCTGG | 22 |
| TALIN2 siRNA#2-DNA | AAATCTTTAAGGACAGGGC | 23 |
| TALIN2 siRNA#3-DNA | GAGTATTTAACCACAGTCG | 24 |
| TALIN2 siRNA#4-DNA | AAACTCACAAGCTTTCTCG | 25 |
| TALIN2 Target #1 | CCAGAAAACUGAACGAUUA | 29 |
| TALIN2 Target #2 | GCCCUGUCCUUAAAGAUUU | 30 |
| TALIN2 Target #3 | CGACUGUGGUUAAAUACUC | 31 |
| TALIN2 Target #4 | CGAGAAAGCUUGUGAGUUU | 32 |
| TALIN2 Target #1-DNA | CCAGAAAACTGAACGATTA | 33 |
| TALIN2 Target #2-DNA | GCCCTGTCCTTAAAGATTT | 34 |
| TALIN2 Target #3-DNA | CGACTGTGGTTAAATACTC | 35 |
| TALIN2 Target #4-DNA | CGAGAAAGCTTGTGAGTTT | 36 |

In some aspects, the disclosure relates to inhibitory nucleic acids/oligonucleotides that inhibit the gene expression of Talin2. Examples of an inhibitory oligonucleotides include but are not limited to siRNA (small interfering RNA), short hairpin RNA (shRNA), double-stranded RNA, an antisense oligonucleotide, a ribozyme, and an oligonucleotide encoding any thereof. An inhibitory oligonucleotide may inhibit the transcription of a gene or prevent the translation of a gene transcript in a cell. An inhibitory oligonucleotide acid may be from 16 to 1000 nucleotides long, and in certain aspects from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of Talin2 by at least 10%, 20%, 30%, or 40%, more particularly by at least 50%, 60%, or 70%, and most particularly by at least 75%, 80%, 90%, 95%, 99%, or 100% more or any range or value in between the foregoing.

In further aspects, there are synthetic oligonucleotides that are Talin2 inhibitors. An inhibitor may be between 17 to 25 nucleotides in length and comprises a 5′ to 3′ sequence that is at least 90% complementary to the 5′ to 3′ sequence of a mature Talin2 mRNA. In certain aspects, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature Talin2 mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some aspects, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylamines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some aspects, the oligonucleotide may be further defined as having one or more locked nucleotides, ethylene bridged nucleotides, peptide nucleic acids, or a 5′(E)-vinyl-phosphonate (VP) modification. In some aspects, the oligonucleotides has one or more phosphorothioated DNA or RNA bases.

### B. Nucleic Acids Aspects

In certain aspects the size of a nucleic acid may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 nucleic acid residues or greater, and any range derivable therein.

The nucleic acids of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more modified nucleic acids. In some aspects, the nucleic acid of the disclosure may be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical in sequence with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 contiguous nucleic acids, or any range derivable therein, of SEQ ID NOS:18-25, 29-36 or nucleic acids encoding the polypeptides and peptides of SEQ ID NOS:1-17.

In some aspects, the nucleic acid may comprise nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) of SEQ ID NOS:18-25, 29-36 or nucleic acids encoding the polypeptides and peptides of SEQ ID NOS: 1-17.

In some aspects, the nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) contiguous nucleic acids of SEQ ID NOS:18-25, 29-36 or nucleic acids encoding the polypeptides and peptides of SEQ ID NOS:1-17.

In some aspects, the nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) contiguous nucleic acids of SEQ ID NOS:18-25, 29-36 or nucleic acids encoding the polypeptides and peptides of SEQ ID NOS:1-17 that comprise at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity with one of SEQ ID NOS:18-25, 29-36 or nucleic acids encoding the polypeptides and peptides of SEQ ID NOS:1-17.

In some aspects there is a nucleic acid molecule starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138,139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 of any of SEQ ID NOS:18-25, 29-36 or nucleic acids encoding the polypeptides and peptides of SEQ ID NOS:1-17 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318,319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) contiguous nucleotides of any of SEQ ID NOS:18-25, 29-36 or nucleic acids encoding the polypeptides and peptides of SEQ ID NOS:1-17.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” or “nucleic acid” are used interchangeable and refer to a nucleic acid molecule that may be recombinant or synthetically synthesized. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that may encode a protein, polypeptide, or peptide, or a region thereof, or a complement to a protein, peptide or region of a protein, such as a region of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous amino acids of a region or complement of a region of a gene or mRNA (either coding or non-coding region).

As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length, may be combined with other nucleic acid sequences, such as promoters, polyadenylation signals, additional restriction enzyme sites, multiple cloning sites, coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### C. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### D. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred aspect, the nucleic acid molecules are oligonucleotides. In a more preferred aspect, the oligonucleotides are from highly variable regions of the heavy and light chains of the antibody of interest. In an even more preferred aspect, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### V. OBTAINING ENCODED POLYPEPTIDE ASPECTS

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### VI. ADMINISTRATION OF THERAPEUTIC COMPOSITIONS

The therapy provided herein may comprise administration of a combination of therapeutic agents, such as a first therapy and a second therapy. The therapies may be administered in any suitable manner known in the art. For example, the first and second treatment may be administered sequentially (at different times) or concurrently (at the same time). In some aspects, the first and second treatments are administered in a separate composition. In some aspects, the first and second treatments are in the same composition.

In some aspects, the first therapy and the second therapy are administered substantially simultaneously. In some aspects, the first therapy and the second therapy are administered sequentially. In some aspects, the first therapy, the second therapy, and a third therapy are administered sequentially. In some aspects, the first therapy is administered before administering the second therapy. In some aspects, the first therapy is administered after administering the second therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, the first therapy comprises a first protein, a nucleic acid encoding for the first protein, a vector comprising the nucleic acid encoding for the first protein, or a cell comprising the first protein, a nucleic acid encoding for the first protein, or a vector comprising the nucleic acid encoding for the first protein. In some aspects, a single dose of the first protein therapy is administered. In some aspects, multiple doses of the first protein are administered. In some aspects, the first protein is administered at a dose of between 1 mg/kg and 5000 mg/kg. In some aspects, the first protein is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121,122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some aspects, a single dose of the second therapy is administered. In some aspects, multiple doses of the second therapy are administered. In some aspects, the second therapy is administered at a dose of between 1 mg/kg and 100 mg/kg. In some aspects, the second therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. Aspects include methods of administering an effective amount or dose or compositions formulated with an effective dose. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In some aspects, pharmaceutical compositions are administered to a subject. Different aspects may involve administering an effective amount of a composition to a subject. In some aspects, an antibody or antigen binding fragment capable of binding to [protein of interest] may be administered to the subject to protect against or treat a condition (e.g., fibrosis). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

Upon formulation, solutions will be administered in a manner compatible with the dosage formulation and in such amount as is therapeutically or prophylactically effective. The formulations are easily administered in a variety of dosage forms, such as the type of injectable solutions described above.

### VII. EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: Blocking Antibodies Against Integrin-α3, -αM, and -αMβ2 De-Differentiate Myofibroblasts and Reverse Lung and Kidney Fibroses

Fibrosis is involved in 45% of deaths in the United States, and no treatment exists to reverse the progression of the disease. Myofibroblasts are key to the progression and maintenance of fibrosis. The inventors investigated features of cell adhesion necessary for monocytes to differentiate into myofibroblasts, seeking to identify pathways key to myofibroblast differentiation. Blocking antibodies against integrins α3, αM, and αMβ2 de-differentiate myofibroblasts in vitro, lower the pro-fibrotic secretome of myofibroblasts, and reverse lung and kidney fibrosis in vivo. Decorin's collagen-binding peptide directs blocking antibodies (against integrins-α3, -αM, -αMβ2) to both fibrotic lungs and fibrotic kidneys, reducing the dose of antibody necessary to reverse fibrosis. This targeted immunotherapy blocking key integrins may be an effective therapeutic for the treatment and reversal of fibrosis.

### A. Introduction

Fibrosis is defined by dysregulated extracellular matrix (ECM) deposition leading to scar tissue deposition and increases in tissue stiffness [1]. Fibrosing diseases—including pulmonary fibrosis, congestive heart failure, liver cirrhosis, and end-stage kidney disease—are involved in 45% of deaths in the United States [1, 2]. There are only 2 FDA approved treatments for fibrosis, pirfenidone and nintedanib [2, 3]. Pirfenidone and nintedanib slow, but do not reverse, the progression of fibrosis [4], with mechanisms of action that are poorly understood [5].

A major goal of research in fibrosis is developing a treatment capable of reversing established fibrosis [1]. To the inventors' knowledge, only one treatment (recombinant pentraxin-2, PRM-151) has thus far shown even a modest ability to reverse even some symptoms of fibrosis in some patients [6]. To fully reverse fibrosis, several cell and ECM-related changes must occur: myofibroblasts must de-activate to reduce the stiffness of the tissue, collagen deposition must cease, and existing ECM must be remodeled. Interrupting collagen deposition alone can destabilize scar tissue [1] and monocyte-derived cells are capable of removing deposited ECM while regenerating tissue [7].

Here the inventors show that CBP-functionalized blocking antibodies against αMβ2 (CBP-α-αMβ2), αM (CBP-α-αM), and α3 (CBP-α-α3) de-differentiate myofibroblasts, lower the pro-fibrotic secretome of myofibroblasts, and reverse fibrosis in mouse lung and kidney models. These targets were generated by an mRNAseq study comparing monocytes cultured under anti- and pro-fibrotic conditions [44]. These results raise the possibility of using targeted anti-integrin antibodies as an immunotherapy against fibrosis.

### B. Results

The inventors began this study with the observation that monocyte differentiation into myofibroblasts was dependent on adhesion to a stiff surface [44]. The inventors sought to identify key pathways of monocyte-to-myofibroblast differentiation by performing an mRNAseq comparison of monocytes cultured on soft (1 kPa; not allowing myofibroblast differentiation) and stiff (12 kPa; allowing myofibroblast differentiation) surfaces (Table 1). The inventors identified a key role for integrins α3, αM, and αMβ2, and blocking antibodies against these integrins de-differentiated existing myofibroblasts and reversed fibrosis in a murine model of pulmonary fibrosis.

The mRNAseq study of monocytes cultured on soft (1 kPa) and stiff (12 kPa) surfaces revealed that integrins αM (ITGAM), α3 (ITGA3), and α7 (ITGA7) were directly upregulated by culture on the stiff surface (Table 1). Upstream regulators of β1 (ITGB1), β2 (ITGB2), and αX (ITGAX) were also upregulated. Interestingly, no TGFβ-specific integrin or upstream regulator was found to be upregulated, including ITGAV, ITGA11, ITGB3, ITGB5, ITGB6, ITGB7 [8], nor was TGFβ itself. Additionally, several upregulated pathways and functional annotation clustering also indicated the involvement of integrins in the differentiation of myofibroblasts (Table 1).

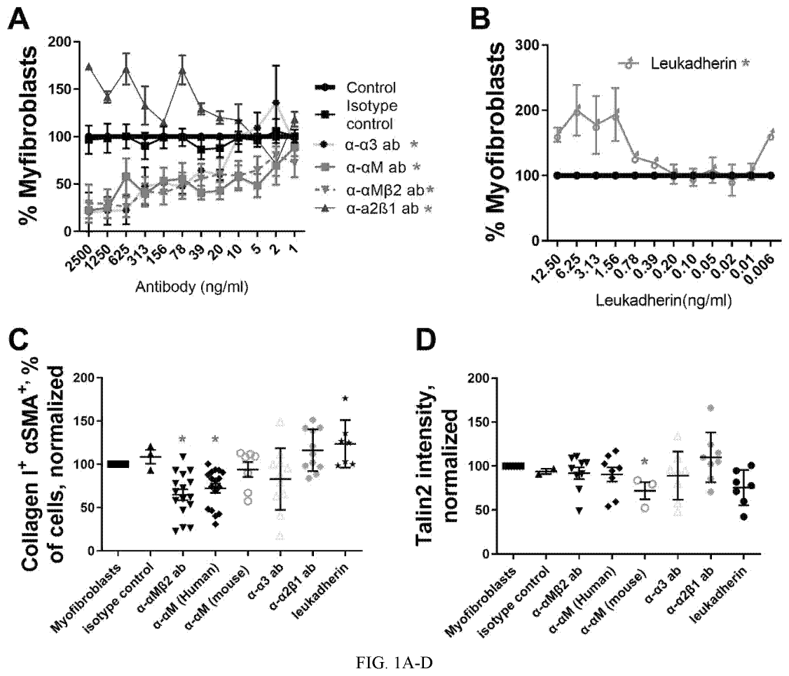

To confirm the results of the mRNAseq study, the inventors assessed the ability of anti-integrin antibodies to promote or inhibit myofibroblast differentiation. Blocking antibodies against α3 (α-α3), αM (α-αM), and against the heterodimer of αM and 32 (α-αMβ2) consistently de-differentiated monocyte-myofibroblasts (as determined by morphology) when added to culture (FIG. 1A, IC50 in Table 2), while α-α7, α-β1, α-αX inconsistently de-differentiated monocyte-myofibroblasts, and α-β2 caused apoptosis (data not shown, [45]). An antibody that stabilizes the association of integrin α2β31 (clone Gi14) promoted myofibroblast differentiation (FIG. 1A, [46]). The small molecule leukadherin, which increases the associated of αM and β2, also promoted myofibroblasts differentiation (FIG. 1B, [41]). Taken together, these results show that the mRNAseq study revealed key integrins which can govern myofibroblasts differentiation and de-differentiation, when their ligand binding is promoted or inhibited.

To determine if the inventors could reduce intracellular markers of myofibroblast differentiation, the inventors added α-α3, α-αM, and α-αMβ2 to monocyte-derived myofibroblasts and measured the number of αSMA⁺ collagen I⁺ double positive cells via flow cytometry. The IC50 from FIGS. 1A and 1B was used to generate an effective dose of 500 ng/ml antibody and 2 ng/ml leukadherin. 500 ng/ml α-αM and α-αMβ2 reduced αSMA⁺ collagen I⁺ cells from myofibroblast precursors, indicating that de-differentiation of monocyte-myofibroblasts was not limited to morphological changes (FIG. 1C). Similarly, both leukadherin and the integrin α2β1 stabilizing antibody increased the amount of αSMA⁺ collagen I⁺ double positive cells (FIGS. 1B and 1C).

In a companion study, the inventors showed that adhesion to a surface of sufficient stiffness (>12 kPa) is essential for monocyte-myofibroblast differentiation, and that inhibition of the stiffness-sensing spring protein talin2 de-differentiated monocyte-derived myofibroblasts and reversed existing lung fibrosis in a mouse model [44]. Neither integrin blockers (α-α3, α-αM, and α-αMβ2) nor integrin interaction promoters (Gi14, leukadherin) significantly altered talin2 expression in monocyte-derived myofibroblasts (FIG. 1D). Thus de-differentiation of monocyte-derived myofibroblasts by blocking antibodies (α-αMβ2, α-αM, α-α3) does not appear to operate through inhibition of talin2.

Myofibroblasts contribute to the development and maintenance of scar tissue in several distinct ways: by directly secreting ECM components (including collagen I), by using spindle-shaped morphology and cytoskeletal structures (FAs and FBs) to increase tissue rigidity, and by secreting pro-fibrotic cytokines and chemokines.

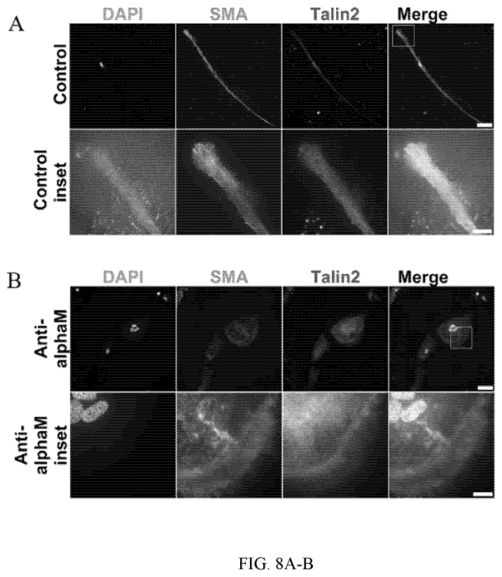

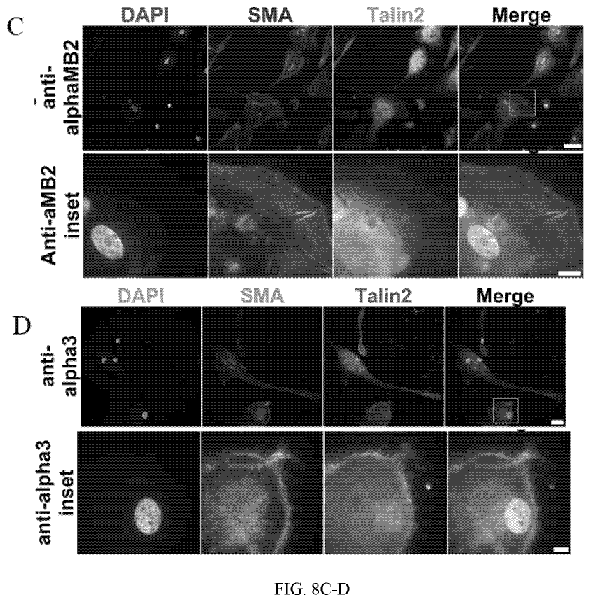

To determine if de-differentiation is accompanied by a loss of intracellular structures found in myofibroblasts (FAs and FBs), the inventors added 500 ng/ml α-α3, α-αM or α-αMβ2 to cultured monocyte-derived myofibroblasts. α-α3, α-αM or α-αMβ2 eliminated myofibroblast's spindle-shaped morphology and caused myofibroblasts to adopt a more macrophage-like morphology (FIG. 8A vs B-D). α-α3, α-αM and α-αMβ2 also induced the complete loss of myofibroblast cytoskeletal structures (FAs and FBs, FIG. 8B-D).

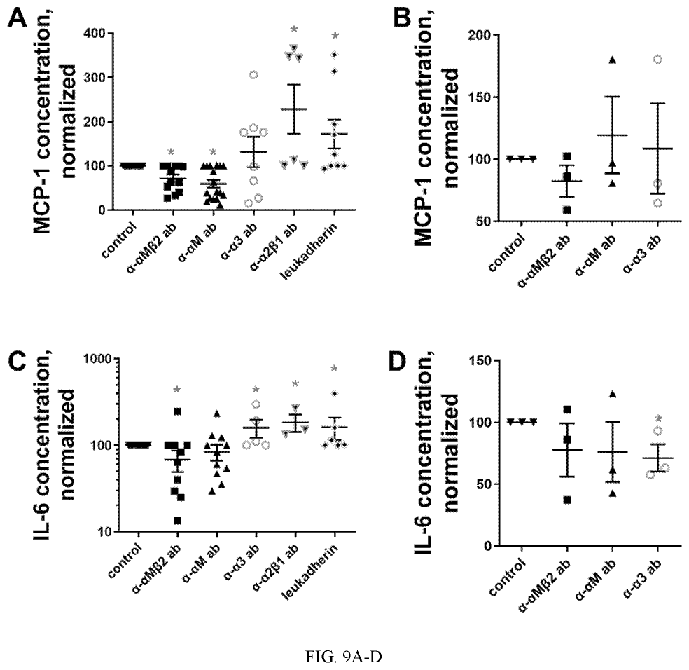

To determine if myofibroblast de-differentiation is accompanied by a change in secretome, the inventors added 500 ng/ml α-α3, α-αM or α-αMβ2 to monocyte-derived-myofibroblasts. α-αM and α-αMβ2 reduced the amount of secreted pro-fibrotic macrophage-chemotactic protein-1 (MCP1) from monocyte-derived myofibroblasts (FIG. 9A). α-αMβ2 also inhibited the amount of secreted IL-6 (FIG. 9C). Antibody clone Gi14 (which stabilizes the association of integrin α2β1) and leukadherin (which stabilizes integrin αMβ2) both promoted the secretion of MCP1 (FIG. 9A) and IL-6 (FIG. 9C) in monocyte-derived myofibroblasts, again showing that modulation of specific integrins can both promote or de-differentiate myofibroblast morphological phenotype and secretome of myofibroblasts.

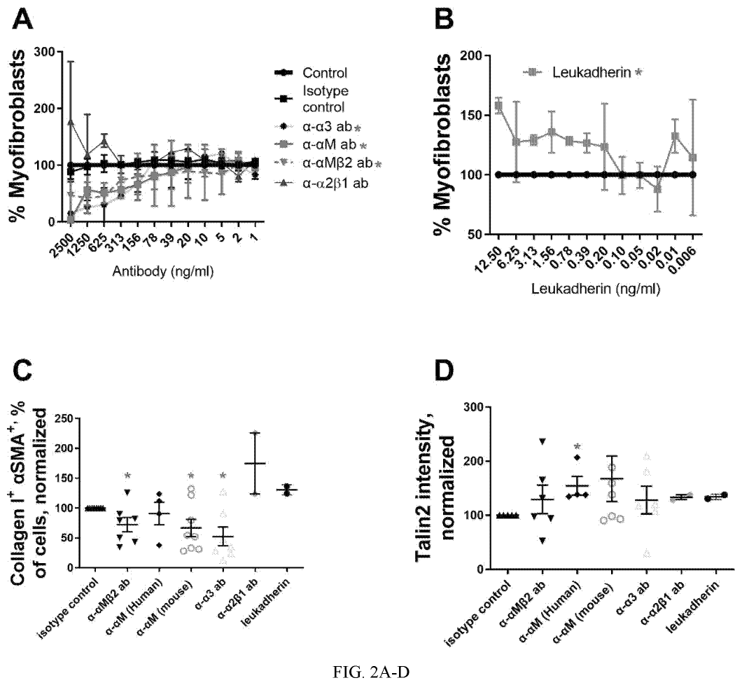

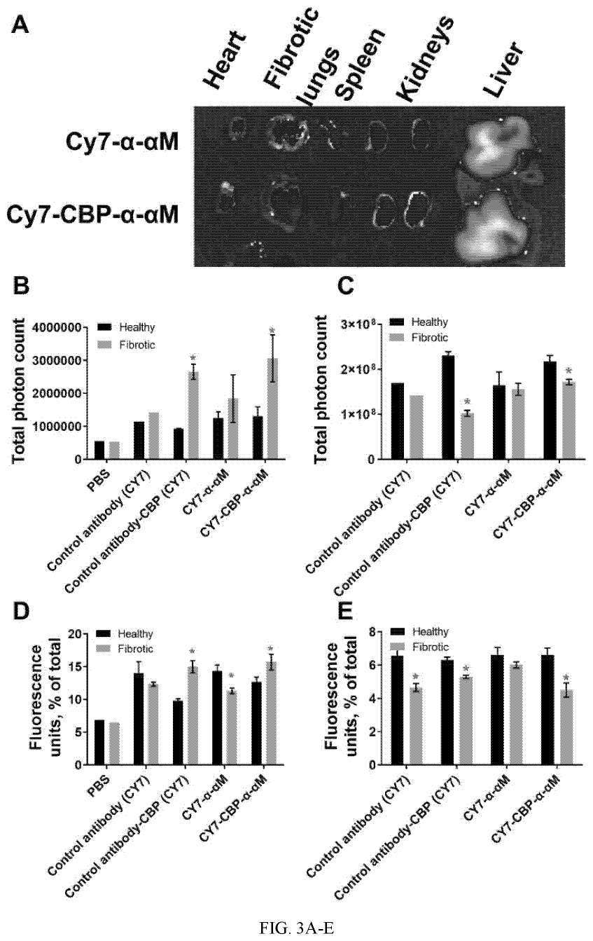

To determine if modulation of integrins could de-differentiate mouse myofibroblasts, the inventors added α-α3, α-αM, α-αMβ2, α-α2β1 (Gi14) and leukadherin to mouse monocyte-service myofibroblasts and mouse fibroblast-derived myofibroblasts. Blocking antibodies cross-reactive against mouse integrins α3, αM, αMβ2 (α-α3, α-αM, α-αMβ2) de-differentiated mouse monocyte-derived myofibroblasts (FIG. 2A).

Promoting the association of integrin α2β1 (via Gi14) and αMβ2 (via leukadherin) both increased monocyte-derived myofibroblast differentiation (FIGS. 2A and B). α-α3, α-αM, α-αMβ2 also reduced the number of αSMA⁺ and collagen I⁺ double positive myofibroblasts (FIG. 2C), and did not decrease the amount of talin2⁺ cells (FIG. 2D). This again suggests that de-differentiating myofibroblasts by blocking α3, αM, αMβ2 operates through a different mechanism than de-differentiating myofibroblasts by inhibition of talin2. Stabilizing integrin α2β1 (through Gi14) and αMβ2 (via leukadherin) increased both the amount of α αSMA⁺ and collagen I⁺ double positive monocyte-myofibroblasts, as well as increasing talin2 concentration in these cells (FIGS. 2C and 2D).

Antibody CBRM1/5 is raised against the activation epitope of human αMβ2 [47], but shares 2 amino acid (AA) overlap with mouse αMβ2 [48]. However, CBRM1/5 de-differentiates mouse monocyte-derived myofibroblasts (FIG. 2A), though at a reduced effectiveness vs human monocyte-derived myofibroblasts (FIG. 1A). While it would have been ideal to use an anti-mouse αMβ2 antibody, no such antibody exists raised against the active conformation of mouse αMβ2.

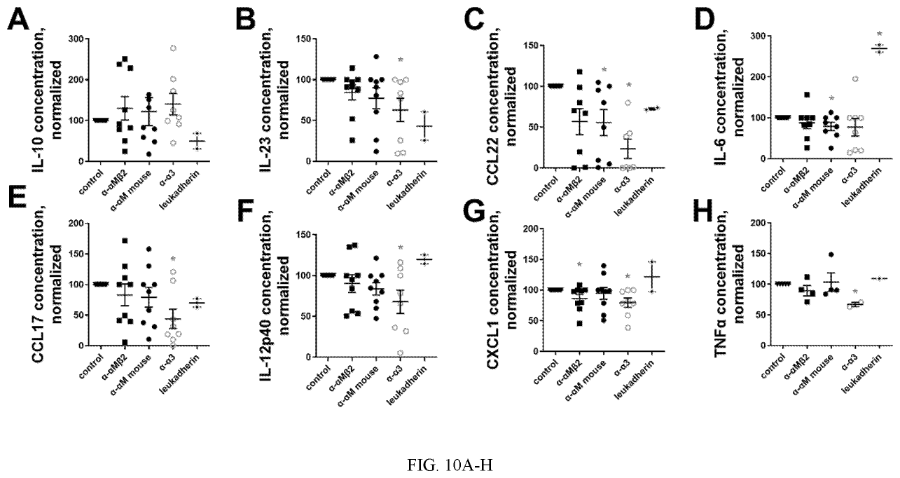

To determine if de-differentiation of mouse myofibroblasts is accompanied by an altered secretome, the inventors assessed the conditioned media from mouse myofibroblasts treated with 500 ng/ml α-α3, α-αM, and α-αMβ2. De-differentiation of myofibroblasts did not alter the amount of secreted anti-fibrotic IL-10, but in some cases significantly reduced (and did not increase in any case) the amount of pro-fibrotic IL-23 [49], CCL22 [50], IL-6 [51], CCL17 [50, 52], IL-12 subunit p40 [53], CXCL1 [54], and TNF-α [55](FIG. 10).

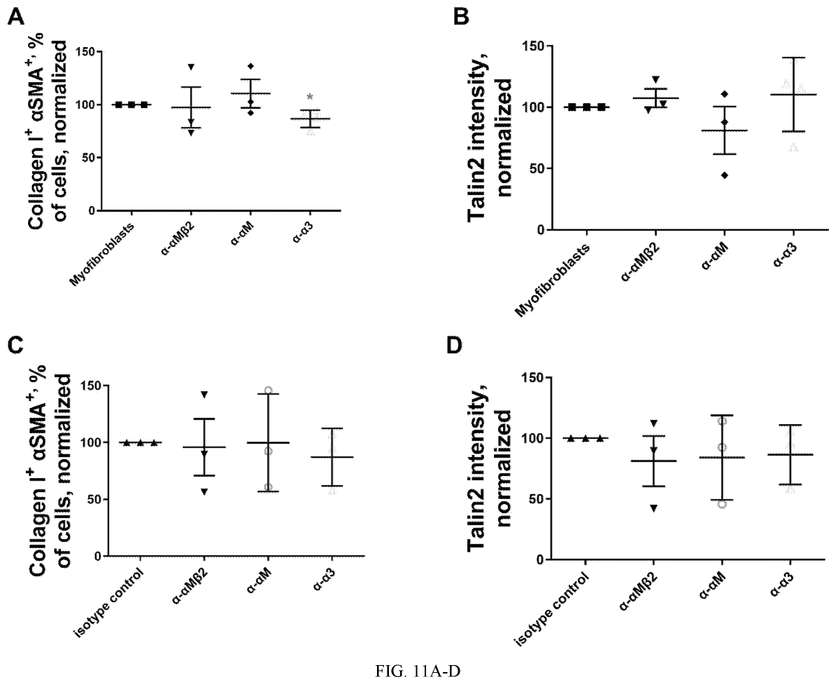

While monocytes can become myofibroblasts, the primary cellular component of scar tissue in fibrosis is the fibroblast-derived myofibroblast. To determine if fibroblast-derived myofibroblasts could be de-differentiated, the inventors added 500 ng/ml α-αMβ2, α-αM, α-α3 to fibroblast-derived myofibroblasts and measured the number of αSMA⁺ and collagen I⁺ double positive cells, and talin2⁺. α-α3 reduced the number of αSMA⁺ and collagen I⁺ fibroblast-derived myofibroblasts, while α-αM and α-αMβ2 did not (FIG. 11A), consistent with the expression of these integrins on monocytes but not fibroblasts. Neither α-αMβ2, α-αM, α-α3 treatment lowered the amount of talin2 (FIG. 11B), again confirming that de-differentiation of myofibroblasts by inhibiting integrin binding and inhibiting tension sensing operate by non-overlapping mechanisms. α-α3 decreased the amount of IL-6 secreted from fibroblast-derived myofibroblasts (FIG. 9D). No antibody treatment lowered the number of mouse αSMA⁺ and collagen I⁺ double-positive fibroblast-derived myofibroblasts, or talin2⁺ (FIGS. 11C and D).

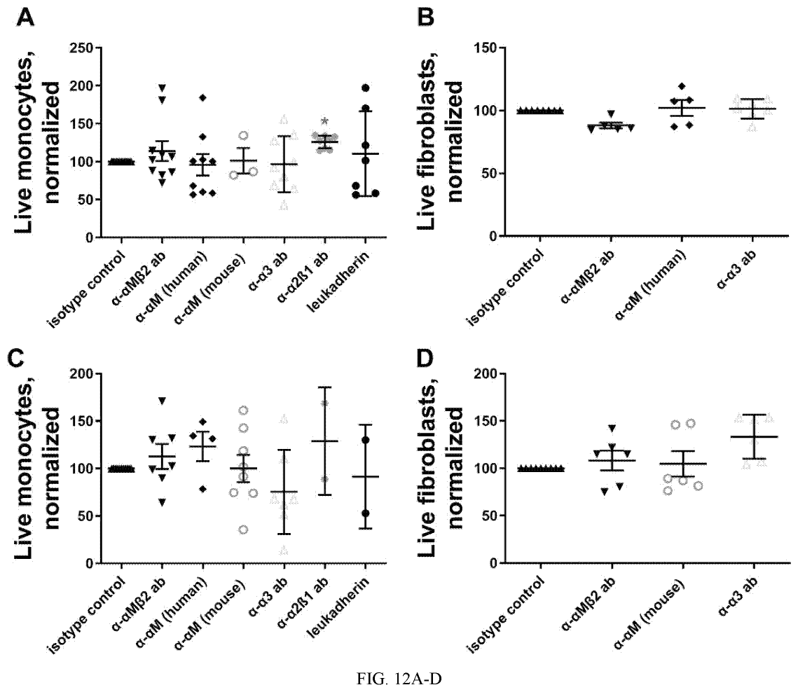

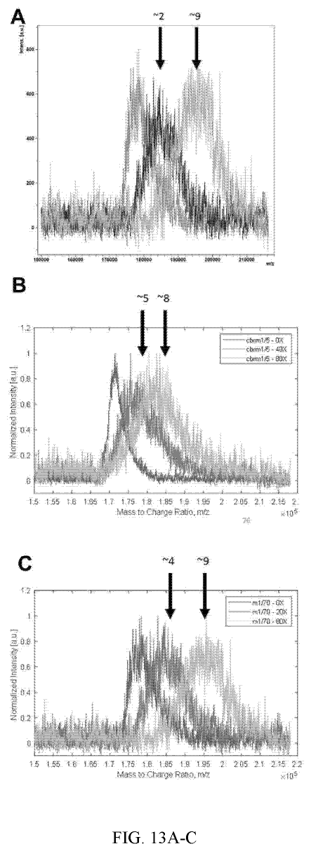

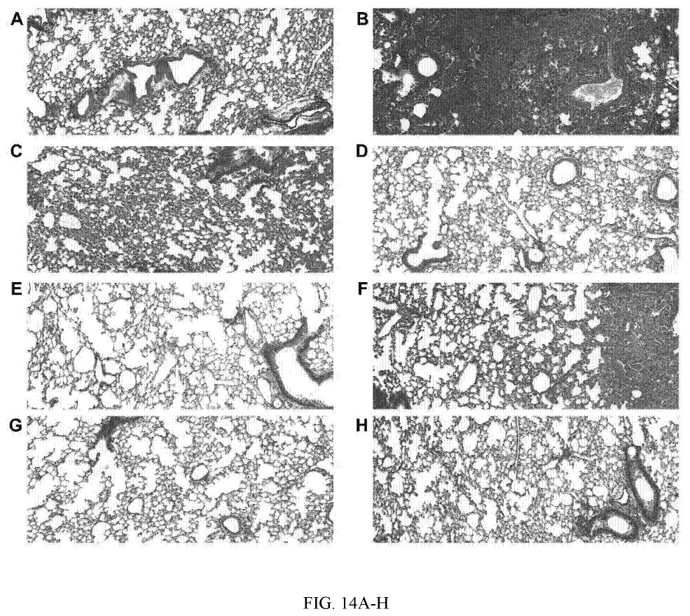

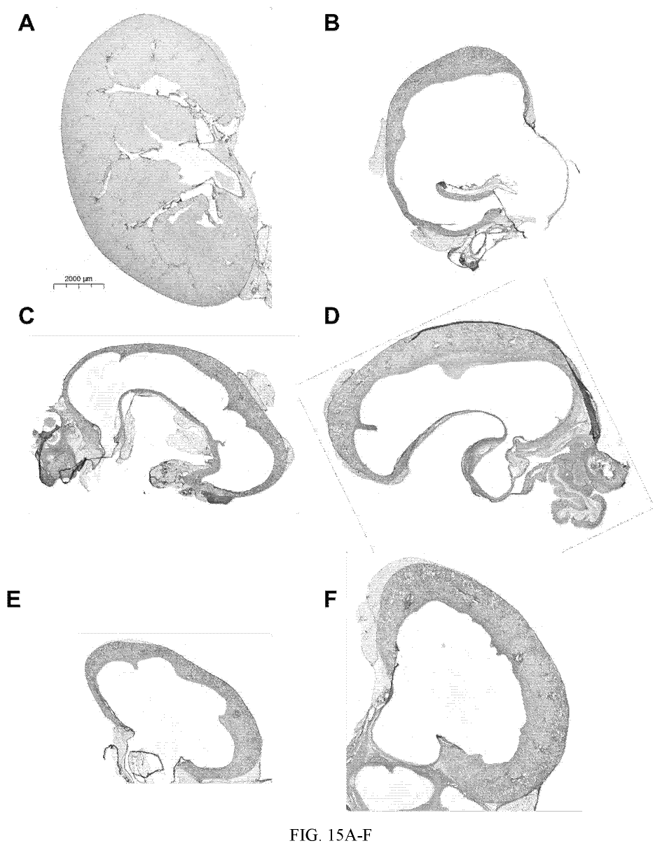

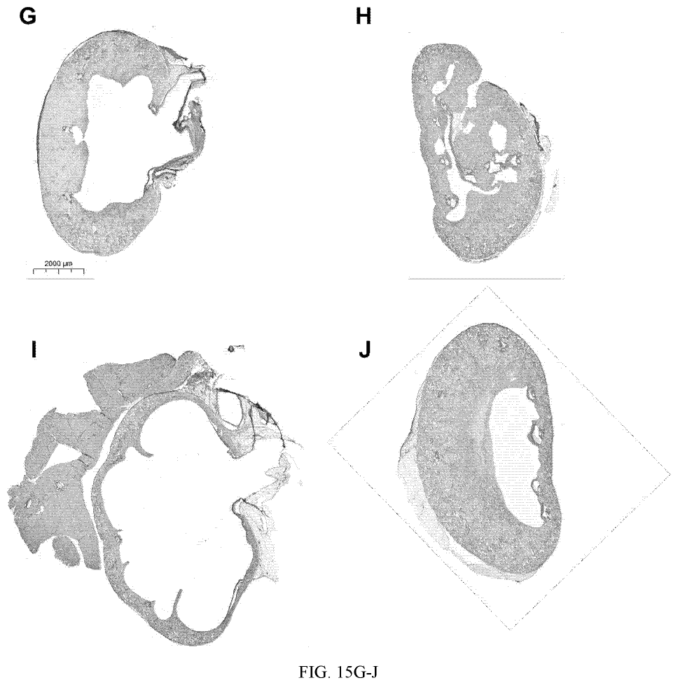

Treatment with antibodies did not induce cell death from monocyte (FIGS. 12A and C) or fibroblast populations (FIGS. 12B and D), among both human and mouse cells. This confirms that the de-differentiation of myofibroblasts is not caused by, or accompanied by, an increase in cell death.

Conjugation of decorin's collagen-binding peptide (CBP) to an antibody increased the proportion of the conjugated CBP-antibody that reached and was retained in fibrotic lungs, compared to non-conjugated antibody [43]. This finding involved antibodies against soluble factors (α-TNFα and α-TGFβ). To determine if the inventors could deliver an anti-integrin antibody to a fibrotic organ, the inventors conjugated CY7-CBP to α-αM. The inventors instilled mouse lungs with bleomycin, allowed fibrosis to develop over a week, and injected

Cy7-CBP-α-αM and Cy7-α-αM i.v. in mice with healthy and fibrotic lungs. The inventors compared the fluorescence of the harvested organs after 48 hr via IVIS (FIG. 3A). Direct comparison of the fluorescence of healthy and fibrotic lungs (FIG. 3B) shows that significantly more fluorescently labeled antibody remained in fibrotic lungs after 48 hr. Because αM is present on monocytes, and monocytes are enriched in the spleen, the inventors compared the fluorescence of spleens (FIG. 3C). There was significantly less Cy7-CBP-α-αM in the spleen in animals with fibrotic lungs, suggesting that the enrichment of Cy7-CBP-α-αM in the lungs was coming partially at the expense of Cy7-CBP-α-αM in the spleen.

To make certain that the Cy7-CBP-α-αM is not simply remaining in circulation in the mouse for longer, the inventors pooled the total fluorescence of all organs for Cy7-CBP-α-αM and CY7-CBP conjugated isotype control antibody, and normalizing the fluorescence for each organ. This analysis showed significantly more Cy7-CBP-α-αM in fibrotic lungs than Cy7-α-αM in fibrotic lungs or Cy7-CBP-α-αM in healthy lungs (FIG. 3D) and less in the spleen (FIG. 3E).

To determine if the inventors could reverse existing fibrosis in addition to de-differentiating myofibroblasts, the inventors conjugated non-fluorescent CBP to each of α-α3 (CBP-α-α3), α-αM (CBP-α-αM), and α-αMβ2 (CBP-α-αMβ2). Using the recently published results as a guide [43], the inventors used different molar excess of CBP to establish ratios sufficient to attach at least 5 CBP peptides (on average) to each antibody (FIG. 13).

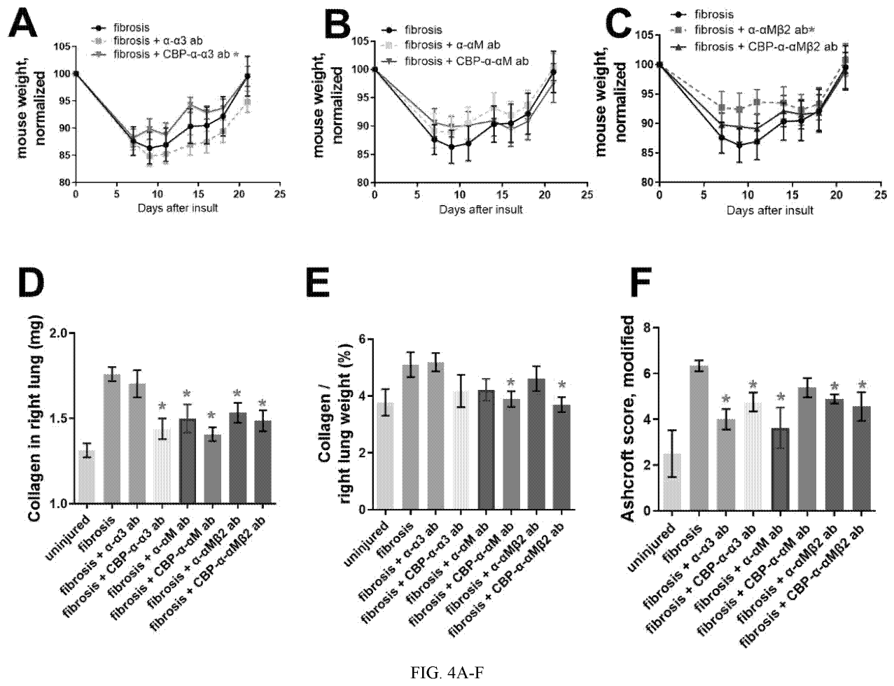

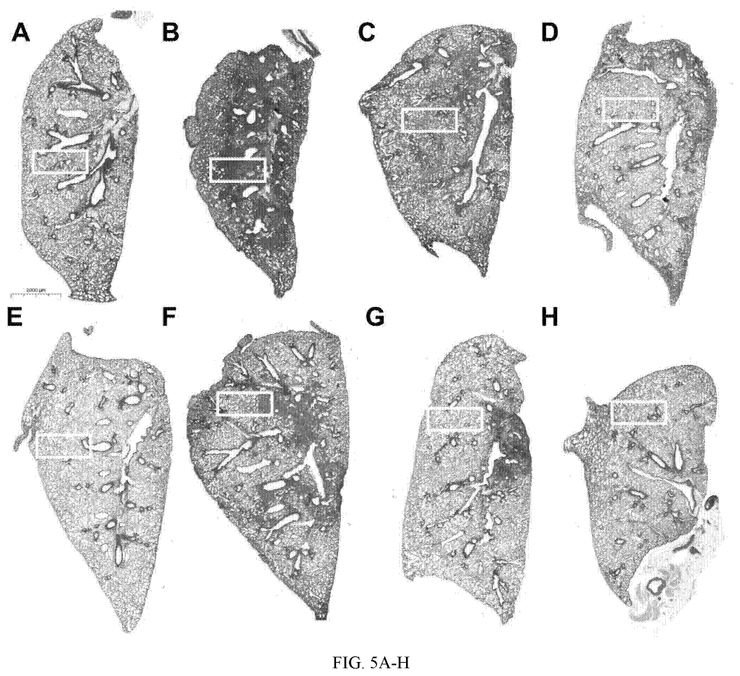

The inventors instilled mouse lungs with bleomycin, allowed fibrosis to develop, and injected α-α3, α-αM, α-αMβ2, CBP-α-α3, CBP-α-αM, and CBP-α-αMβ2 at 7, 9, 11, 14, 16, and 18 days post bleomycin insult. The mice were euthanized 21 days post insult. Only CBP-α-α3 and α-αMβ2 provided weight gain that was statistically higher than the no-treatment control (FIG. 4A-C). CBP-α-α3, α-αM, CBP-α-αM, α-αMβ2, and CBP-α-αMβ2 significantly reduced the total amount of collagen in the right lobe of the lung vs the untreated fibrotic lungs, as assessed by hydroxyproline assay (FIG. 4D). CBP-α-αM and CBP-α-αMβ2 significantly reduced the amount of collagen in the right lungs as a percentage of overall lung weight, compared to the untreated fibrotic lungs (FIG. 4E).

The left lobes of the bleomycin-insulted lungs were mounted in paraffin, sectioned, stained using Masson's trichrome, and the histology was blindly scored using the modified Ashcroft method [56] by a researcher not involved in administration of the treatment (FIG. 4F). Both α-α3 and CBP-α-α3 reduced the Ashcroft score, as did both α-αMβ2 and CBP-α-αMβ2; with the α-αMβ2 antibody, only the unmodified antibody showed a reduction (FIG. 4F-G). Representative images of the Masson's trichrome stained left lobes can be seen in FIG. 5 A-H.

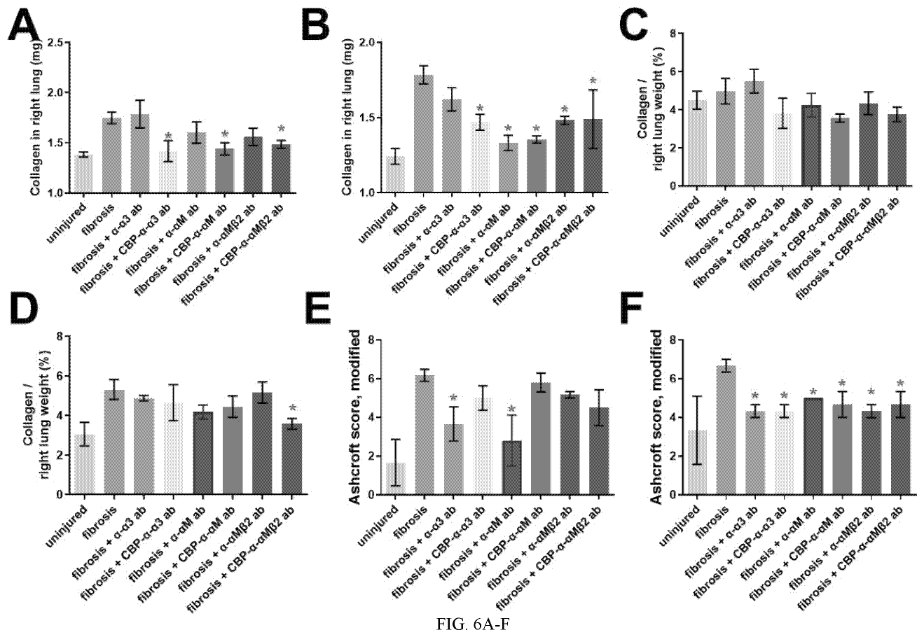

To determine if treatment by α-α3, α-αM, and α-αMβ2 and CBP-α-α3, CBP-α-αM, and CBP-α-αMβ2 improved fibrosis in both male and female mice, the inventors analyzed the hydroxyproline-based quantitative collagen data (FIG. 6A-D) and histology-based Ashcroft qualitative data (FIG. 6E-H) based on mouse sex. In general, in this model males show a more fibrotic response. Treatment by α-α3, α-αM, and α-αMβ2 and CBP-α-α3, CBP-α-αM, and CBP-α-αMβ2 reduced the absolute amount of collagen in the right lobes of both male and female mice (FIG. 6A-B), though reductions in the amount of collagen as a portion of lung weight were not significant except in the case of CBP-α-αMβ2 in female mice (FIG. 6C-D). Several treatments improved the modified Ashcroft scores for both male and female mice (FIG. 6EH).

Overall, these data indicate that treatment of fibrosis with blocking anti-integrin antibodies directed against α3 and αMβ2 can reverse pulmonary fibrosis after a bleomycin insult.

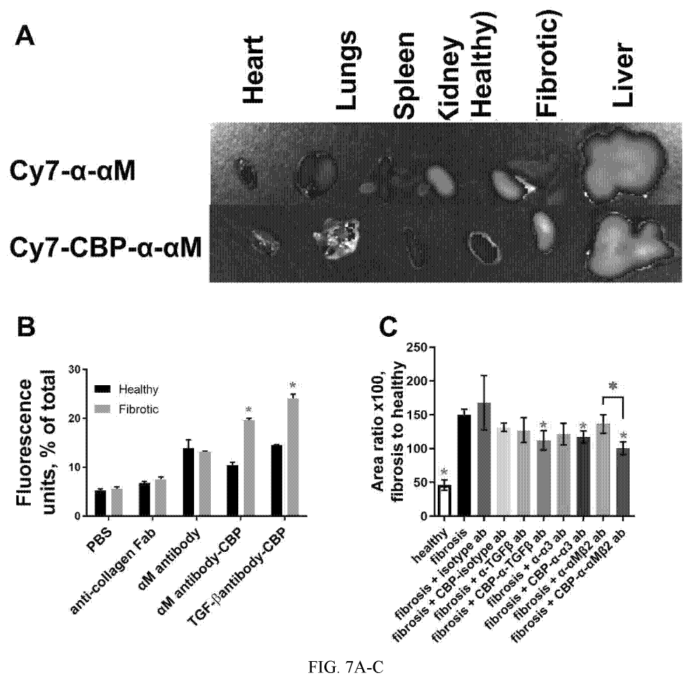

To determine if the inventors could reverse existing kidney fibrosis in addition to existing lung fibrosis, the inventors attempted to target CBP-tagged antibodies to fibrotic kidneys, similar to FIG. 3 for lungs. The inventors ligated the descending ureter of the left kidney (unilateral ureteral obstruction, UUO) and allowed the kidneys to become damaged for 1 week. The inventors then injected Cy7-CBP-α-αM and Cy7-α-αM i.v. The inventors compared the fluorescence of the harvested organs after 48 hr via IVIS (FIG. 7A). As in FIG. 3, to make certain that Cy7-CBP-α-αM is not simply remaining in circulation in the mouse for longer, the inventors pooled the total fluorescence of all organs for Cy7-CBP-α-αM and CY7-α-αM, and normalizing the fluorescence for each organ. This analysis showed significantly more Cy7-CBP-α-αM in fibrotic kidneys than in healthy kidneys, in comparison to the ratio for Cy7-α-αM (FIG. 7B). The inventors also tested the targeting potential of Cy7-CBP-α-TGFβ, which the inventors had previously shown to be anti-fibrotic when targeted to lungs (FIG. 7B) [43].

These results indicate the inventors can target the antibodies to fibrotic kidneys, and in greater quantity than to fibrotic lungs.

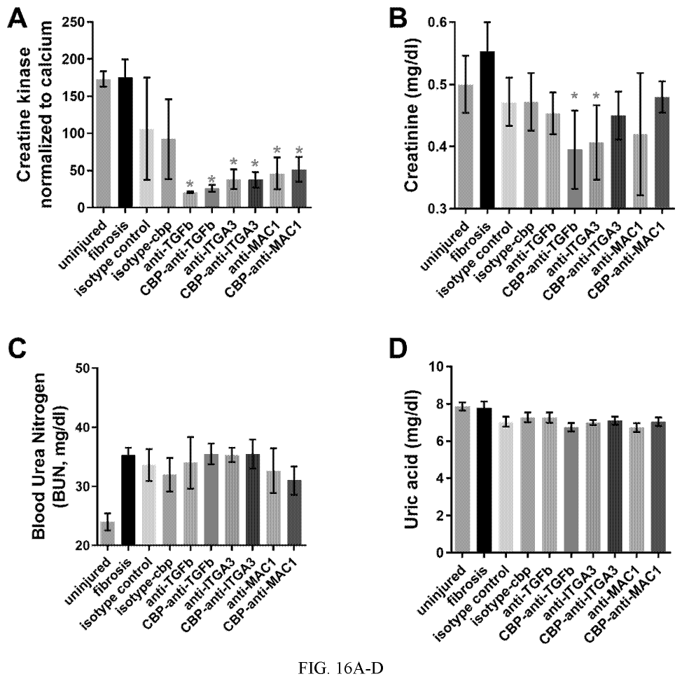

To determine if CBP-α-TGFβ, CBP-α-α3, and CBP-α-αMβ2 could reverse existing kidney fibrosis, the inventors performed a UUO on the left kidney. At 1 week post-UUO the inventors injected 50 micrograms of α-TGFβ, α-α3, α-αMβ2, CBP-α-TGFβ, CBP-α-α3, and CBP-α-αMβ2 i.v. At 2 weeks post-UUO, the inventors resected the kidneys, and assessed fibrosis via immunohistochemistry (IHC) for collagen I (FIG. 7, FIG. 15) and blood markers of kidney injury (FIG. 16). CBP-α-TGFβ, CBP-α-α3, and CBP-α-αMβ2 lowered the amount of collagen present in the UUO kidneys (FIG. 15, FIG. 7C). An analysis of blood taken from the kidney

UUO mice indicates that each antibody treatment reduced the amount of circulating creatine kinase, but did not change the concentration of blood urea nitrogen (BUN) or uric acid (FIG. 16). This could be because the UUO model leaves mice with one entirely healthy kidney. These results indicate a single 50 microgram dose of CBP-α-TGFβ, CBP-α-α3, and CBP-α-αMβ2 is capable of partially reversing kidney fibrosis.

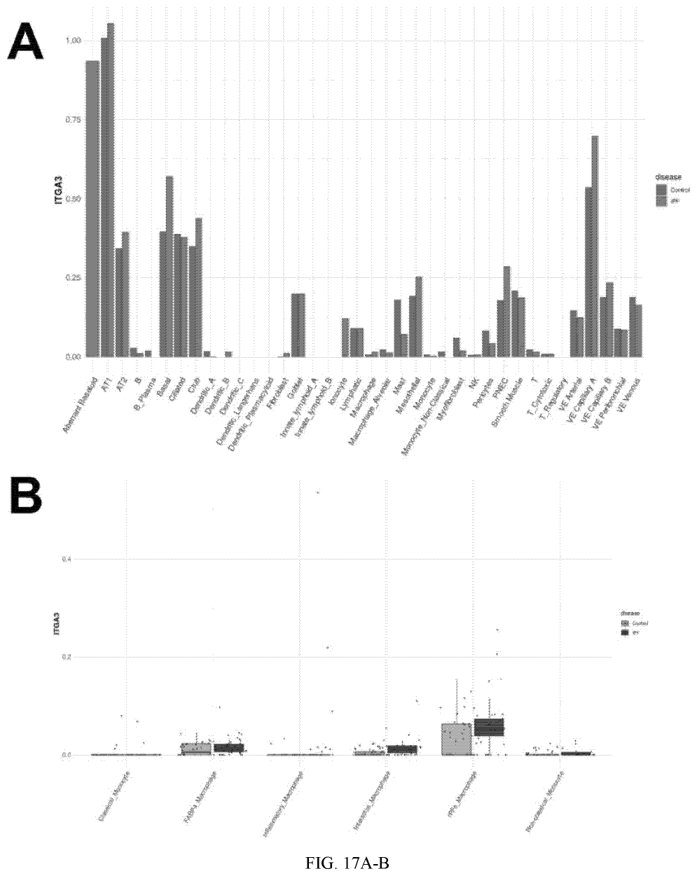

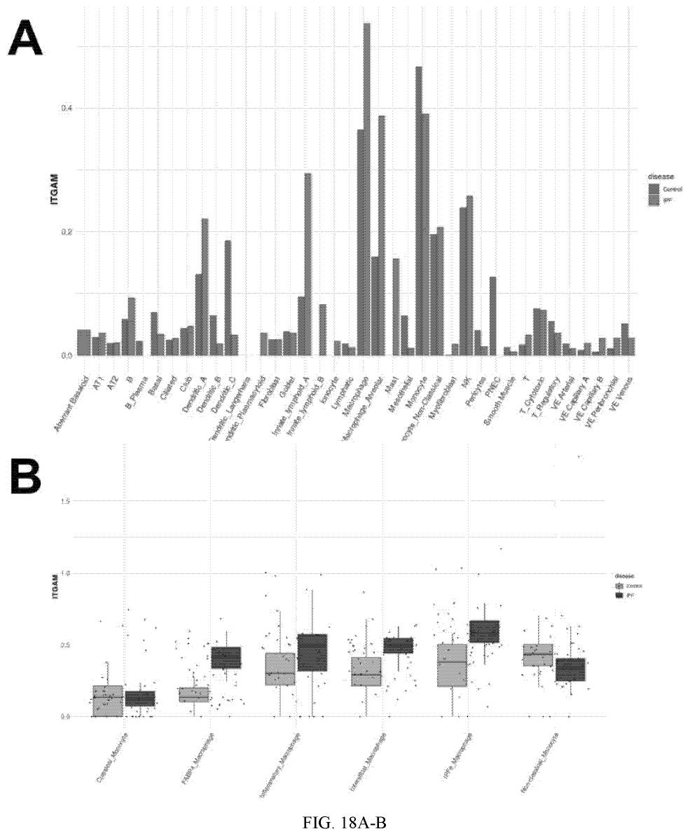

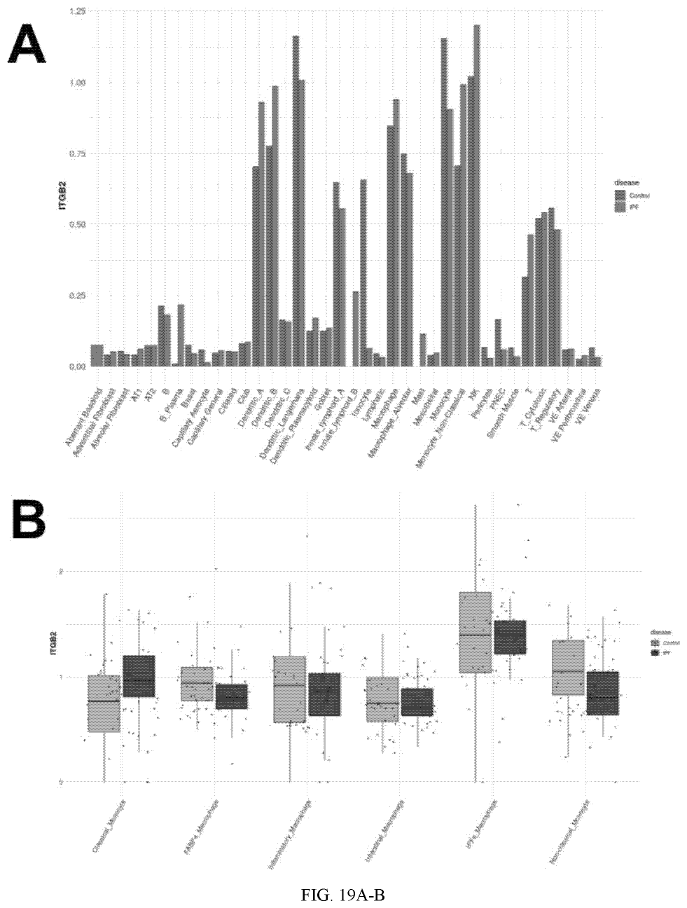

In summary, α-α3, α-αM, and α-αMβ2 were identified as proteins upregulated during the differentiation of monocytes into myofibroblasts. Blocking antibodies against α-α3, α-αM, and α-αMβ2 reverse myofibroblast differentiation and reduce the pro-fibrotic secretome of myofibroblasts. Injected CBP-conjugated antibodies preferentially localize at fibroses in both the lungs and the kidneys, and CBP-conjugated blocking antibodies (CBP-α-TGFβ, CBP-α-α3, CBP-α-αM, and CBP-α-αMβ2) reverse lung and kidney fibroses at lower doses than untargeted antibodies. Transcripts for integrins α3, αM, and β2 are each upregulated in macrophages in idiopathic pulmonary fibrosis (IPF) (FIGS. 17, 18, and 19, data from IPF cell atlas [57]). These results raise the possibility of a targeted immunotherapy treatment for fibrosis.

### C. Discussion

The overall objective in this work is to explore an approach by which to de-differentiate myofibroblasts and reverse fibrosis. This study was guided by protein targets identified from an RNAseq comparison of myofibroblasts under anti- and pro-fibrotic culture conditions related to surface stiffness (1 kPa and 12 kPa) [44]. These targets were subsequently validated and refined by in vitro experimentation, where only blocking antibodies α-αMβ2, α-αM, α-α3 consistently de-differentiated myofibroblasts (FIGS. 1, 2, and 8-10). Each of α-αMβ2, α-αM, and α-α3 reversed existing fibrosis in mouse models of lung and kidney fibrosis (FIG. 4-7).

Myofibroblast de-activation has long been a goal of fibrosis research. De-activation can be achieved by myofibroblast apoptosis [14] or de-differentiation [9]. Since no apoptosis was observed by immunofluorescence (FIG. 8B-D) and no increase in live-dead staining was observed after treatment with α-α3, α-αM, or α-αMβ2 (FIG. 12), the inventors conclude the inventors have de-activated myofibroblasts through de-differentiation. De-activating monocyte-derived myofibroblasts by de-differentiation seems preferable to apoptosis, since monocyte-derived cells are proficient at removing deposited ECM and regenerating tissue, both of which are required to reverse fibrosis [7].

While the inventors showed in this paper that targeting the αMβ2 complex and αM both de-differentiate myofibroblasts and reverse fibrosis, targeting β2 (CD18) with blocking antibodies resulted in apoptosis of myofibroblasts [45](Data not shown), which rendered this blocking antibody unsuitable for use in this study. However, this reported result may be an example of de-activation of myofibroblasts by apoptosis.

While the inventors have shown that α-αM and α-αMβ2 reverse existing fibrosis, some evidence indicates they may have a protective role against fibrosis as well. α-αM is effective in reducing tissue damage in a kidney-ischemia model [58], while α-αMβ2 antibody prevented both damage from kidney-ischemia and subsequent ischemia-induced fibrosis [59].

Only α-α3—and not α-αM or α-αMβ2—de-differentiated fibroblast-derived myofibroblasts (FIG. 11). While fibroblasts express integrin α3, the integrins αM and αMβ2 are considered canonical myeloid markers. Thus, it appears that anti-integrin antibodies are sufficiently specific that different lineages of myofibroblasts can be targeted by anti-integrin antibodies to reverse fibrosis.

Hinz has proposed the “super-mature” FAs are essential for the development and maintenance of the myofibroblast phenotype [9]. Super-mature FAs concentrate in one location several different protein components of cell adhesion and tension sensing: integrins, tension-sensing talins, and cytoskeletal machinery [31, 32]. FAs participate in inside-out and outside-in signaling in relation to their size [60]. FIG. 8A (inset) shows structures remarkably similar to Hinz's super-mature FAs [29], and images in FIGS. 8B, C and D show that treatment of myofibroblasts with α-αMβ2, α-αM, and α-α3 both remove both the myofibroblast morphology and the presence of FAs and FBs. That removal of the myofibroblast morphology occurs in parallel with the removal of FAs and FBs perhaps confirms Hinz's proposal that super-mature FAs are critical structures for the maintenance of myofibroblasts [9].

αMβ2 is necessary for maintenance of monocyte binding, allowing the actin reorganization that sustains adhesion [61]. This suggests that disruption of αMβ2 might remove existing FAs, again recalling Hinz's hypothesis that FAs are critical to myofibroblast differentiation and maintenance. Treatment of monocyte-derived myofibroblasts with α-αMβ2, α-αM and α-α3 induced a morphology (FIG. 8B-D) that appears similar to fluorescence images from monocytes [62] and fibroblasts [63, 64] that have had talin2 reduced, suggesting that integrin-blocking antibodies and inhibition of talin2 both might de-differentiate myofibroblasts through disruption of existing FAs.

Talins and integrins interact in multiple ways within FAs. Talin's interaction with the β-tail of integrins [24] is essential for “outside-in” signaling of integrins [65]. Talin2's affinity for the β-tail of β2 increases the binding of monocytes to cells in two key examples. First, talin2's interaction with β2 promotes αLβ2 adhesion to ICAM-1 [66]. Second, talin2 is also essential for αMβ2-mediated phagocytosis [62]. The differences in tension-sensing and mechanotransduction between talin1 and talin2 may be primarily due to each isoform's respective integrin associations [25], suggesting the interaction of talins and integrins may play a large role in tension sensing. Intriguingly, integrin-dependent mechanosensing is talin isoform specific [64]. The difference in talin's affinity for each integrin [25], the subcellular localization of integrins and talins, and interactions with the adhesome are all part of the dynamic mechanosensing and mechanotransduction of monocytes during myofibroblast differentiation [8].

While integrin interactions with tension-sensing talins appears to be key to the formation and maintenance of myofibroblasts, integrins themselves may play a role in tension sensing. The speed at which integrin-actin linkages are formed and broken has been suggested to be a cellular tension sensing mechanism in and of itself [33, 67-69]. This suggests that integrins may transduce information about specific binding ligands (including ECM components) and surface stiffness. This may explain how several integrins appeared in a screen designed to assess the differences in cultured monocytes between soft (1 kPa) and stiffer (12 kPa) surfaces, on the stiffer surfaces permitting monocyte-to-myofibroblast differentiation.

De-differentiation of mouse and human myofibroblasts resulted in strongly reduced IL-6 and MCP-1 secretion (FIGS. 9 and 10). Removing the MCP-1 pathway abrogates kidney injury [70], again suggesting monocyte-derived cells are quite important in fibrosis. IL-6 is quite pro-fibrotic, in some cases sufficient to induce myofibrobasts [71]. That MCP-1 and IL-6 were the two consistently inhibited secreted proteins in the cytokine screens suggests that de-differentiation of monocyte-derived myofibroblasts may have additional effects beyond the de-activation of individual myofibroblasts, and may contribute to an overall reduction in the pro-fibrotic environment in general.

Several anti-integrin therapies have failed to translate to medicinal use due to side-effects or lack of efficacy [24]. The hope was that by targeting antibodies to fibrotic tissue using the decorin-derived CBP (FIGS. 3 and 7), the inventors could increase the local concentration of anti-integrin antibodies in fibrotic organs, and potentially make anti-integrin antibodies a more useful therapeutic. The inventors further tested a monoclonal antibody that recognizes only the activation epitope of αMβ2 [47], clone CBRM1/5. This antibody binds only to a conformational epitope exposed on activated αMβ2. By using an antibody that recognizes only a conformational epitope, the inventors hope to further reduce the side-effects of anti-integrin treatments.

Each of α-α3, α-αM, and α-αMβ2 (and their CBP-conjugated variants) de-differentiated mouse myofibroblasts and reversed fibrosis in mice at an identical dose to previously published antibody therapeutics for fibrosis [43]. This indicates that the local concentration of untargeted α-α3, α-αM, and α-αMβ2 is sufficient to reverse fibrosis in mice. CBP-conjugated antibodies were twice as concentrated as unconjugated antibodies in fibrotic lungs after 48 hr (FIG. 3B) and in fibrotic kidneys after 24 hours (FIG. 7B). A single 50 microgram dose of CBP-conjugated antibodies was sufficient to partially reverse fibrosis in a kidney model of unreversed UUO kidney fibrosis (FIGS. 7 and 15).

These results show that α-α3, α-αM, and α-αMβ2 may be potentially useful as translational therapeutics, capable of bringing immunotherapy treatments to fibrosing diseases. The inventors again note that the α-αMβ2 clone used was less effective on mouse myofibroblasts (and thus presumably in the mouse model) than on human monocyte-derived myofibroblasts. Clinical attractiveness may be particularly high for CBP-α-αMβ2 (clone CBRM1/5), which both recognizes only the active ligand-binding configuration for αMβ2, and is also capable of being targeted to sites of fibrosis to increase the local concentration.

### D. Materials and Methods Study Design

This study was designed to test the strategy that key pathways of myofibroblast differentiation can be revealed by an RNAseq of myofibroblast precursors (monocytes) cultured on anti-fibrotic soft (1 kPa) and pro-fibrotic stiff (12 kPa) surfaces [44]. Specifically, this study examines whether antibodies against key integrins upregulated in monocyte-myofibroblasts (α-α3, α-αM, and α-αMβ2) can de-differentiate myofibroblasts and reverse fibrosis in a mouse model of lung fibrosis. CBP functionalization was employed to enhance retention in the fibrotic microenvironment.

Group size was selected based on experience with the pulmonary fibrosis model. Mice were randomized into treatment groups within a cage to eliminate cage effects from the experiment. Treatment was performed by multiple researchers over the course of this study, to ensure reproducibility. Lungs were also resected by multiple researchers, and blinded scoring was used on the fibrosis histology images.

### 1. Purification of Human Monocytes

All human blood was acquired through the University of Chicago blood donation center, in accordance with human subject protocol at the University of Chicago. In order to isolate more peripheral blood mononuclear cells (PBMCs) than is possible through a single blood donation, PBMCs were purified from leukocyte reduction filters from de-identified blood donors. Blood was filtered, and leukocytes purified, the same afternoon as a morning blood donation, to reduce the amount of time that PBMCs could adhere to the filter.

Leukocyte reduction filters were sterilized with 70% ethanol, and the blood flow tubes on either end were clamped shut. The tube through which filtered blood had exited the filter was cut below the clamp, and a syringe containing 60 ml phosphate buffered saline (PBS) was inserted into the tube. Following this, the tube through which unfiltered blood had entered the filter was unclamped, and PBS was slowly pushed through the filter in the opposite direction of the original blood flow. Reversing the flow of PBS through the filter resulted in a recovery of approximately 300 million cells per filter.

The collected blood was layered with lymphocyte separation media (LSM), and centrifuged at 1300×G for 20 min. The PBMC layer was then removed by pipetting.

Monocytes were purified from PBMCs by use of a negative selection kit for human monocytes (Stemcell, Cambridge, MA), per the manufacturer's instructions. Approximately 20 million monocytes were purified from each filter. Monocytes were then washed by PBS using five successive 300×G centrifugation steps, in order to remove EDTA from the resulting population. Monocytes were checked for purity using flow cytometry, and average purity was above 95%. Monocytes were cultured immediately following purification, at 100,000 monocytes/cm².

### 2. Purification of Mouse Monocytes

All mouse experiments were performed under supervision with protocols approved by the University of Chicago IACUC. Spleens were resected from healthy C57BL/6 mice, pooled, and were placed in PBS with 1 mM EDTA, 2% fetal calf serum (FCS) to prevent subsequent clumping of cells. All PBS, plasticware, filters, glassware and magnets were pre-chilled to 4 C, and kept cold throughout this procedure, to limit the clumping of cells. Importantly, ACK lysis buffer caused cell death, and so was not used in this procedure.

Spleens were pushed through a 100 m filter to disassociate the cells. Monocytes were purified from disassociated cells by use of a negative selection kit (Stemcell), following the manufacturer's instructions. The purified monocytes were then washed by PBS using five successive 300×G centrifugation steps, in order to remove EDTA from the resulting population. Monocytes were checked for purity using flow cytometry, and average purity was above 95%. The average yield was 1.5 million monocytes per spleen. Monocytes were cultured immediately following purification, at 250,000 monocytes/cm².

### 3. Culture of Human and Mouse Monocytes

Human and mouse monocytes were cultured as previously described, using serum-free media (SFM) [72]. Briefly, SFM for human cells is composed of fibrolife (Lifeline, Frederick, MD), with 1× ITS-3 (Sigma, St. Louis, MO), 1× HEPES buffer (Sigma), 1× non-essential amino acids (Sigma), 1× sodium pyruvate (Sigma), and penicillin-streptomycin with glutamate (Sigma). For mouse monocytes, 2× concentrations of ITS-3, HEPES buffer, non-essential amino acids, and sodium pyruvate were added, with 50 μM β-mercaptoenthanol (ThermoFisher) and pro-fibrotic supplements M-CSF (25 ng/ml, Peprotech, Rocky Hill, NJ) and IL-13 (50 ng/ml) to induce myofibroblast differentiation. Additionally, M-CSF and IL-13 were refreshed in the media of mouse monocytes after 3 days of culture. Monocytes were allowed to differentiate for 5 days, and counted based on morphology as previously described [73].

Myofibroblasts were de-differentiated by addition of antibodies (α-αMβ2, α-αM, α-α3) for 7 days after the 5 day differentiation was completed. Antibodies were free of azide, glycerol, and other preservatives.

### 4. Culture of Human and Mouse Fibroblasts

Human fibroblasts (MRC-5, ATCC, Manassas, VA) and mouse fibroblasts (NIH-3T3, ATCC) were cultured in SFM composed for human cells, with 1× concentrations of additives. 5 ng/ml TGFβ (Peprotech) was added to induce myofibroblast formation [74]. Cells were cultured at 10,000/cm², and TGFβ was refreshed in cultures weekly to maintain the myofibroblast phenotype, if necessary. Myofibroblasts were de-differentiated by addition of antibodies for 7 days.

5. mRNA Purification and RNAseq

mRNA was purified using the Trizol method [75]. RNA sequencing was performed by the University of Chicago Center for Research Informatics.

### 6. Flow Cytometry

Cultured myofibroblasts were lifted with ice cold trypsin-EDTA (Sigma), followed by mechanical agitation by a rubber policeman. Myofibroblasts were fixed and permeabilized using Cytofix/Cytoperm (BD biosciences, Franklin Lakes, NJ), and live-dead stained (live-dead aqua, ThermoFisher) per manufacturer's instructions. Antibodies used were α-collagen I (Biolegend, San Diego, CA), anti-α smooth muscle actin (αSMA) (R and D systems, Minneapolis, MN), α-ki-67 (BD biosciences), and α-talin2 (R and D systems). Compensation was performed via UltraComp beads (ThermoFisher) per the manufacturer's instructions.

### 7. Immunofluorescence

Human monocytes from 3 donors were culture in 8-well chamber slides (Millipore-Sigma) in SFM, and allowed to become myofibroblasts over 5 days. Myofibroblasts were then treated with antibodies against integrins α3 (α-α3), -M (α-αM), and αMβ2 (α-αMβ2) at 500 ng/ml for 1 week. After 1 week of de-differentiation, the slides were dried quickly using the airflow from a laminar flow hood, in order to preserve cellular morphology as accurately as possible. Cells were then fixed with ice cold 4% PFA, permeabilized with saponin (Sigma). Primary antibodies (α-talin2, novus) were added at 5 μg/ml overnight. Cells were gently washed 3 times in PBS, and were exposed to DAPI and F-actin-phalloidin-488 (ThermoFisher) for 1 min. Cells were mounted using water-based mowiol mounting media (Southern Biotech, Birmingham, AL) to preserve fluorescence. Slides were imaged immediately using a Confocal microscope (Olympus, Shinjuku City, Tokyo).

### 8. Legendplex Detection by ELISA

Supernatant from cultured human and mouse myofibroblasts were thawed at 4 C, and centrifuged at 4 C and 10,000×G to pellet cell debris. Supernatant was taken and added to 96 well round bottom plates, in duplicate. Legendplex (Biolegend) beads against general inflammation markers were added, according to the manufacturer's instructions. Sample readouts were normalized to each individual donor control.

### 9. Synthesis of Peptide Conjugated Antibody

Conjugation of decorin's collagen-binding peptide (CBP) (LRELHLNNNC SEQ ID NO:2) was performed as described previously [43]. Monoclonal antibodies, including mouse α-αM (clone ICRF44 for human experiments, clone M1/70 for mouse experiments, Biolegend), mouse anti-human/mouse α3 (α-α3, 3F9G4, Proteintech, Rosemont, IL), and mouse anti-human MAC1 (α-αMβ2, CBRM1/5, Biolegend) were incubated with 30 fold molar excess of sulfo-SMCC cross-linker (ThermoFisher) for 30 min at room temperature. The sulfo-SMCC-antibody was then mixed with 20, 30, and 40 fold molar excess CBP peptide (LRELHLNNNC SEQ ID NO:2) for 1 hr at room temperature, resulting in CBP-conjugated anti-integrin antibodies (CBP-α-αMβ2, CBP-α-αM, CBP-α-α3). The peptide was more than 95% purity, (Genscript, Piscataway, NJ).

### 10. Matrix-Assisted Laser Desorption/Ionization-Time-of-Flight (MALDI-TOF) Mass Spectroscopy

MALDI-TOF was performed as previously described [43]. Briefly, MALDI-TOF was performed on CBP-conjugated anti-integrin antibodies (CBP-α-αM, CBP-α-α3, CBP-α-αMβ2) using a UltrafleXtreme MALDI TOF/TOF instrument or a Bruker AutoFlex III Smartbeam MALDI TOF. Spectra were collected using Bruker flexControl software and processed with analysis software Bruker flexAnalysis or MATLAB (MathWorks). The matrix used was a saturated solution of α-cyano-4-hydroxycinnamic acid (Sigma-Aldrich) or sinapic acid (Sigma-Aldrich), was prepared in 50:50 (v/v) acetonitrile:(1% trifluoroacetic acid in water) as a solvent. The analyte in phosphate-buffered saline (PBS) (5 μl, 0.1 mg/ml) and the matrix solution (25 μl) were then mixed, and 1 μl of that mixture was deposited on the MTP 384 ground steel target plate. The drop was allowed to dry in a nitrogen gas flow, which resulted in the formation of uniform sample/matrix coprecipitate. All samples were analyzed using the high mass linear positive mode method with 5000 laser shots at a laser intensity of 75%. The measurements were externally calibrated at three points with a mix of carbonic anhydrase, phosphorylase B, and BSA.

### 11. In Vivo Biodistribution Study

An in vivo biodistribution study was conducted as previously described [43], with minor adjustments.

Fluorescently labeled Cy7-antibodies (CY7-α-αMβ2, CY7-α-αM, CY7-α-α3) were conjugated using sulfo-Cy7 N-hydroxysuccinimide ester (Lumiprobe) according to the manufacturer's instruction. Unreacted Cy7 was removed by dialysis against PBS.

To make fluorescently labeled Cy7-CBP-antibody, antibodies were incubated with eightfold molar excess of SM(PEG)₂₄ cross-linker (ThermoFisher) for 30 min at room temperature. Unreacted cross-linker was removed using a Zeba spin desalting column (Thermofisher, Waltham, MA), and then 30-fold molar excess of Cy7-labeled CBP ([Cy7]LRELHLNNNC[COOH—SEQ ID NO:2], Genscript, >95% purity) was added and reacted for 30 min at room temperature for conjugation to the thiol moiety on the C residue. Unreacted Cy7-CBP was removed by dialysis against PBS, resulting in antibodies labeled with CY7 and CBP: CY7-CBP-α-αMβ2, CY7-CBP-α-αM, and CY7-CBP-α-α3.

7 days following bleomycin insult or UUO surgery, 50 μg Cy7-CBP-antibody or Cy7-antibody were injected via tail vein. 48 hr later (in the case of lungs, FIG. 3) or 24 hours later (in the case of kidneys, FIG. 7), heart, lungs, spleen, kidneys, and liver were resected and imaged via IVIS (Xenogen) under the following conditions: f/stop, 2; optical filter excitation, 710 nm; emission, 780 nm; exposure time, 5 seconds; small binning.

### 12. Bleomycin Induced Pulmonary Fibrosis Model

Male and female mice were acquired at 8 weeks of age (Jackson laboratories, Bar Harbor, ME) with the intent to be used at 12 weeks of age. Due to delays regarding COVID19 lockdown and the allowed resumption of non-COVID19 research, the mice were 32 weeks old when the study began. Mouse lungs were instilled with 0.075 units bleomycin (75 μg, Fresenius Kabi, Switzerland) suspended in endotoxin-free PBS, as previously described [43]. First, mice were anesthetized via isoflurane inhalation (2%). Mice were then placed upright on an angled surface, their tongue pulled to the side, and a 200 μl narrow pipet was placed at the entrance of their throat. 50 μl of bleomycin/PBS was dispensed to the entrance of the throat, and mice were allowed to inhale. Administration to the lungs was confirmed by listening to the mouse's breathing for popping noises. Mice were then weighed and placed on a heating pad to recuperate.

Following bleomycin insult, mice were injected i.v. with 50 μg of antibody (α-αMβ2, α-αM, α-α3) or CBP-antibody (CBP-α-αMβ2, CBP-α-αM, CBP-α-α3) via tail-vein injection. The dose schedule was 7, 9, 11, 14, 16, and 18 days following bleomycin insult. Mice were euthanized at 21 days post insult via injecting of euthasol (Covetrus, Portland, ME) instead of CO₂ inhalation, which could damage the lungs.

### 13. Lung Resection and Fibrosis Scoring

Lungs were harvested, and perfused with 5 ml of PBS via cardiac puncture. After resection, the right and left lobes were separated. The left lobe was fixed in 4% paraformaldehyde overnight, mounted in paraffin, sectioned into 5 m slices, and stained using Masson's trichrome. Stained lungs were scanned at high resolution using a CRi Panoramic SCAN 40× Whole Slide Scanner (Perkin-Elmer, Waltham, MA), and were read for fibrosis using a modified Ashcroft method, as previously described [56]. Lungs were read unlabeled by a researcher uninvolved with animal treatment.

The right lobe of the lung was frozen, and dehydrated using a tissue lyophilizer (Labconco, Kansas City, MO), weighed, and was assessed for collagen content by hydroxyproline assay [76]. Briefly, dried lungs were digested in 6N HCl/PBS at 100 C for 24 hr. Supernatant from this digestion was added to 96 well plates and treated sequentially with chloramine-T solution and Ehrlich's solution at 65 C for 15 min to facilitate the color change reaction. Color was read at 561 nm. Quantification was provided by use of a hydroxyproline (Sigma) dilution series, which was transformed into a standard curve.

### 14. Kidney Unilateral Ureteral Obstruction (UUO) Fibrosis Model

UUO surgery was performed as previously described [77], with adjustments. Briefly, Mice were anesthetized via 2% isoflurane inhalation, and injected with meloxicam (1 mg/kg), buprenorphine (0.1 mg/kg) in a saline solution, subcutaneously. Briefly, mice were laid on their right side and an abdominal incision used to visualize the left ureter. The left ureter was ligated in the middle section of the ureter with two ties (2 mm apart) using 7-0 silk sutures. Peritoneum is then closed with 5-0 vicryl and skin is closed with 5-0 nylon.

1 week following UUO ligation, the mice were injected with 50 μg of antibody (α-TGFβ, α-α3, α-αMβ2) or CBP-antibody (CBP-α-TGFβ, CBP-α-α3, CBP-α-αMβ2) via tail-vein injection. 2 weeks post UUO, and 1 week post injection, mice were sacrificed via CO2 inhalation, and their kidneys harvested. At this point, the inventors checked to make sure that the UUO ligation was still in place, and in each case it was.

### 15. Assessment of Fibrosis in Kidneys

Right (healthy) and left (fibrotic) kidneys were placed in 4% PFA for 24 hours, mounted in paraffin, sectioned into 5 mm full kidney slices, and stained using immunohistochemistry (IHC) for collagen I (1:4000, polyclonal rabbit, lifespan biosciences, Seattle WA) via a Bond-Max autostaining system (Leica biosystems, Lincolnshire, IL). Stained kidneys were scanned at high resolution using a CRi Panoramic SCAN 40× Whole Slide Scanner (Perkin-Elmer).

Images were equalized in size, and converted to tif files using CaseViewer. Images were then imported into imageJ, scale set for conversion between microns and pixels, and deconvoluted with the “H DAB” deconvolution option. The resulting blue image was thresholded at 215 to see how many pixels were negative for collagen I, and the brown (IHC positive) image thresholded at 185 to see how many pixels were positive for collagen I. Machine-staining allowed these kidneys to be compared with high reproducibility.

### 16. Blood Analysis for Markers of Kidney Damage

At the time of euthanasia, blood was collected via submandibular bleed into protein low-bind tubes and allowed to coagulate for 2 hours on ice. Coagulated blood was then centrifuged at 10,000×G for 10 min, and serum collected. Serum was then diluted 4× in MilliQ water before being placed on deck on an Alfa Wassermann VetAxcel Blood Chemistry Analyzer. All tests requiring calibration were calibrated on the day of analysis and quality controls were run before analyzing samples. Serum tests were run according to kit instructions, and creatine kinase was normalized to calcium ion concentrations where indicated to account for sample hemolysis.

### 17. Statistical Analysis

Statistical analyses were performed using GraphPad Prism software, and P<0.05 was considered statistically significant. Either Student's t-test, 2-way ANOVA (with Fisher's LSD post test), or 1-way ANOVA with Welch's correction was used to compare groups.

### E. Abbreviations

alpha-smooth muscle actin (αSMA); CBP-functionalized blocking antibodies against αMβ2 (CBP-α-αMβ2) CBP-functionalized blocking antibodies against αM (CBP-α-αM); CBP-functionalized blocking antibodies against α3 (CBP-α-α3) collagen-binding peptide (CBP)(LRELHLNNNC—SEQ ID NO:2); fetal calf serum (FCS) Fibrillar adhesions (FBs); Fluorescently labeled Cy7-blocking antibodies against α3 (CY7-α-α3) Fluorescently labeled Cy7-blocking antibodies against αMβ2 (CY7-α-αMβ2) Fluorescently labeled Cy7-blocking antibodies against αM (CY7-α-αM) focal adhesions (FAs); Gi14 (antibody which stabilizes the association of integrin α2β1); Human fibroblasts (MRC-5) heterodimer αMβ2 (called MAC1) Idiopathic pulmonary fibrosis (IPF) IL-12 subunit p40 (IL12p40) Integrin-α3 (α3); Integrin-αM (αM); Integrin-β2 (β2) (CD18); macrophage-chemotactic protein-1 (MCP1) mouse fibroblasts (NIH-3T3); blocking antibodies against αMβ2 (α-αMβ2) blocking antibodies against αM (α-αM) blocking antibodies against α3 (α-α3) peripheral blood mononuclear cells (PBMC) phosphate buffered saline (PBS); serum-free media (SFM); Transforming growth factor β (TGFβ); Tumor necrosis factor α (TNFα) Unilateral ureteral obstruction (UUO)

### F. References

The following references and those listed throughout the patent application, to the extent that they provide exemplary procedural or other details supplementary to those set forth herein, are specifically incorporated herein by reference.
1. Wynn, T. A., Cellular and molecular mechanisms of fibrosis. J Pathol, 2008. 214(2): p. 199-210.
2. Wynn, T. A., Fibrotic disease and the T(H)1/T(H)2 paradigm. Nat Rev Immunol, 2004. 4(8): p. 583-94.
3. Spagnolo, P., Novel treatments for idiopathic pulmonary fibrosis. Am J Med, 2015.
4. Xaubet, A., A. Serrano-Mollar, and J. Ancochea, Pirfenidone for the treatment of idiopathic pulmonary fibrosis. Expert Opin Pharmacother, 2014. 15(2): p. 275-81.
5. Knuppel, L., et al., A Novel Antifibrotic Mechanism of Nintedanib and Pirfenidone. Inhibition of Collagen Fibril Assembly. Am J Respir Cell Mol Biol, 2017. 57(1): p. 77-90.
6. Raghu, G., et al., Long-term treatment with recombinant human pentraxin 2 protein inpatients with idiopathic pulmonary fibrosis: an open-label extension study. Lancet Respir Med, 2019. 7(8): p. 657-664.
7. Madsen, D. H., et al., M2-like macrophages are responsible for collagen degradation through a mannose receptor-mediated pathway. J Cell Biol, 2013. 202(6): p. 951-66.
8. Klingberg, F., B. Hinz, and E. S. White, The myofibroblast matrix: implications for tissue repair and fibrosis. J Pathol, 2013. 229(2): p. 298-309.
9. Hinz, B., Formation and function of the myofibroblast during tissue repair. J Invest Dermatol, 2007. 127(3): p. 526-37.
10. Liu, F., et al., Feedback amplification of fibrosis through matrix stiffening and COX-2 suppression. J Cell Biol, 2010. 190(4): p. 693-706.
11. Hinz, B. and G. Gabbiani, Fibrosis: recent advances in myofibroblast biology and new therapeutic perspectives. F1000 Biol Rep, 2010. 2: p. 78.
12. Hinz, B., Mechanical aspects of lung fibrosis: a spotlight on the myofibroblast. Proc Am Thorac Soc, 2012. 9(3): p. 137-47.
13. Tomasek, J. J., et al., Myofibroblasts and mechano-regulation of connective tissue remodelling. Nat Rev Mol Cell Biol, 2002. 3(5): p. 349-63.
14. Lagares, D., et al., Targeted apoptosis of myofibroblasts with the BH3 mimetic ABT-263 reverses established fibrosis. Sci Transl Med, 2017. 9(420).
15. Balestrini, J. L., et al., The mechanical memory of lung myofibroblasts. Integr Biol (Camb), 2012. 4(4): p. 410-21.
16. Abe, R., et al., Peripheral blood fibrocytes: differentiation pathway and migration to wound sites. J Immunol, 2001. 166(12): p. 7556-62.
17. Forbes, S. J., et al., A significant proportion of myofibroblasts are of bone marrow origin in human liver fibrosis. Gastroenterology, 2004. 126(4): p. 955-63.
18. Direkze, N. C., et al., Multiple organ engraftment by bone-marrow-derived myofibroblasts and fibroblasts in bone-marrow-transplanted mice. Stem Cells, 2003. 21(5): p. 514-20.
19. Hashimoto, N., et al., Bone marrow-derived progenitor cells in pulmonary fibrosis. J Clin Invest, 2004. 113(2): p. 243-52.
20. Nakashima, T., et al., Lung bone marrow-derived hematopoietic progenitor cells enhance pulmonary fibrosis. Am J Respir Crit Care Med, 2013. 188(8): p. 976-84.
21. Mori, L., et al., Fibrocytes contribute to the myofibroblast population in wounded skin and originate from the bone marrow. Exp Cell Res, 2005. 304(1): p. 81-90.
22. Murray, P. J., et al., Macrophage activation and polarization: nomenclature and experimental guidelines. Immunity, 2014. 41(1): p. 14-20.
23. Wynn, T. A. and T. R. Ramalingam, Mechanisms of fibrosis: therapeutic translation for fibrotic disease. Nat Med, 2012. 18(7): p. 1028-40.
24. Cox, D., M. Brennan, and N. Moran, Integrins as therapeutic targets: lessons and opportunities. Nat Rev Drug Discov, 2010. 9(10): p. 804-20.
25. Anthis, N. J., et al., Structural diversity in integrin talin interactions. Structure, 2010. 18(12): p. 1654-66.
26. Lawson, C., et al., FAK promotes recruitment of talin to nascent adhesions to control cell motility. J Cell Biol, 2012. 196(2): p. 223-32.
27. Anthis, N. J., et al., The structure of an integrin talin complex reveals the basis of inside-out signal transduction. EMBO J, 2009. 28(22): p. 3623-32.
28. Roca-Cusachs, P., et al., Clustering of alpha(5)beta(1) integrins determines adhesion strength whereas alpha(v)beta(3) and talin enable mechanotransduction. Proc Natl Acad Sci USA, 2009. 106(38): p. 16245-50.
29. Dugina, V., et al., Focal adhesion features during myofibroblastic differentiation are controlled by intracellular and extracellular factors. J Cell Sci, 2001. 114(Pt 18): p. 3285-96.
30. Hinz, B. and G. Gabbiani, Cell-matrix and cell-cell contacts of myofibroblasts: role in connective tissue remodeling. Thromb Haemost, 2003. 90(6): p. 993-1002.
31. Hoffman, B. D., C. Grashoff, and M. A. Schwartz, Dynamic molecular processes mediate cellular mechanotransduction. Nature, 2011. 475(7356): p. 316-23.
32. Geiger, B., J. P. Spatz, and A. D. Bershadsky, Environmental sensing through focal adhesions. Nat Rev Mol Cell Biol, 2009. 10(1): p. 21-33.
33. De, R., A. Zemel, and S. A. Safran, Theoretical concepts and models of cellular mechanosensing. Methods Cell Biol, 2010. 98: p. 143-75.
34. Parsons, J. T., A. R. Horwitz, and M. A. Schwartz, Cell adhesion: integrating cytoskeletal dynamics and cellular tension. Nat Rev Mol Cell Biol, 2010. 11(9): p. 633-43.
35. Katsumi, A., et al., Integrins in mechanotransduction. J Biol Chem, 2004. 279(13): p. 12001-4.
36. Praekelt, U., et al., New isoform-specific monoclonal antibodies reveal different sub-cellular localisations for talin1 and talin2. Eur J Cell Biol, 2012. 91(3): p. 180-91.
37. Hyun, Y. M., C. T. Lefort, and M. Kim, Leukocyte integrins and their ligand interactions. Immunol Res, 2009. 45(2-3): p. 195-208.
38. Podolnikova, N. P., et al., Ligand recognition specificity of leukocyte integrin alphaMbeta2 (Mac-1, CD11b/CD18) and its functional consequences. Biochemistry, 2015. 54(6): p. 1408-20.
39. Satoh, T., et al., Identification of an atypical monocyte and committed progenitor involved infibrosis. Nature, 2017. 541(7635): p. 96-101.
40. Lomakina, E., et al., Activation of human neutrophil Mac-1 by anion substitution. Blood Cells Mol Dis, 2009. 42(3): p. 177-84.
41. Maiguel, D., et al., Small molecule-mediated activation of the integrin CD11b/CD18 reduces inflammatory disease. Sci Signal, 2011. 4(189): p. ra57.
42. Nishiuchi, R., et al., Characterization of the ligand-binding specificities of integrin alpha3beta1 and alpha6beta1 using a panel of purified laminin isoforms containing distinct alpha chains. J Biochem, 2003. 134(4): p. 497-504.
43. Katsumata, K., et al., Targeting inflammatory sites through collagen affinity enhances the therapeutic efficacy of anti-inflammatory antibodies. Sci Adv, 2019. 5(11): p. eaay1971.
44. Michael J V White, Melis Ozkan, Jorge Emiliano Gomez-Medellin, Jeffrey A Hubbell Myofibroblast differentiation is governed by adhesion mechanics, and inhibition of the stress sensor Talin2 reverses lung fibrosis. 2021. DOI: https://doi.org/10.1101/202106.07.447403.
45. Walzog, B., et al., Beta2 integrins (CD11/CD18) promote apoptosis of human neutrophils. FASEB J, 1997. 11(13): p. 1177-86.
46. Camper, L., D. Heinegard, and E. Lundgren-Akerlund, Integrin alpha2beta1 is a receptor for the cartilage matrix protein chondroadherin. J Cell Biol, 1997. 138(5): p. 1159-67.
47. Beller, D. I., T. A. Springer, and R. D. Schreiber, Anti-Mac-1 selectively inhibits the mouse and human type three complement receptor. J Exp Med, 1982. 156(4): p. 1000-9.
48. Oxvig, C., C. Lu, and T. A. Springer, Conformational changes in tertiary structure near the ligand binding site of an integrin I domain. Proc Natl Acad Sci USA, 1999. 96(5): p. 2215-20.
49. Gasse, P., et al., IL-1 and IL-23 mediate early IL-17A production in pulmonary inflammation leading to late fibrosis. PLoS One, 2011. 6(8): p. e23185.
50. Yogo, Y., et al., Macrophage derived chemokine (CCL22), thymus and activation-regulated chemokine (CCL17), and CCR4 in idiopathic pulmonary fibrosis. Respir Res, 2009. 10: p. 80.
51. Le, T. T., et al., Blockade of IL-6 Trans signaling attenuates pulmonary fibrosis. J Immunol, 2014. 193(7): p. 3755-68.
52. Belperio, J. A., et al., The role of the Th2 CC chemokine ligand CCL17 in pulmonary fibrosis. J Immunol, 2004. 173(7): p. 4692-8.
53. Huaux, F., et al., A profibrotic function of IL-12p40 in experimental pulmonary fibrosis. J Immunol, 2002. 169(5): p. 2653-61.
54. Sahin, H. and H. E. Wasmuth, Chemokines in tissue fibrosis. Biochim Biophys Acta, 2013. 1832(7): p. 1041-8.
55. Oikonomou, N., et al., Soluble TNF mediates the transition from pulmonary inflammation to fibrosis. PLoS One, 2006. 1: p. e108.
56. Hubner, R. H., et al., Standardized quantification of pulmonary fibrosis in histological samples. Biotechniques, 2008. 44(4): p. 507-11, 514-7.
57. Neumark, N., et al., The Idiopathic Pulmonary Fibrosis Cell Atlas. Am J Physiol Lung Cell Mol Physiol, 2020. 319(6): p. L887-L893.
58. Rabb, H., et al., Role of CD11a and CD11b in ischemic acute renalfailure in rats. Am J Physiol, 1994. 267(6 Pt 2): p. F1052-8.
59. Dehnadi, A., et al., Prophylactic orthosteric inhibition of leukocyte integrin CD11b/CD18 prevents long-term fibrotic kidney failure in cynomolgus monkeys. Nat Commun, 2017. 8: p. 13899.
60. Hinz, B., Masters and servants of the force: the role of matrix adhesions in myofibroblast force perception and transmission. Eur J Cell Biol, 2006. 85(3-4): p. 175-81.
61. Tang, T., et al., A role for Mac-1 (CD11b/CD18) in immune complex-stimulated neutrophil function in vivo: Mac-1 deficiency abrogates sustained Fcgamma receptor-dependent neutrophil adhesion and complement-dependent proteinuria in acute glomerulonephritis. J Exp Med, 1997. 186(11): p. 1853-63.
62. Lim, J., et al., An essential role for talin during alpha(M)beta(2)-mediated phagocytosis. Mol Biol Cell, 2007. 18(3): p. 976-85.
63. Zhang, X., et al., Talin depletion reveals independence of initial cell spreading from integrin activation and traction. Nat Cell Biol, 2008. 10(9): p. 1062-8.
64. Austen, K., et al., Extracellular rigidity sensing by talin isoform-specific mechanical linkages. Nat Cell Biol, 2015. 17(12): p. 1597-606.
65. Petrich, B. G., et al., The antithrombotic potential of selective blockade of talin-dependent integrin alpha IIb beta 3 (platelet GPIIb-IIIa) activation. J Clin Invest, 2007. 117(8): p. 2250-9.
66. Li, Y. F., et al., The cytosolic protein talin induces an intermediate affinity integrin alphaLbeta2. J Biol Chem, 2007. 282(33): p. 24310-9.
67. Bruinsma, R., Theory of force regulation by nascent adhesion sites. Biophys J, 2005. 89(1): p. 87-94.
68. Chan, C. E. and D. J. Odde, Traction dynamics of filopodia on compliant substrates. Science, 2008. 322(5908): p. 1687-91.
69. Li, Y., P. Bhimalapuram, and A. R. Dinner, Model for how retrograde actin flow regulates adhesion traction stresses. J Phys Condens Matter, 2010. 22(19): p. 194113.
70. Furuichi, K., et al., CCR2 signaling contributes to ischemia-reperfusion injury in kidney. J Am Soc Nephrol, 2003. 14(10): p. 2503-15.
71. Gallucci, R. M., E. G. Lee, and J. J. Tomasek, IL-6 modulates alpha-smooth muscle actin expression in dermal fibroblasts from IL-6-deficient mice. J Invest Dermatol, 2006. 126(3): p. 561-8.
72. Pilling, D., V. Vakil, and R. H. Gomer, Improved serum-free culture conditions for the differentiation of human and murine fibrocytes. Journal of immunological methods, 2009. 351(1-2): p. 62-70.
73. Crawford, J. R., D. Pilling, and R. H. Gomer, Improved serum-free culture conditions for spleen-derived murine fibrocytes. J Immunol Methods, 2010. 363(1): p. 9-20.
74. Hinz, B., et al., Myofibroblast development is characterized by specific cell-cell adherens junctions. Mol Biol Cell, 2004. 15(9): p. 4310-20.
75. Rio, D. C., et al., Purification of RNA using TRIzol (TRI reagent). Cold Spring Harb Protoc, 2010. 2010(6): p. pdb prot5439.
76. Bergheim, I., et al., Critical role of plasminogen activator inhibitor-1 in cholestatic liver injury and fibrosis. J Pharmacol Exp Ther, 2006. 316(2): p. 592-600.
77. Cochrane, A. L., et al., Renal structural and functional repair in a mouse model of reversal of ureteral obstruction. J Am Soc Nephrol, 2005. 16(12): p. 3623-30.
78. Subramanian, A., et al., Gene set enrichment analysis: a knowledge-based approach for interpreting genome-wide expression profiles. Proc Natl Acad Sci USA, 2005. 102(43): p. 15545-50.

### G. Tables

TABLE 1
Changes in mRNA expression from culturing monocytes on soft, anti-fibrotic
(1 kPa) and stiff, pro-fibrotic (12 kPa) surfaces, assessed by RNAseq. Enrichment
score is calculated based on the maximum deviation from 0 in the pathway
analysis [78]. In this analysis, 5.18 is the highest possible score.

| | Functional | | Investigate d | | Investigated | |
|---|---|---|---|---|---|---|
| Upregulated | annotation | Enrichment | protein | Fold | protein | Statistical |
| pathways | clustering | score | targets | chang e | targets | significanc e |
| Chemokine | Complement | 5.18 | ITGAM | 1.797671 | ITGB2 | 9.15E−04 |
| signaling | activation | | | | | |
| Leukocyte | Chemokine | 5.18 | ITGA3 | 3.536679 | ITGB1 | 2.59E−02 |
| extravasatio n | signaling | | | | | |
| signaling | | | | | | |
| IL-6 signaling | Phagocytosis | 5.18 | ITGA7 | 4.487744 | ITGAX | 4.75E−05 |
| | recognition | | | | | |
| Paxillin | positive | 5.18 | | | | |
| signaling | regulation of | | | | | |
| | inflammator y | | | | | |
| | response | | | | | |
| Integrin | monocyte | 5.18 | | | | |
| signaling | chemotaxis | | | | | |
| IL-13 | receptor- | 5.18 | | | | |
| signaling | mediated | | | | | |
| | endocytosis | | | | | |

TABLE 2
IC50 for α-α3, α-αM, and α-αMβ2 for
human monocyte-myofibroblast differentiation

| Treatment - Antibody | IC50 (ng/ml) | p value |
|---|---|---|
| α-αM (clone ICRF44) | 59.71 | 0.0111 |
| α-αMβ2 (MAC1, clone CBRM1/5) | 63.33 | 0.0019 |
| α-α3 (clone 3F9G4) | 78.36 | 0.0001 |

### Example 2: Bleomycin-Induced Pulmonary Fibrosis Model

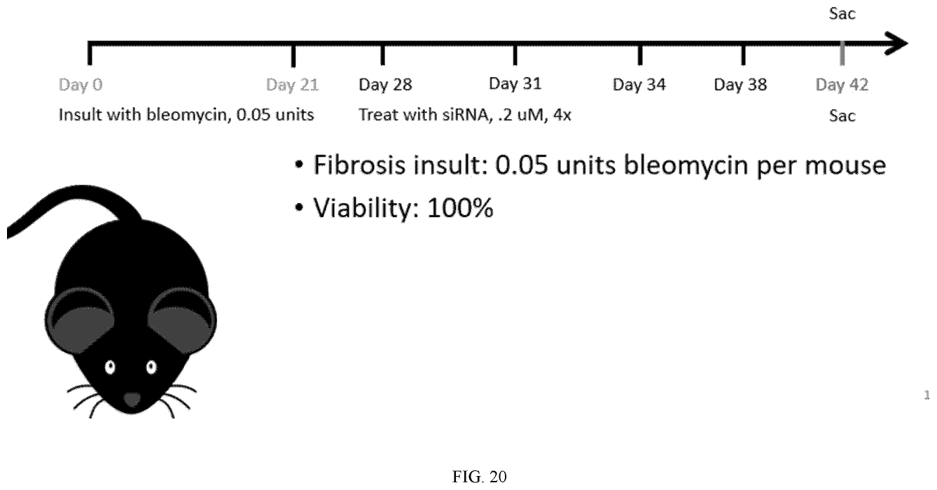

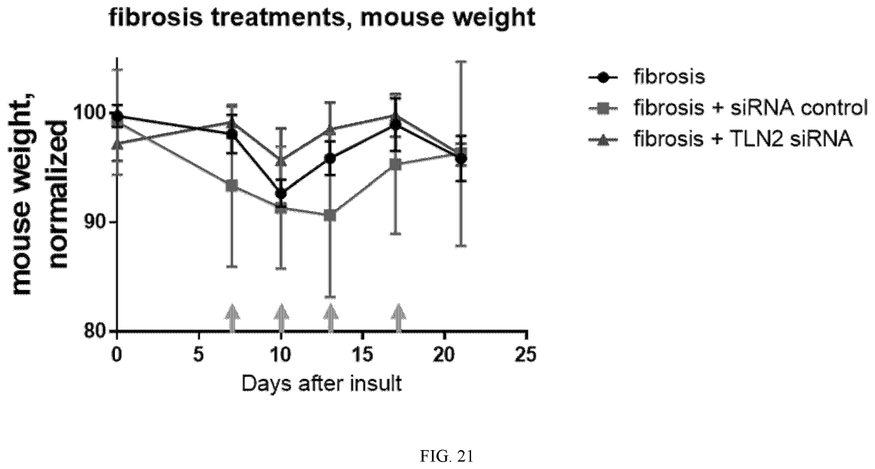

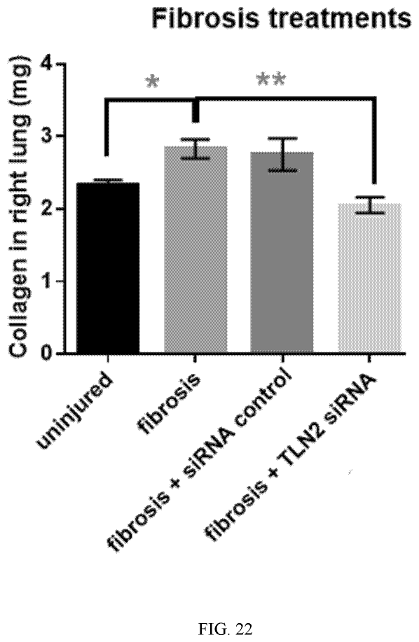

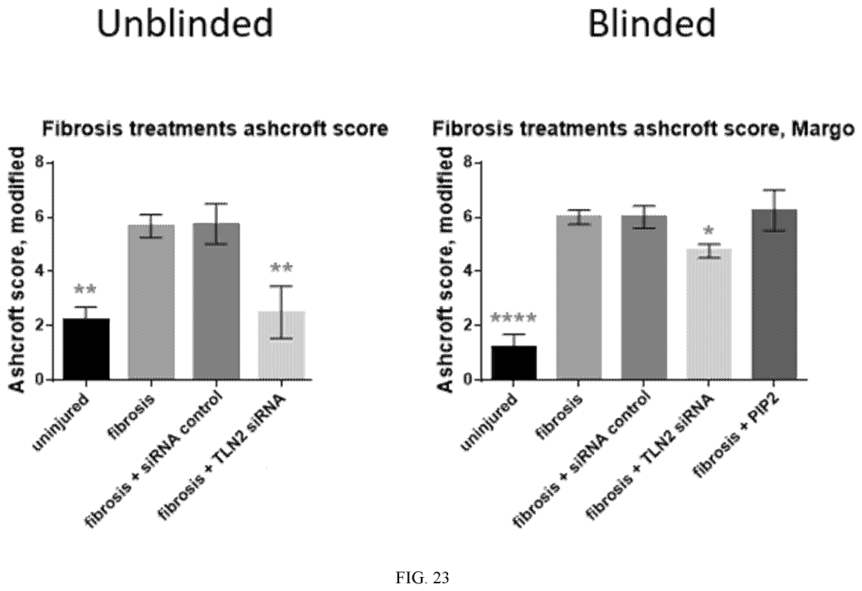

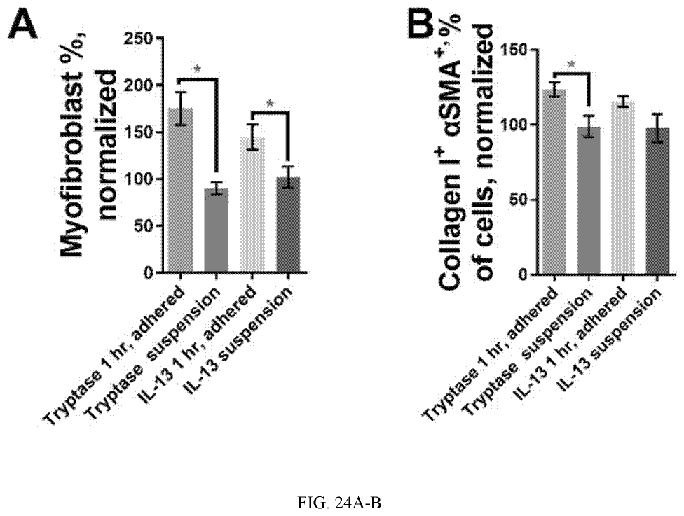

To determine whether targeting Talin2 can be used as a therapeutic for treating fibrosis, a bleomycin-induced pulmonary fibrosis model was studied. FIG. 20 shows a schematic of the dosing schedule for the bleomycin-induced pulmonary fibrosis experiment. Mice were insulted twice with bleomycin instilled into lungs, at day −21 and at day 0. 50 ml of 0.2 mM talin2 siRNA was administered to mouse lungs 7, 10, 13, and 17 days after insult by bleomycin. The mice were then harvested and analyzed on day 42. Talin2 siRNA was found to reverse the fibrotic damage from bleomycin insult to mouse lungs (FIG. 21, n=4). FIG. 22 shows the collagen content from the right, multi-lobed lung assessed by hydroxyproline assay. N=4, statistics are student's t-test. FIG. 23 shows an unblinded and blinded Ashcroft scoring of lung images. n=4. statistics are student's t-test. These studies demonstrate that targeting of talin2 can be used to treat pulmonary fibrosis in a bleomycin-induced pulmonary fibrosis model.

### Example 3: Myofibroblast Differentiation is Governed by Adhesion Mechanics, and Inhibition of Talin2 Reverses Lung and Kidney Fibrosis

The reference numbers cited in this example find their reference key at the end of this example. These reference numbers and reference key are specifically applicable to this example.

Fibrosis is involved in 45% of deaths in the United States, and no treatment exists to reverse progression of the disease. In order to find novel targets for fibrosis therapeutics, a model for the differentiation of monocytes to myofibroblasts was developed that allowed screening for proteins involved in myofibroblast differentiation. Inhibition of a novel protein target generated by the model, talin2, reduced myofibroblast morphology, α-smooth muscle actin content, collagen I content, and lowered the pro-fibrotic secretome of myofibroblasts. The results show that knockdown of talin2 de-differentiated myofibroblasts, talin2 knockdown reversed bleomycin-induced lung fibrosis in mice, and Tln2−/− mice were resistant to unilateral ureteral obstruction-induced kidney fibrosis and were resistant to bleomycin-induced lung fibrosis. The results showed that Talin2 inhibition is a potential treatment for reversing lung and kidney fibroses.

### A. Introduction

Fibrosing diseases—including pulmonary fibrosis, congestive heart failure, liver cirrhosis, and end-stage kidney disease—are involved in 45% of deaths in the United States [1, 2]. There are few FDA approved treatments for fibrosis [2, 3]. Currently approved FDA treatments (pirfenidone and nintedanib) slow, but do not reverse, the progression of fibrosis [4]. Further, the mechanisms of action of pirfenidone and nintedanib are poorly understood [5]. To date, only one treatment (recombinant pentraxin-2, PRM-151) has shown even a modest ability to reverse fibrosis in some patients [6].

The ultimate goal of any treatment is to reverse fibrosis [1]. Interrupting collagen deposition destabilizes scar tissue and is a necessary prerequisite for reversing fibrosis [1]. Another prerequisite for reversing fibrosis is removing deposited ECM while regenerating tissue, of which monocyte-derived cells are capable [7].

Herein the inventors show that monocytes and fibroblasts only differentiate into myofibroblasts when adhered to a surface of sufficient stiffness. The inventors were motivated to target talin2 by an RNAseq (mRNA sequencing) study comparing gene expression of myofibroblasts cultured on pro-fibrotic, sufficiently stiff surfaces versus culture on anti-fibrotic, insufficiently stiff surfaces. Knockdown of talin2 interrupts the ability of monocytes to sense stiffness, prevent monocyte-myofibroblast differentiation, and de-differentiates myofibroblasts. Furthermore, the inventors show that knockdown of talin2 can have therapeutic utility in treating pulmonary fibrosis.

### B. Results

### 1. Myofibroblast Differentiation was Governed by Adhesion and Substrate Stiffness.

The inventors began the studies described in this Example by investigating the features of cell adhesion that lead to efficient differentiation of monocytes into myofibroblasts. The inventors sought to identify a key pathway that could be inhibited to prevent differentiation. The results of this analysis led to the identification of a key role of talin2, knockdown of which both prevented differentiation of monocytes into myofibroblasts and moreover dedifferentiated existing myofibroblasts. Subsequently, the effects of talin2 knockdown in a murine model of pulmonary fibrosis was investigated, these results confirmed a key role of talin2.

The results demonstrated that monocyte differentiation into myofibroblasts was governed by the cell adherent state, including the elastic modulus of the adhesion substrate. First, it was determined that when exposed to pro-fibrotic factors (e.g., tryptase, IL-13) for 1 hr, human monocytes in suspension that are later cultured adherently do not differentiate efficiently into myofibroblasts (as determined by morphological demonstration of a clear spindle shape), compared to monocytes exposed to the same pro-fibrotic factors for the same amount of time while adhered (FIG. 24A). Adhered monocytes also increased their double-positive αSMA and collagen I content (FIG. 24B), when compared to suspended-then-adhered monocytes, as determined by flow cytometry.

Second, human monocytes were found to be unable to differentiate into myofibroblasts on surfaces softer than 1 kPa, even when adherent; monocyte-myofibroblast differentiation can occur and be potentiated at 12 kPa (FIG. 24C) in the presence of a pro-fibrotic factor. For reference, Table 3 (data from [45], [46]) shows the stiffnesses of various human tissues. Culture on surfaces that had been pre-coated with the ECM protein fibronectin supported myofibroblast differentiation even without pre-treatment with a pro-fibrotic factor, yet in a manner that is dependent on the substrate stiffness, with monocytes cultured on 1, 12, and functionally infinite kPa fibronectin-coated surfaces showing increasing amounts of myofibroblast differentiation (FIG. 24D), including increasing percentages of cells double-positive for αSMA and collagen I (FIG. 24E). Cultured fibroblasts also increased the percentage of αSMA- and collagen I-double-positive cells on higher surface stiffness fibronectin-coated substrates, both in the absence and presence of pro-fibrotic factors (FIG. 24F).

### 2. Talin2 is Upregulated in Myofibroblasts Cultured on Stiff Surfaces.

Using these results (e.g., FIG. 24C-E) as a guide, human monocytes from three donors were then cultured on 1 and 12 kPa fibronectin-coated surfaces, total mRNA was subsequently isolated from the population, and an RNAseq investigation was performed (see Tables 4 and 5). This RNAseq analysis revealed genes that were differentially expressed between monocytes cultured at 1 and 12 kPa, yielding both down-regulated and up-regulated individual genes and pathways. Individual upregulated genes included collagens (e.g., collagen XXII upregulated 22-fold) and chemotactic factors (CCL22 upregulated 13-fold). However, analysis of the upregulated pathway allowed for a more inclusive and complete picture of the changes induced by culturing monocytes on stiffer surfaces under pro-fibrotic conditions. Specifically, upregulated pathways related to cell adhesion included the paxillin and integrin pathways. Among the individual genes that were downregulated at 1 kPa (e.g., not supporting myofibroblast differentiation) relative to 12 kPa (e.g., supporting), the stress sensor talin2 was identified. Talin2 was reduced by 3-fold on the surface that did not allow monocyte-to-myofibroblast differentiation. Based on this observation, the remainder of this Example is focused on this intracellular modulator of adhesion mechanics, the tension-sensing protein talin2. The potential of integrins to modulate myofibroblast differentiation in a companion study (e.g., [47], Example 1).

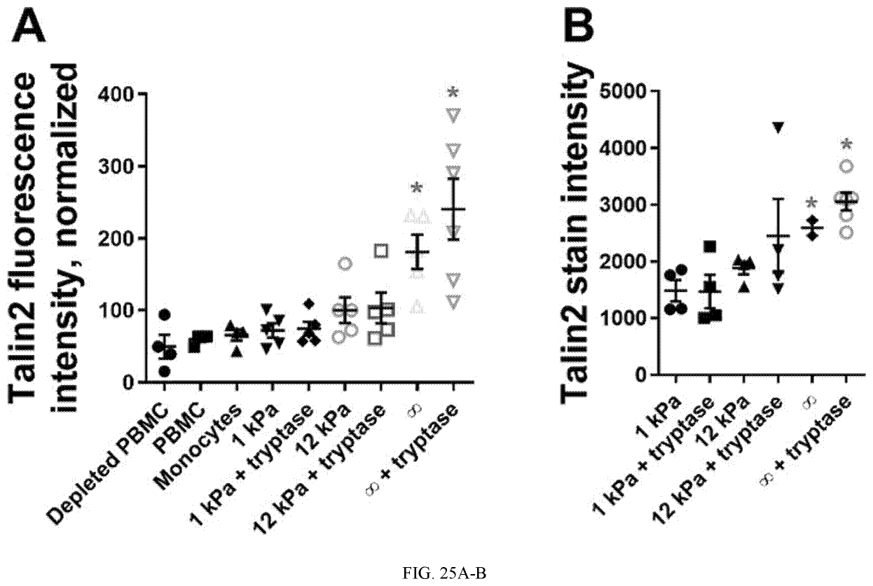

To confirm that the measured mRNA reduction in the sequence for talin2 at low stiffness corresponded to a drop in the protein talin2, monocytes and fibroblasts were cultured on surfaces of increasing stiffness in the presence and absence of a pro-fibrotic signal. Expanding on the staining for collagen I and αSMA in FIG. 24E-F, talin2 was low at 1 kPa and increased in both human monocytes (FIG. 25A) and human fibroblasts (FIG. 25B) on stiffer surfaces in more pro-fibrotic environments.

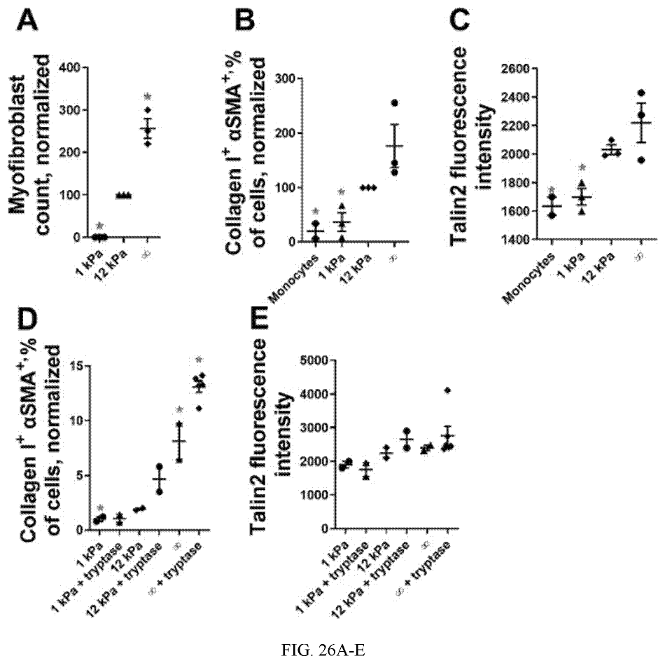

To determine if mouse cells also increase talin2 in more pro-fibrotic environments on stiffer surfaces, monocytes purified from mouse spleens and mouse fibroblasts were cultured under conditions similar to those used in the study of human cells reported in FIGS. 24 and 25. Mouse monocytes differentiated into morphologically spindle-shaped myofibroblasts with increasing surface stiffness (FIG. 26A), increasing positivity for αSMA and collagen I staining (FIG. 26B), and increasing talin2 staining (FIG. 26C). Mouse fibroblasts also increased in αSMA and collagen I staining (FIG. 26D) and talin2 staining (FIG. 26E) on stiffer surfaces in presence of tryptase.

### 3. Inhibition of Talin2 Reverses Myofibroblast Differentiation and Existing Fibrosis.

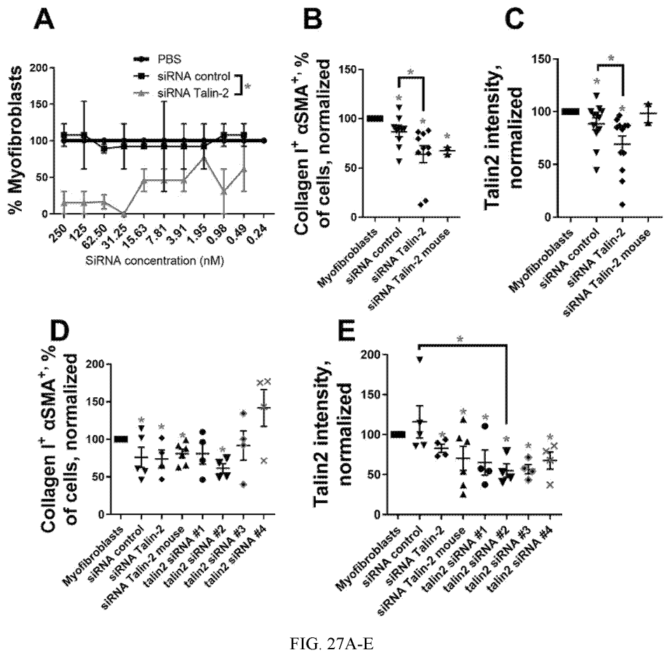

To determine if inhibition of talin2 expression could de-differentiate myofibroblasts, human and mouse monocytes were allowed to become myofibroblasts, and those myofibroblasts were treated with a mixture of 4 non-targeting silencing RNAs (siRNA), 4 human talin2 siRNAs, and 4 mouse talin2 siRNAs, respectively. The mouse and human talin2 siRNA mixtures share one sequence in overlap. To establish a dose range, human monocyte-derived myofibroblasts were treated with the talin2 siRNA mixture, yielding an IC50 of 15 nM (FIG. 27A) for inhibition of myofibroblast morphology. Treatment with 50 nM of talin2 siRNA also reduced the percentage of αSMA and collagen I positive human monocytes (FIG. 27B), more than the control siRNA mixture. Only the human talin2 siRNA significantly reduced the amount of talin2 (FIG. 27C) for human monocyte-derived myofibroblasts.

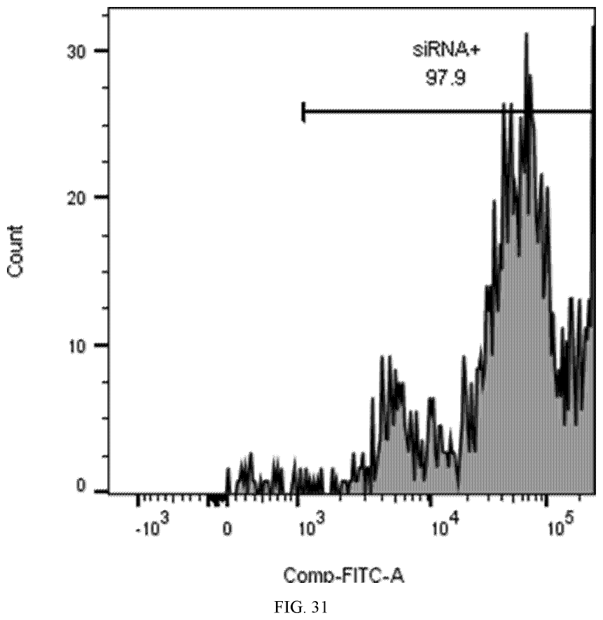

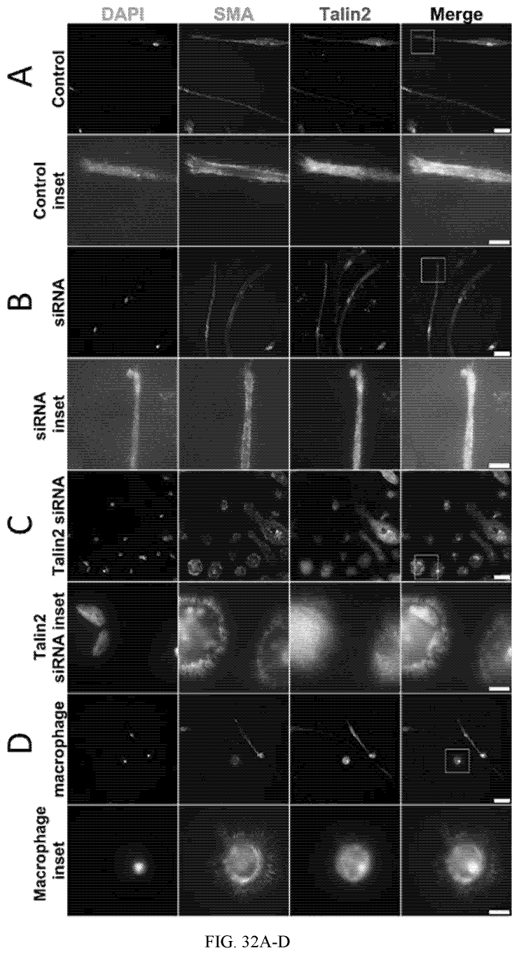

Treatment with fluorescently labeled siRNA (siRNA-AF-488) indicated that 50 nM siRNA was capable of entering human monocyte-derived myofibroblasts without the use of transfection reagents (FIG. 31). The mixture of talin2 siRNA reduced not only the spindle-shaped morphology within the monocyte-derived myofibroblast population, but also the presence of FAs at the cell periphery, FBs within cells, and the localization of talin2 to the periphery of the cell (FIG. 32).

In order to simplify treatment in anticipation of in vivo testing, mouse myofibroblasts were treated with individual siRNAs against mouse talin2, in addition to the same non-targeting, human talin2, and mouse talin2 siRNA mixtures as the human monocytes were treated with.

While each of the siRNA mixtures significantly decreased the number of αSMA and collagen I double-positive cells (FIG. 27D), only talin2 siRNA #2 (sequence: 5′ CUGGAAAAUUCAGUGAUGA 3′ [SEQ ID NO:26) and antisense 5′ UCAUCACUGAAUUUUCCAG 3′ [SEQ ID NO:37) inhibited myofibroblast differentiation by itself. All mixtures and individual siRNAs decreased talin2 concentration within the cells (FIG. 27E).

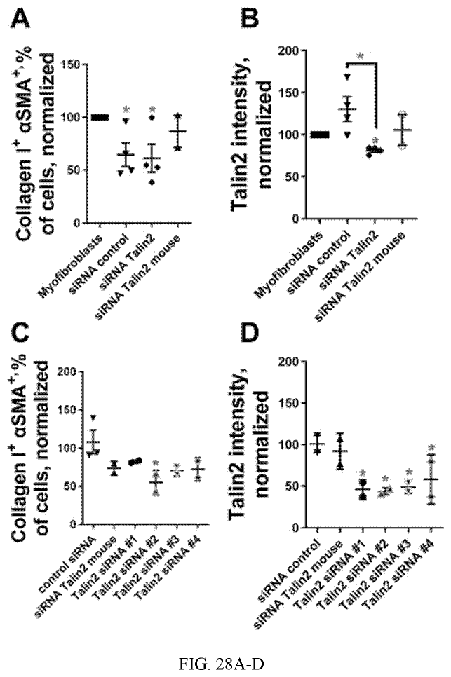

To determine if silencing talin2 can reduce fibroblast-myofibroblast differentiation, siRNAs were added individually and in mixtures at 50 nM to human and mouse fibroblast cultures. The mixture of human talin2 siRNA reduced both αSMA and collagen I double-positive (FIG. 28A) and talin2 positive (FIG. 28B) myofibroblasts. Mouse siRNA #2 most reduced both αSMA and collagen I double-positive (FIG. 28C) and talin2 positive (FIG. 28D) myofibroblasts in mouse cell cultures.

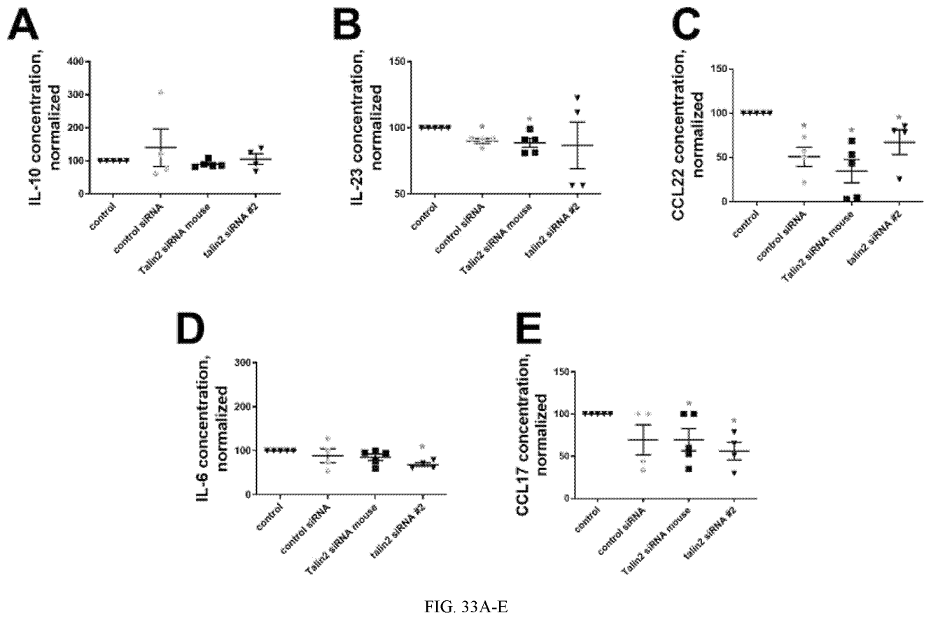

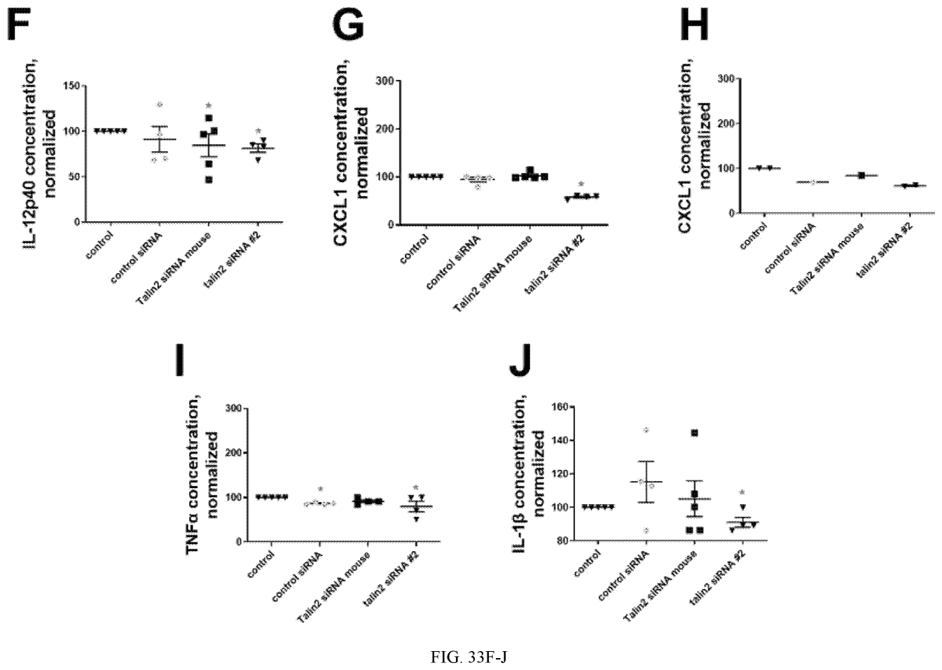

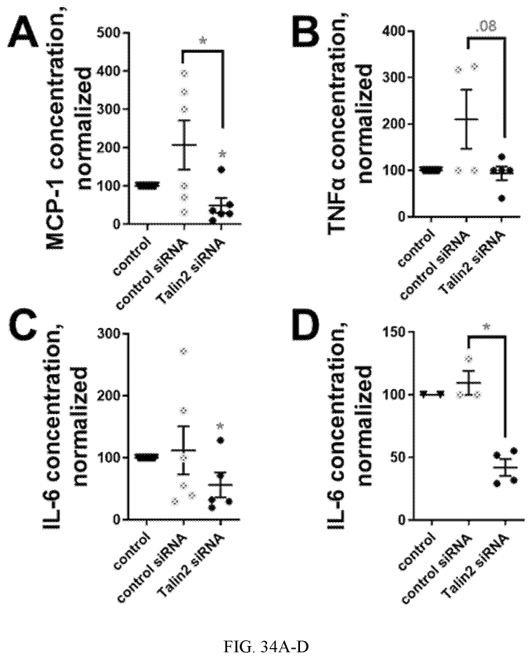

To determine if reductions in myofibroblast's spindle-shaped morphology, αSMA and collagen I content, and talin2 content correlated with a reduction in secreted pro-fibrotic factors, conditioned media from human and mouse monocyte-derived and fibroblast-derived myofibroblasts were assayed by ELISA. Treatment with talin2 siRNA did not significantly affect the amount of secreted anti-fibrotic IL-10 (FIG. 33A) [48], but did reduce the amount of pro-fibrotic cytokines including IL-23 (FIG. 33B) [49], CCL22 (FIG. 33C) [50], IL-6 (FIG. 33D) [51], CCL17 (FIG. 33E) [50, 52], IL-12 subunit p40 (FIG. 33F) [53], CXCL1 (FIG. 33G-H) [54], TNF-α (FIG. 331) [55], and IL-1β (FIG. 33J) [56]. While TGF-β was below the detection limit for this assay, IL-6 is sometimes sufficient to induce myofibroblast differentiation [57]. While the secretome for human cells was more frequently below the detection limit for this assay, similar results were seen, with reductions in pro-fibrotic TNF-α, MCP-1 [58], and IL-6 (FIG. 34). This indicates that disruption of talin2 was not simply changing myofibroblast morphology, or talin2 localization, but was also dramatically altering the overall secretome of treated cells.

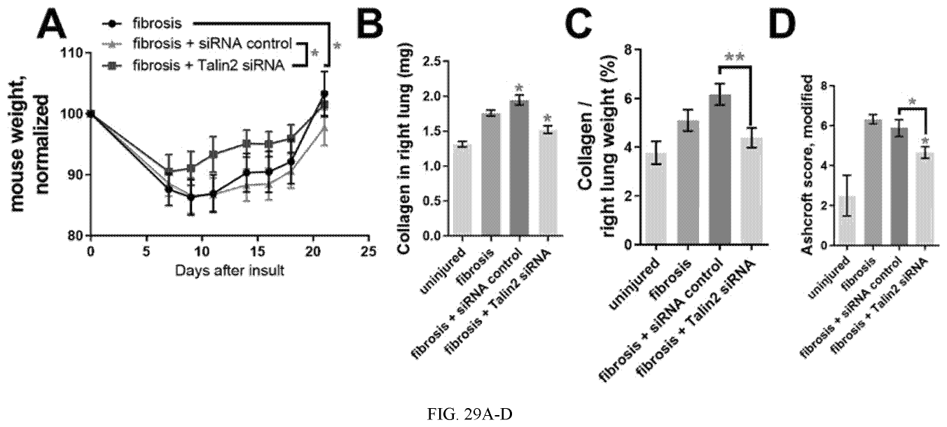

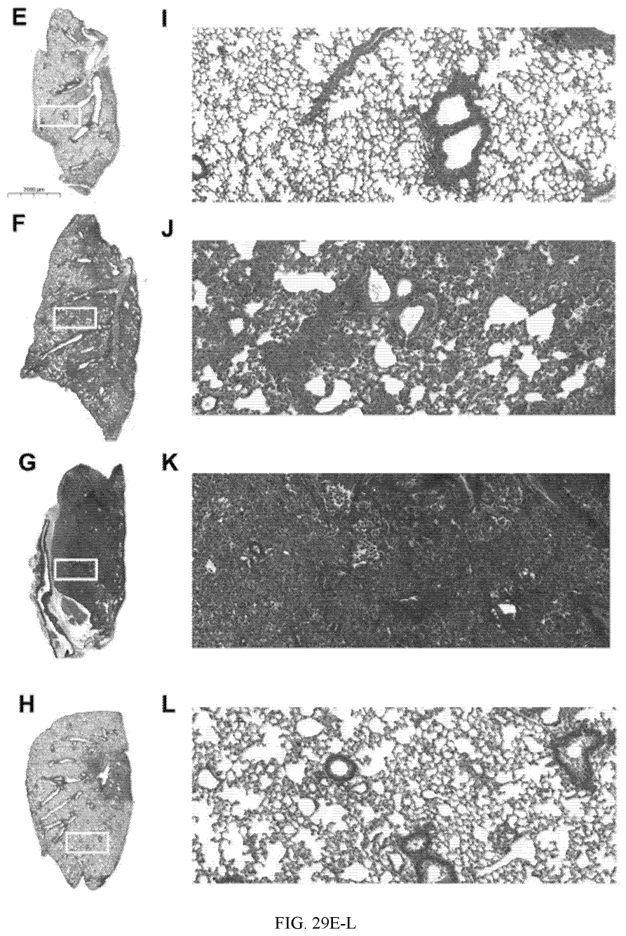

To determine if treatment with talin2 siRNA could rescue the damage from lung fibrosis in a treatment (not prophylactic) model, the lungs of male and female mice were insulted with bleomycin. The mice were treated via lung instillation with 200 nM of talin2 siRNA #2 at 7, 9, 11, 14, 16, and 18 days post bleomycin insult, and mice were euthanized on day 21 post insult. While no treatment significantly altered mouse weight at day 21, treatment with talin2 siRNA showed a lesser transient weight decrease than did the controls (FIG. 29A). Talin2 siRNA treatment also reduced the amount of collagen in the lungs as measured by a hydroxyproline assay (FIG. 29B-C). Blinded Ashcroft scoring [59] of the Masson's trichrome-stained lung sections, performed by another researcher, confirm that talin2 siRNA treatment improved the histological readout of lung fibrosis (FIG. 29D). The individual talin2 siRNA treatment rescued lung fibrosis compared to non-targeting siRNA and untreated fibrotic lungs (FIG. 29E-L). Thus, talin2 siRNA treatment rescued lung fibrosis in mice in both quantitative (hydroxyproline) and qualitative (Ashcroft scoring) measures, when treatment was provided 7 days after the inflammatory insult to the lung.

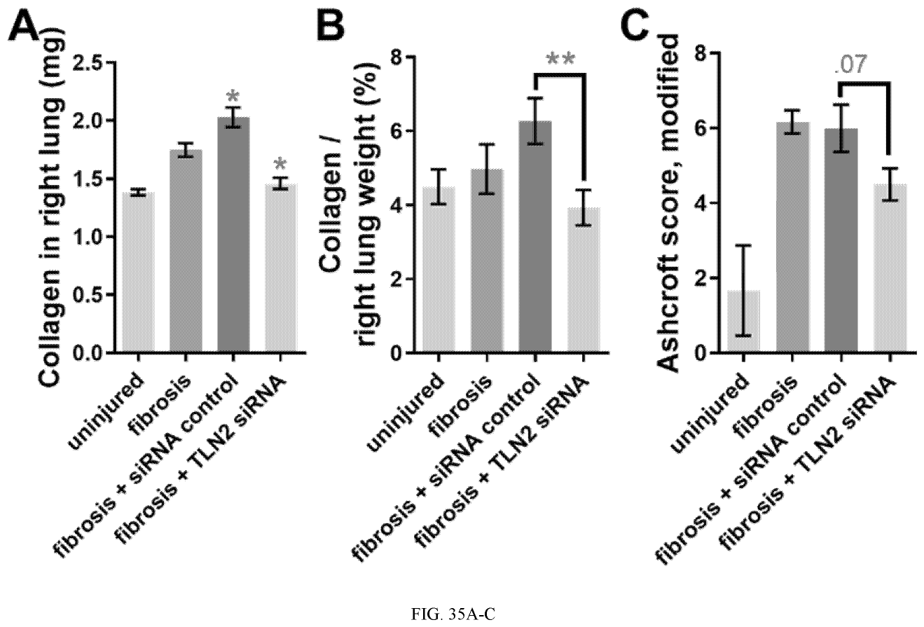

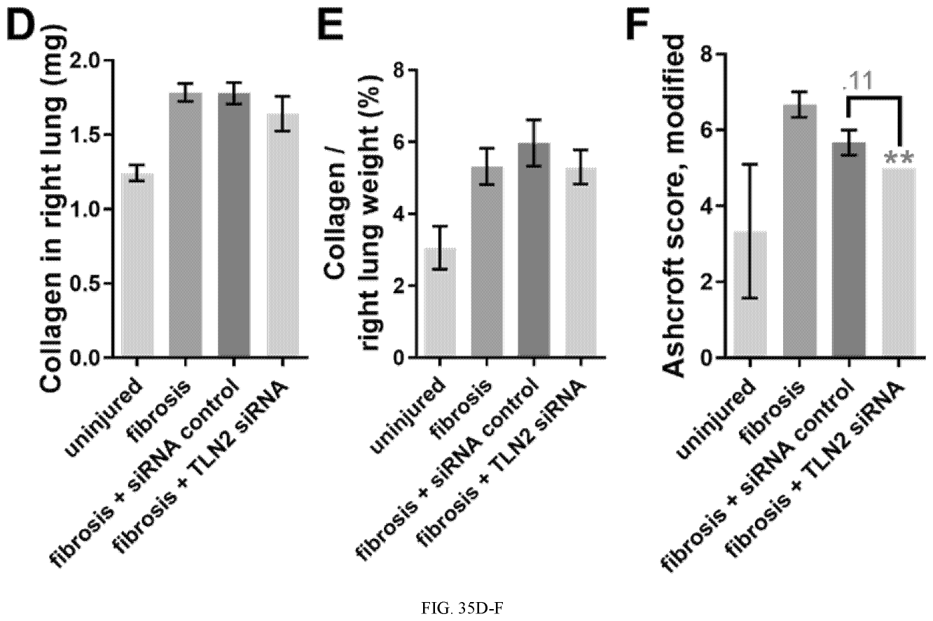

To determine if treatment with talin2 siRNA improved lung fibrosis in both male and female mice, the hydroxyproline-based collagen data (FIGS. 35A, 35B, 35D, and 35E) and Ashcroft-based qualitative data (FIGS. 35C and 35F) was analyzed. The talin2 siRNA treatment improved collagen and Ashcroft readouts in male mice (FIG. 35A-C), but only in the Ashcroft scoring for the female mice (FIG. 3D-F), though this is only with n=3 mice per group.

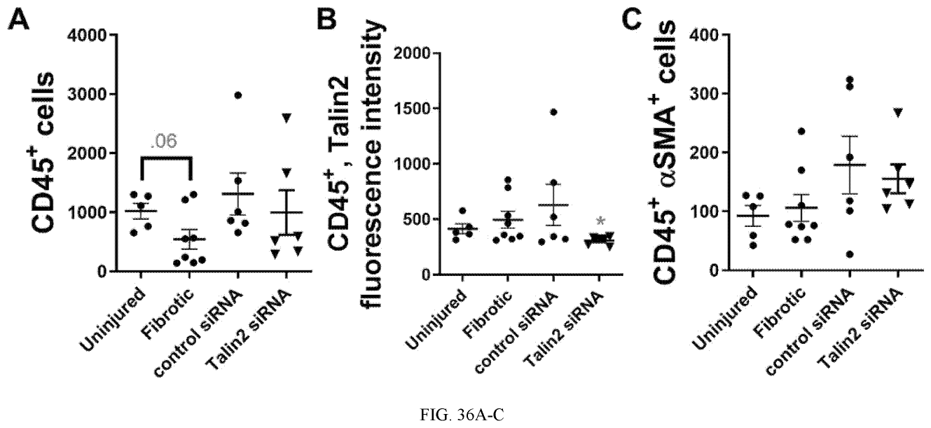

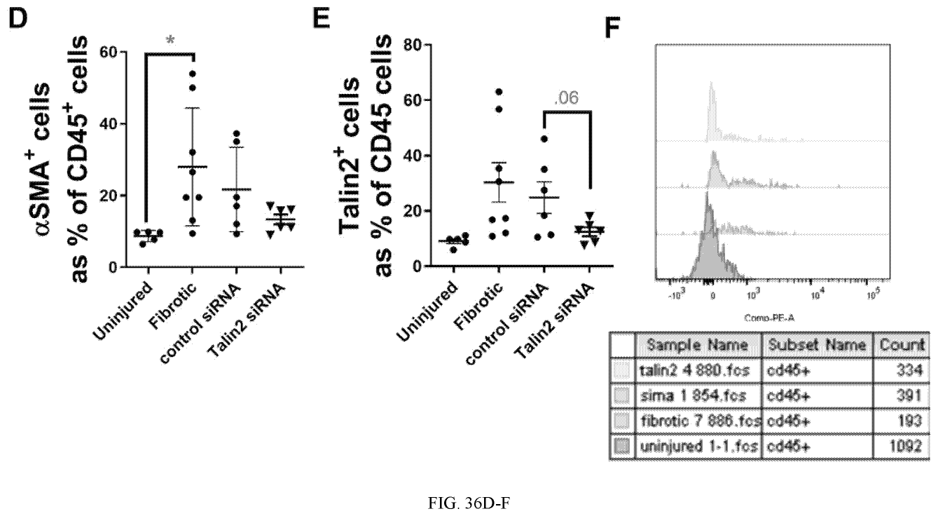

Comparing the broncheo-alveolar lavage (BAL) of fibrotic and talin2 siRNA treated mouse lungs, there was reduced talin2 staining intensity in CD45+ cells, a reduced number of CD45⁺ talin2⁺ double positive cells, and a reduced number of CD45⁺ αSMA⁺ double positive cells (FIG. 36).

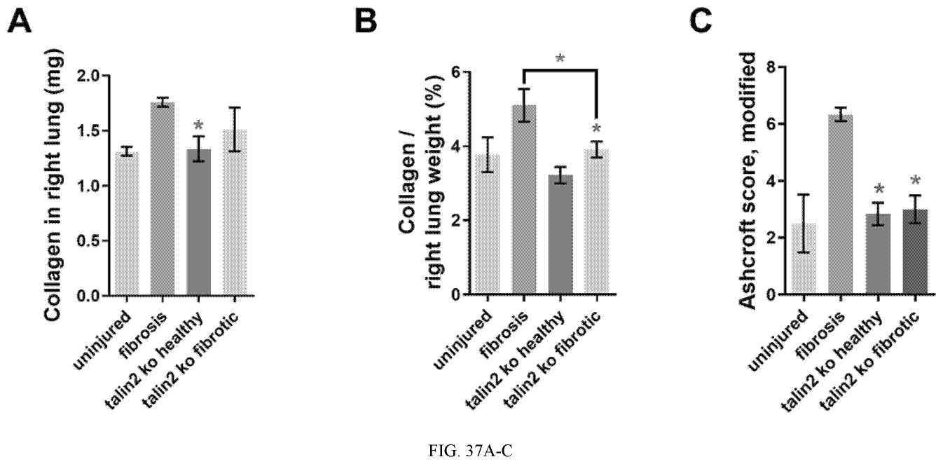

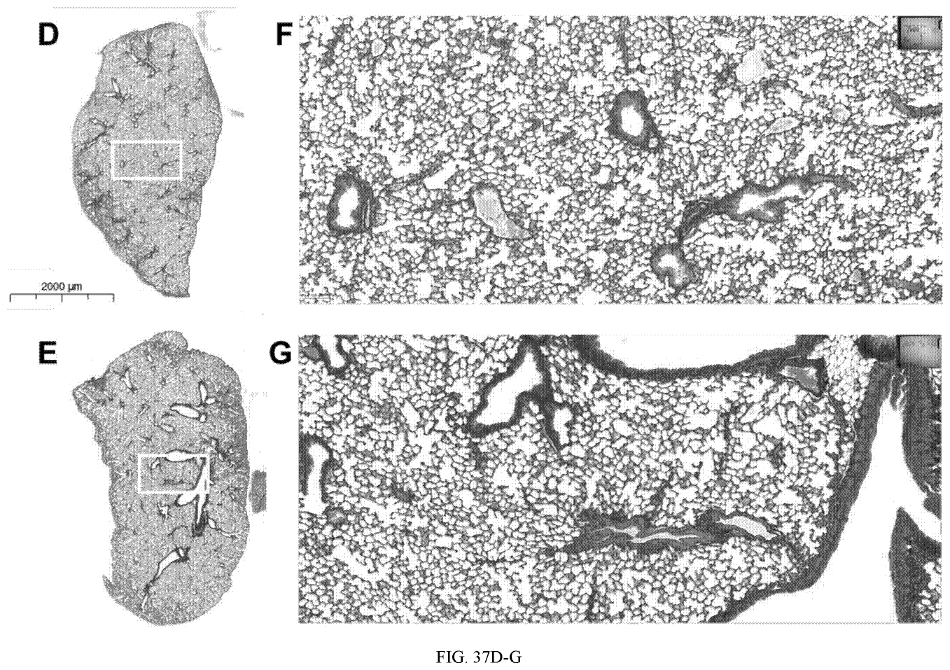

To determine how well talin2 siRNA treatment of fibrotic lungs compared with a complete reduction of talin2, the lungs of Tln2−/− mice were insulted with bleomycin. Tln2−/− lungs were resistant to fibrosis, showing reduced collagen deposition and a significantly improved Ashcroft score (FIG. 37).

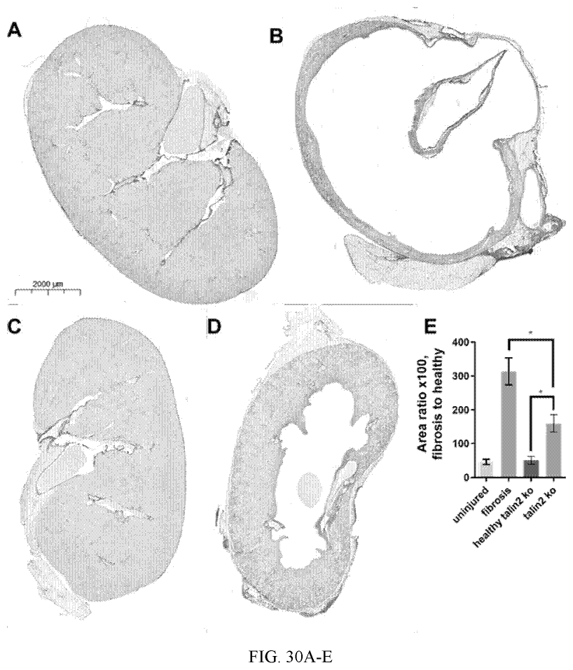

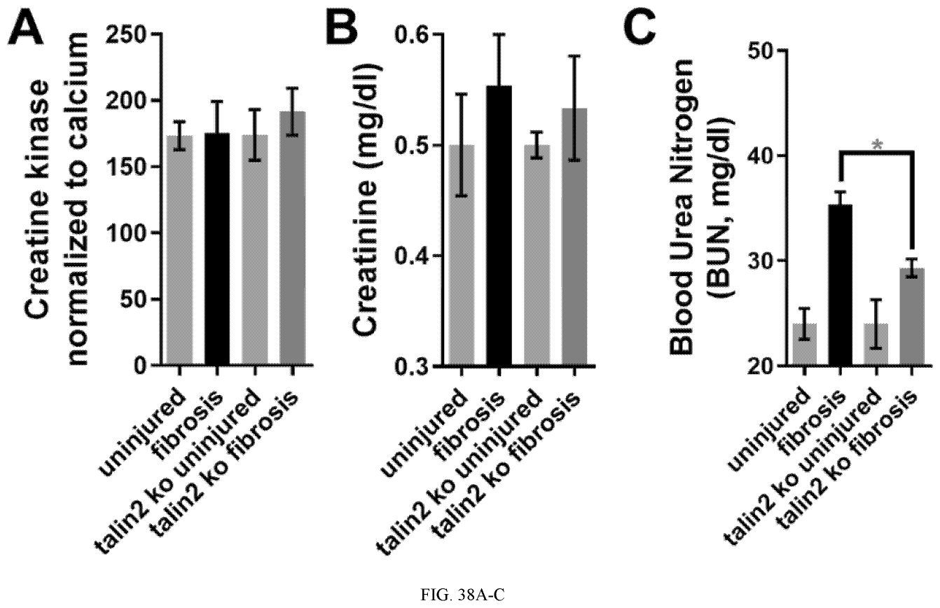

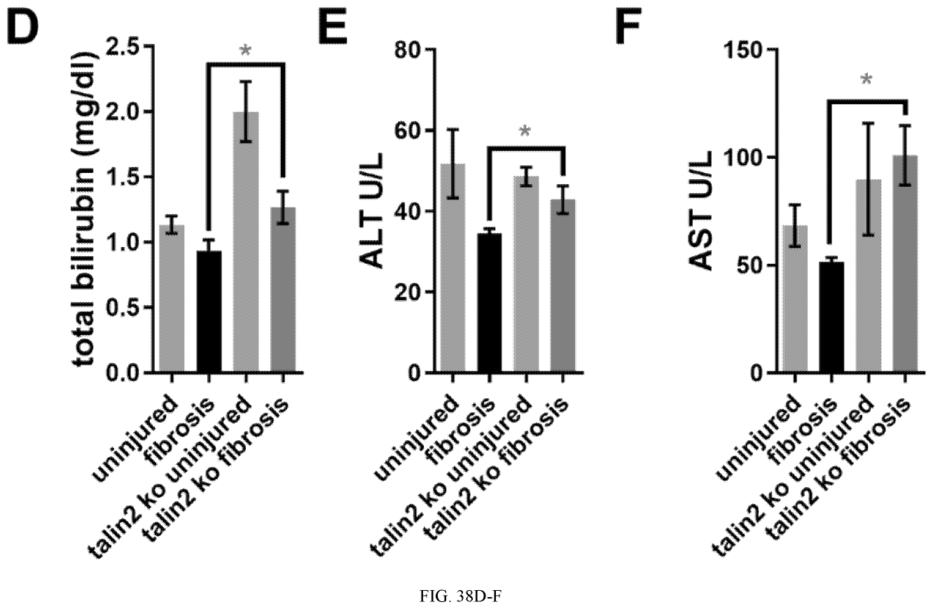

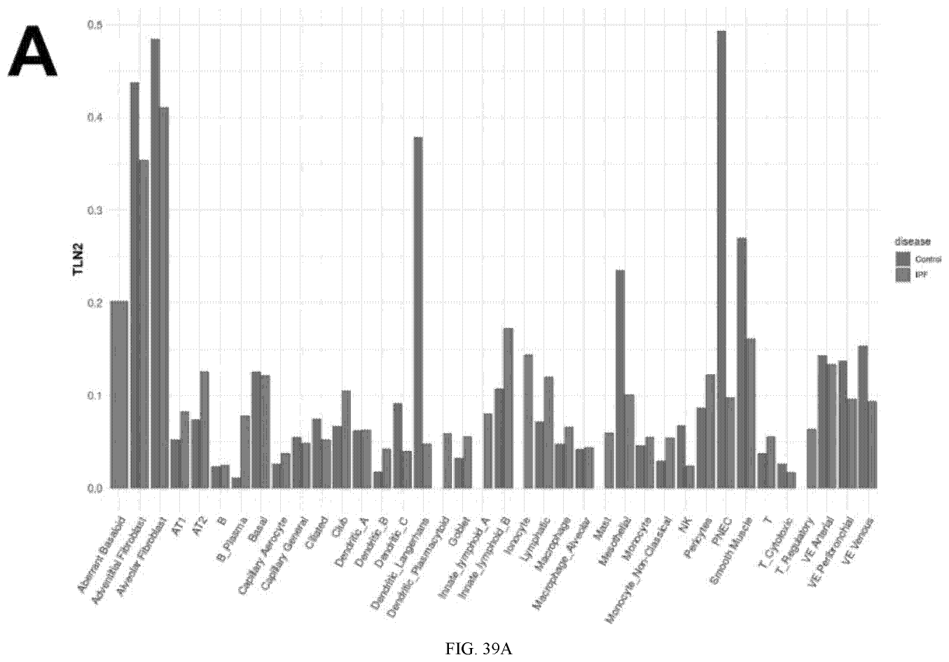

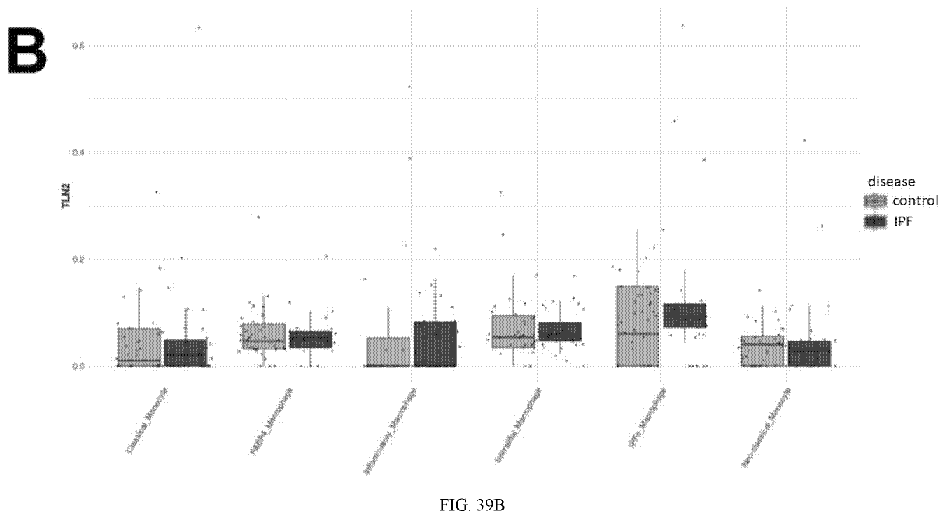

To determine if talin2 contributes to kidney fibrosis as well as lung fibrosis, a unilateral ureteral obstruction (UUO) of the left kidney in Tln2−/− mice was performed, the kidneys were allowed to become fibrotic for 14 days, the mice were sacrificed, and the kidneys were resected. Tln2−/− mice are in the C57B16 background, as such C57B16 mice as utilized controls. The UUO-injured left C57B16 kidney (FIG. 30B) showed significant fibrosis and damage, which was much reduced in the UUO-injured left Tln2−/− kidney (FIG. 30D). The overall amount of collagen-I positive IHC stained tissue (brown) was much reduced in the Tln2−/− kidney (FIG. 30E), and compared favorably to targeted anti-integrin treatments of kidney fibrosis (see e.g., [47] and/or Example 1). Tln2−/− mice that have undergone UUO also have decreased blood urea nitrogen (BUN) and increased bilirubin levels compared to UUO-injured C57B16 mice (FIG. 38). UUO-injured Tln2−/− mice also have higher alanine transferase (ALT) and aspartate transferase (AST) levels than UUO-injured C57B16 mice (FIG. 38E-F). ALT and AST are commonly used markers of liver damage, but kidney fibrosis can counterintuitively cause ALT and AST levels to drop [60]. Several variables showed no significant change, including albumin, amylase, calcium, creatinine, creatine kinase, and uric acid.

### C. Discussion

Herein it is shown that adhesion mechanics play a key role in myofibroblast differentiation (e.g., from monocytes and fibroblasts) and myofibroblast stability, and the utility of modulating adhesion machinery to propose a novel treatment for reversing fibrosis has been shown. The results indicated that culture on somewhat stiffer (e.g., 12 kPa) surfaces induced the expression of talin2, a key mechanosensing and mechanotransduction component of the cytoskeleton, compared to 1 kPa surfaces, which do not support monocyte-myofibroblast differentiation. Inhibition of talin2 can even de-differentiate myofibroblasts, changing their morphology, lowering the amount of αSMA and collagen I produced by myofibroblasts, and changing their secretion profile from pro- to anti-fibrotic. Inhibition of talin2 reversed established lung fibrosis in a murine model, and Tln2−/− mice were resistant to bleomycin-induced lung fibrosis and UUO-induced kidney fibrosis.

To the inventors knowledge, the only method of targeting talin other than siRNA is the small molecule KCH-1521, which decreases adhesion-dependent angiogenesis. KCH-1521, however, does not distinguish between talin1 and talin2 [83]. By contrast, siRNA treatments permit a distinction between talin2 and talin1. Fortunately, Accell siRNA offers promise for delivering talin2 knockdown to the lungs, and it could potentially even be delivered as a dry powder by inhalation [84]. The doses described herein are a factor of 10- to 100-fold lower than have been used in a previous pulmonary study using Accell siRNA [85].

### D. Materials and Methods Study Design

### 1. Study Design

This study examined two related hypotheses. The first is that myofibroblast differentiation (from monocytes and fibroblasts) is governed by adhesion mechanics, and the second is that modulation of adhesion mechanics machinery can be used to guide novel treatment discovery for fibrosing diseases. Specifically, the inventors tested whether inhibition of the intracellular tension sensor talin2 could be used to treat lung fibrosis in mice. Group size was selected based on experience with the pulmonary fibrosis model, particularly based on a pilot experiment using talin2 knockdown in lung fibrosis. Mice were randomized into treatment groups within a cage to eliminate cage effects from the experiment. Treatment was performed by multiple researchers over the course of this study, to ensure reproducibility. Lungs were also resected by multiple researchers, and blinded scoring was used on the fibrosis histology images.

### 2. Purification of Human Monocytes

In order to isolate more peripheral blood mononuclear cells (PBMC) than is possible through a single blood donation, PBMCs were purified from leukocyte reduction filters obtained from the University of Chicago blood donation center, in accordance with human subject protocol at the University of Chicago. All leukocyte reduction filters were de-identified and taken from random blood donors regardless of age, race, or gender. Blood was filtered, and leukocytes purified, the same afternoon as a morning blood donation, to reduce the amount of time that PBMCs could adhere to the filter.

The leukocyte reduction filter was sterilized with 70% ethanol, and the blood flow tubes on either end were clamped to prevent flow. The tube through which filtered blood had exited the filter was cut below the clamp, and a syringe containing 60 ml phosphate buffered saline (PBS) was inserted into the tube. Following this, the tube through which unfiltered blood had entered the filter was unclamped, and PBS was slowly pushed through the filter in the opposite direction of the original blood flow. This direction of flow provided the highest recovery of PBMC, approximately 300 million cells per filter.

The collected blood from the filter was then layered with lymphocyte separation media (LSM), and centrifuged at 1300×G for 20 min. The PBMC layer was then removed by pipetting. These PBMCs were further purified by use of a negative selection kit for human monocytes (Stemcell, Cambridge, MA), per the manufacturer's instructions. The expected yield from each filter was approximately 20 million monocytes. Monocytes were then washed by PBS using five successive 300×G centrifugation steps, in order to remove EDTA from the resulting population. Monocytes were checked for purity using flow cytometry, and average purity was above 95%. Monocytes were cultured immediately following purification, at 100,000 monocytes/cm².

### 3. Purification of Mouse Monocytes

All PBS, plasticware, filters, glassware and magnets were pre-chilled to 4 C, and kept cold throughout this procedure, to limit the clumping of cells. Importantly, ACK lysis buffer caused cell death, and so was not used in this procedure.

Spleens were resected from healthy C57BL/6 mice, pooled, and were placed in PBS with 1 mM EDTA, 2% fetal calf serum (FCS) to prevent subsequent clumping of cells. Spleens were pushed through a 100 mm filter to disassociate the cells. Monocytes were purified from disassociated cells by use of a negative selection kit (Stemcell), following the manufacturer's instructions. The purified monocytes were then washed by PBS using five successive 300×G centrifugation steps, in order to remove EDTA from the resulting population. Monocytes were checked for purity using flow cytometry, and average purity was above 95%. The average yield was 1.5 million monocytes per spleen.

Monocytes were cultured immediately following purification, at 250,000 monocytes/cm².

### 4. Culture of Human and Mouse Monocytes

Human and mouse monocytes were cultured as previously described, using serum-free media (SFM) [86]. Briefly, SFM for human cells is composed of fibrolife (Lifeline, Frederick, MD), with 1× ITS-3 (Sigma, St. Louis, MO), 1× HEPES buffer (Sigma), 1× non-essential amino acids (Sigma), 1× sodium pyruvate (Sigma), and penicillin-streptomycin with glutamate (Sigma). For mice, 2× concentrations of ITS-3, HEPES buffer, non-essential amino acids, and sodium pyruvate are added. For mouse monocytes, 50 mM beta-mercaptoenthanol (ThermoFisher) was also added, as were pro-fibrotic supplements M-CSF (25 ng/ml, Peprotech, Rocky Hill, NJ) and IL-13 (50 ng/ml). Additionally, M-CSF and IL-13 were refreshed in the media of mouse monocytes after 3 days of culture.

Addition of tryptase (Fitzgerald, Acton, MA) to media was as previously described [87]. Monocytes were allowed to differentiate for 5 days, and counted based on morphology as previously described [88].

### 5. Culture of Human and Mouse Fibroblasts

Human fibroblasts (MRC-5, ATCC, Manassas, VA) and mouse fibroblasts (NIH-3T3, ATCC) were cultured in SFM composed for human cells, with 1× concentrations of additives. TGF-b (Peprotech) was added to induce myofibroblast formation at 5 ng/ml [89]. Cells were cultured at 10,000/cm², and TGF-β was refreshed in cultures weekly to maintain the myofibroblast phenotype.

### 6. Methods for Rolling Monocytes in the Presence of Pro-Fibrotic Factors

100,000 human monocytes were suspended in SFM, and placed onto tissue culture treated plastic, or into low adhesion microcentrifuge tubes (protein lo-bind, ThermoFisher). Tryptase was added at 12.5 ng/ml [87], while IL-13 was added at 50 ng/ml. Monocytes were incubated for 1 hr, either adhered to the tissue-culture treated plastic or rotating in low adhesion microcentrifuge tubes. After 1 hr, the adhered monocytes and rotating monocytes were gently washed with four successive PBS washes to remove both tryptase and IL-13 from the surface of the monocytes. The washed monocytes (including those from low adhesion microcentrifuge tubes) were then cultured on tissue culture treated plastic for 5 days, and the number of myofibroblasts was observed through counting of spindle shaped morphological cells and through analysis of alpha-smooth muscle actin positive (αSMA⁺) and collagen I⁺ cells. αSMA and collagen I are widely used markers of myofibroblast differentiation [90].

### 7. Preparation of Low Surface Stiffness Plates

Low stiffness tissue-culture plates were ordered from Matrigen (San Diego, CA). Culture of myofibroblasts on low stiffness surfaces was similar to the culture conditions on tissue-culture treated plasticware. However, low stiffness surfaces were coated in 10 mg/ml fibronectin (Millipore Sigma) for 1 hr at 37 C. Unbound fibronectin was gently removed with 3 successive PBS changes. For experiments involving the comparison of infinite binding surfaces to softer surfaces, the tissue culture treated plasticware was also fibronectin coated.

8. mRNA Purification and RNAseq

mRNA was purified using the Trizol method [91]. RNA sequencing was performed by the University of Chicago Center for Research Informatics, and full protocols for RNA sequencing are available upon request.

9. siRNA Treatment of Human and Mouse Cells

Monocytes are difficult to transfect [92]. As monocytes are non-proliferating cells [92], plasmid-based siRNA platforms that require entry into the nucleus are also unlikely to inhibit protein production across the entire population.

Accell Silencing RNA (siRNA, Dharmacon, Lafayette, CO) is specifically engineered to penetrate cells without the use of transfection reagents, due to a cholesterol modification at one terminus of the sequence. Additionally, Accell siRNA is well suited for a study for silencing proteins in otherwise non-proliferating cells, because each non-replicating siRNA molecule does not have to enter the nucleus, a necessary prerequisite for plasmid-based siRNA platforms. Thus, Accell siRNA targets all cells in a population, not just those that are replicating. Further, that the Accell siRNA does not replicate inside a cell is ideal from a dose-recovery standpoint. Lastly, Accell siRNA contains a modification that inhibits enzymatic digestion within cells. Thus, Accell siRNA made a natural choice for this experiment, and all siRNAs used in this study are Accell siRNA. siRNA was resuspended in RNAse-free ultrapure water, as per the manufacturer's instructions.

The SMARTpool of siRNA targeting human talin2 contained four target sequences, while the anti-parallel corresponding siRNA sequences actually silenced the mRNA. Target sequences: #1 (CCAGAAAACUGAACGAUUA) (SEQ ID NO:29), #2 (GCCCUGUCCUUAAAGAUUU) (SEQ ID NO:30), #3 (CGACUGUGGUUAAAUACUC) (SEQ ID NO:31), and #4 (CGAGAAAGCUUGUGAGUUU) (SEQ ID NO:32). SMARTpool targeting mouse talin2 also contained four target sequences: #1 (CGACUGUGGUUAAAUACUC) (SEQ ID NO:31), #2 (CUGGAAAAUUCAGUGAUGA) (SEQ ID NO:26), #3 (CCCUGGAUUUUGAAGAACA) (SEQ ID NO:27), and #4 (CCAUCGAGUACAUAAAACA) (SEQ ID NO:28). Control non-targeting SMARTpools contained 4 target sequences, including: #1 (CCAGAAAACUGAACGAUUA) (SEQ ID NO:29), #2 (GCCCUGUCCUUAAAGAUUU) (SEQ ID NO:30), #3 (CGACUGUGGUUAAAUACUC) (SEQ ID NO:31), and #4 (CGAGAAAGCUUGUGAGUUU) (SEQ ID NO:32). Individual siRNA tested in mice was talin2 siRNA #2, target: 5′ CUGGAAAAUUCAGUGAUGA 3′ (SEQ ID NO:26), anti-sense: 5′ UCAUCACUGAAUUUUCCAG 3′ (SEQ ID NO:37). Control non-targeting siRNA used in the mouse in vivo study was (CCAGAAAACUGAACGAUUA) (SEQ ID NO:29).

### 10. Flow Cytometry

Myofibroblasts were removed from their tissue culture surfaces by the use of cold trypsin-EDTA (Sigma), followed by mechanical agitation by a rubber policeman. Cells were fixed and permeabilized using Cytofix/Cytoperm (BD biosciences, Franklin Lakes, NJ). Live dead stain was live-dead aqua, used per manufacturer's instructions (ThermoFisher). Compensation was performed via UltraComp beads (ThermoFisher) per the manufacturer's instructions. Antibodies used were anti-collagen I (Biolegend, San Diego, CA), anti-alpha smooth muscle actin (αSMA) (R and D systems, Minneapolis, MN), anti ki-67 (BD biosciences), and anti-talin2 (R and D systems).

11. siRNA Penetration into Cells

siRNA complexed to AF-488 (Dharmacon) was added to human and mouse monocytes and fibroblasts at 50 nM, to determine if the siRNA was penetrating the cell and remaining for up to 2 days. Cells were removed from their culture surface as previously indicated, fixed, and analyzed via flow cytometry.

### 12. Mouse Lung Instillation

All mouse experiments were performed under supervision with protocols approved by the University of Chicago IACUC. Male and female mice were acquired at 8 weeks of age (Jackson laboratories, Bar Harbor, ME) with the intent to be used at 12 weeks of age. However, due to delays regarding COVID19 lockdown and the allowed resumption of non-COVID19 research, the mice were 32 weeks old when the study progressed. Mouse lungs were instilled with bleomycin (0.075 units, 75 ug, Fresenius Kabi, Switzerland) and siRNA (0.2 uM), suspended in endotoxin-free PBS, as previously described [93]. First, mice were anesthetized via isoflurane inhalation (2%). Mice were then placed upright on an angled surface, their tongue pulled to the side, and a 200 ml narrow pipet was placed at the entrance of their throat. 50 ml of PBS was dispensed to the entrance of the throat, and mice were allowed to inhale. Administration to the lungs was confirmed by listening to the mouse's breathing for popping noises. Mice were then weighed and placed on a heating pad to recuperate.

Following bleomycin insult, mice were treated with siRNA using an identical installation procedure. The dose schedule was 7, 9, 11, 14, 16, and 18 days following bleomycin insult. Mice were euthanized at 21 days post insult via injecting of euthasol (Covetrus, Portland, ME) instead of CO2 inhalation, which could damage the lungs.

### 13. Lung Resection and Fibrosis Scoring

Lungs were resected, and perfused with 5 ml of PBS via cardiac puncture. BAL involved exposing the trachea and penetrating the trachea using an 18 gauge needle. The BAL was performed by inserting a catheter needle (Exel International, Redondo Beach, CA) into the trachea, and slowly moving 800 ml of PBS into and out of the lungs. If blood entered the lavage it was discarded. In a previous pilot study testing the efficacy of talin2 siRNA treatment, the lungs were then broncheo-alveolar lavaged (BAL), and the resulting lavage frozen in 10% DMSO. Due to limitations associated with COVID19 scheduling, BAL was not possible in the larger study. This lavage was thawed, fixed, immunostained, and analyzed via flow cytometry to provide the data on talin2's in vivo reduction following siRNA treatment.

After resection, the right and left lobes were separated. The left lobe was fixed in 4% paraformaldehyde overnight, mounted in paraffin, sectioned into 5 mm slices, and stained using Masson's trichrome. Stained lungs were scanned at high resolution using a CRi Panoramic SCAN 40× Whole Slide Scanner (Perkin-Elmer, Waltham, MA), and were read for fibrosis using a modified Ashcroft method, as previously described [59].

The right lobe of the lung was frozen, and dehydrated using a tissue lyophilizer (Labconco, Kansas City, MO). This dehydrated lung was weighed, and was assessed for collagen content by hydroxyproline assay, as previously described [94]. Briefly, dried lungs were digested in 6N HCU/PBS at 100° C. for 24 hr. The supernatant from this digestion was added to 96 well plates and treated sequentially with chloramine-T solution and Ehrlich's solution at 65° C. for 15 min to facilitate the color change reaction. Color was read at 561 nm. Quantification was provided by use of a hydroxyproline (Sigma) dilution series, which was transformed into a standard curve.

### 14. Tln2−/− Mice

Tln2−/− mice (MGI 1917799) were the kind gift of Drs. Roy Zent and David Critchley. Mouse genomic DNA was isolated through ear punches, and Tln2−/− mice were validated genomic DNA by PCR (Transnetyx).

### 15. Kidney Unilateral Ureteral Obstruction (UUO) Fibrosis Model

UUO surgery was performed as previously described [95], with adjustments. Briefly, Mice were anesthetized via 2% isoflurane inhalation, and injected with meloxicam (1 mg/kg), buprenorphine (0.1 mg/kg) in a saline solution, subcutaneously. Briefly, mice were laid on their right side and an abdominal incision used to visualize the left ureter. The left ureter was ligated in the middle section of the ureter with two ties (2 mm apart) using 7-0 silk sutures. Peritoneum is then closed with 5-0 vicryl and skin is closed with 5-0 nylon. 14 days following the UUO ligation, the mice were sacrificed and the kidneys resected. In each case, the UUO ligation was still in place, and in each case it was.

### 16. Assessment of Fibrosis in Kidneys

Right and left kidneys were placed in 4% PFA for 24 hours, mounted in paraffin, sectioned into 5 mm full kidney slices, and stained using immunohistochemistry for collagen I (1:4000, polyclonal rabbit, lifespan biosciences, Seattle WA) via a Bond-Max autostaining system (Leica biosystems, Lincolnshire, IL). Stained kidneys were scanned at high resolution using a CRi Panoramic SCAN 40× Whole Slide Scanner (Perkin-Elmer).

Images were equalized in size, and converted to .tif files using CaseViewer. Images were then imported into imageJ, scale set for conversion between microns and pixels, and deconvoluted with the “H DAB” deconvolution option. The blue stain was thresholded at 215 to see how many pixels were positive for nuclear staining but negative for collagen I, and the brown (IHC positive) image thresholded at 185 to see how many pixels were positive for collagen I. Machine-staining allowed these kidneys to be compared with high reproducibility.

### 17. Blood Analysis for Markers of Kidney Damage

At the time of euthanasia, blood was collected via submandibular bleed into protein low-bind tubes and allowed to coagulate for 2 hours on ice. Coagulated blood was then centrifuged at 10,000×G for 10 min, and serum collected. Serum was then diluted 4× in MilliQ water before being placed on deck on an Alfa Wassermann VetAxcel Blood Chemistry Analyzer. All tests requiring calibration were calibrated on the day of analysis and quality controls were run before analyzing samples. Serum tests were run according to kit instructions, and Creatinine Kinase was normalized to calcium ion concentrations where indicated to account for sample hemolysis.

### 18. Immunofluorescence

Human monocytes from 3 donors were cultured in 8-well chamber slides (Millipore-Sigma) in SFM, and allowed to become myofibroblasts over 3 days. Myofibroblasts were then treated with SMARTpools for non-targeting control siRNA and siRNA targeting human talin2. After 1 week of de-differentiation, the slides were dried quickly using the airflow from a laminar flow hood, in order to preserve cellular morphology as accurately as possible. Cells were then fixed with ice cold 4% PFA, permeabilized with saponin (Sigma). Primary antibodies (anti-talin2, novus) were added at 5 mg/ml overnight. Cells were washed 3× in PBS, and were exposed to DAPI and F-actin-phalloidin-488 (ThermoFisher) for 1 min. Cells were mounted using water-based mowiol mounting media (Southern Biotech, Birmingham, AL) to preserve fluorescence. Slides were imaged immediately using a confocal microscope (Olympus, Shinjuku City, Tokyo).

### 19. Legendplex ELISA

Supernatant from experiments involving culturing human and mouse monocytes and fibroblasts into myofibroblasts on surfaces of different stiffnesses and treatment with siRNA were thawed at 4 C, and centrifuged at 4c and 10,000×G force to pellet cell debris. Supernatant was taken and added to plates, in triplicate. Legendplex (Biolegend) beads against general inflammation markers were added, according to the manufacturer's instructions. Sample readouts were normalized to each individual experiment and donor control.

### 20. Statistical Analysis

Statistical analyses were performed using GraphPad Prism software, and P<0.05 was considered statistically significant. 2-way ANOVA and Student's t-test were used to compare groups.

### E. Abbreviations

alpha-smooth muscle actin (αSMA); Alanine Transferase (ALT); Aspartate Transferase (AST); broncheo-alveolar lavaged (BAL); blood urea nitrogen (BUN); fetal calf serum (FCS); focal adhesions (FAs); Fibrillar adhesions (FBs); idiopathic pulmonary fibrosis (IPF); Immunohistochemistry (IHC); Human fibroblasts (MRC-5); kPa (kilopascals); macrophage-chemotactic protein-1 (MCP1); mouse fibroblasts (NIH-3T3); peripheral blood mononuclear cells (PBMC); phosphate buffered saline (PBS); serum-free media (SFM); Silencing RNA (siRNA); Transforming growth factor β (TGFβ); Talin2 (Tln2); Tumor necrosis factor α (TNFα); Unilateral Ureteral Obstruction (UUO).

### F. References

The following references and those listed throughout the patent application, to the extent that they provide exemplary procedural or other details supplementary to those set forth herein, are specifically incorporated herein by reference.
1. Wynn, T. A., Cellular and molecular mechanisms of fibrosis. J Pathol, 2008. 214(2): p. 199-210.
2. Wynn, T. A., Fibrotic disease and the T(H)1/T(H)2 paradigm. Nat Rev Immunol, 2004. 4(8): p. 583-94.
3. Spagnolo, P., Novel treatments for idiopathic pulmonary fibrosis. Am J Med, 2015.
4. Xaubet, A., A. Serrano-Mollar, and J. Ancochea, Pirfenidone for the treatment of idiopathic pulmonary fibrosis. Expert Opin Pharmacother, 2014. 15(2): p. 275-81.
5. Knuppel, L., et al., A Novel Antifibrotic Mechanism of Nintedanib and Pirfenidone. Inhibition of Collagen Fibril Assembly. Am J Respir Cell Mol Biol, 2017. 57(1): p. 77-90.
6. Raghu, G., et al., Long-term treatment with recombinant human pentraxin 2 protein inpatients with idiopathic pulmonary fibrosis: an open-label extension study. Lancet Respir Med, 2019. 7(8): p. 657-664.
7. Madsen, D. H., et al., M2-like macrophages are responsible for collagen degradation through a mannose receptor-mediated pathway. J Cell Biol, 2013. 202(6): p. 951-66.
8. Hinz, B. and G. Gabbiani, Fibrosis: recent advances in myofibroblast biology and new therapeutic perspectives. F1000 Biol Rep, 2010. 2: p. 78.
9. Hinz, B., Mechanical aspects of lung fibrosis: a spotlight on the myofibroblast. Proc Am Thorac Soc, 2012. 9(3): p. 137-47.
10. Klingberg, F., B. Hinz, and E. S. White, The myofibroblast matrix: implications for tissue repair and fibrosis. J Pathol, 2013. 229(2): p. 298-309.
11. Hinz, B., Formation and function of the myofibroblast during tissue repair. J Invest Dermatol, 2007. 127(3): p. 526-37.
12. Liu, F., et al., Feedback amplification of fibrosis through matrix stiffening and COX-2 suppression. J Cell Biol, 2010. 190(4): p. 693-706.
13. Tomasek, J. J., et al., Myofibroblasts and mechano-regulation of connective tissue remodelling. Nat Rev Mol Cell Biol, 2002. 3(5): p. 349-63.
14. Abe, R., et al., Peripheral blood fibrocytes: differentiation pathway and migration to wound sites. J Immunol, 2001. 166(12): p. 7556-62.
15. Forbes, S. J., et al., A significant proportion of myofibroblasts are of bone marrow origin in human liver fibrosis. Gastroenterology, 2004. 126(4): p. 955-63.
16. Direkze, N. C., et al., Multiple organ engraftment by bone-marrow-derived myofibroblasts and fibroblasts in bone-marrow-transplanted mice. Stem Cells, 2003. 21(5): p. 514-20.
17. Hashimoto, N., et al., Bone marrow-derived progenitor cells in pulmonary fibrosis. J Clin Invest, 2004. 113(2): p. 243-52.
18. Nakashima, T., et al., Lung bone marrow-derived hematopoietic progenitor cells enhance pulmonary fibrosis. Am J Respir Crit Care Med, 2013. 188(8): p. 976-84.
19. Mori, L., et al., Fibrocytes contribute to the myofibroblast population in wounded skin and originate from the bone marrow. Exp Cell Res, 2005. 304(1): p. 81-90.
20. Lagares, D., et al., Targeted apoptosis of myofibroblasts with the BH3 mimetic ABT-263 reverses established fibrosis. Sci Transl Med, 2017. 9(420).
21. Balestrini, J. L., et al., The mechanical memory of lung myofibroblasts. Integr Biol (Camb), 2012. 4(4): p. 410-21.
22. Hoffman, B. D., C. Grashoff, and M. A. Schwartz, Dynamic molecular processes mediate cellular mechanotransduction. Nature, 2011. 475(7356): p. 316-23.
23. Geiger, B., J. P. Spatz, and A. D. Bershadsky, Environmental sensing through focal adhesions. Nat Rev Mol Cell Biol, 2009. 10(1): p. 21-33.
24. Roca-Cusachs, P., et al., Clustering of alpha(5)beta(1) integrins determines adhesion strength whereas alpha(v)beta(3) and talin enable mechanotransduction. Proc Natl Acad Sci USA, 2009. 106(38): p. 16245-50.
25. Dugina, V., et al., Focal adhesion features during myofibroblastic differentiation are controlled by intracellular and extracellular factors. J Cell Sci, 2001. 114(Pt 18): p. 3285-96.
26. Praekelt, U., et al., New isoform-specific monoclonal antibodies reveal different sub-cellular localisations for talin1 and talin2. Eur J Cell Biol, 2012. 91(3): p. 180-91.
27. Morton, P. E. and M. Parsons, Dissecting cell adhesion architecture using advanced imaging techniques. Cell Adh Migr, 2011. 5(4): p. 351-9.
28. Debrand, E., et al., Mice carrying a complete deletion of the talin2 coding sequence are viable and fertile. Biochem Biophys Res Commun, 2012. 426(2): p. 190-5.
29. Anthis, N. J., et al., Structural diversity in integrin talin interactions. Structure, 2010. 18(12): p. 1654-66.
30. Critchley, D. R., Biochemical and structural properties of the integrin-associated cytoskeletal protein talin. Annu Rev Biophys, 2009. 38: p. 235-54.
31. Debrand, E., et al., Talin 2 is a large and complex gene encoding multiple transcripts and protein isoforms. FEBS J, 2009. 276(6): p. 1610-28.
32. Manso, A. M., et al., Talin1 has unique expression versus talin 2 in the heart and modifies the hypertrophic response to pressure overload. J Biol Chem, 2013. 288(6): p. 4252-64.
33. Gough, R. E. and B. T. Goult, The tale of two talins—two isoforms to fine-tune integrin signalling. FEBS Lett, 2018. 592(12): p. 2108-2125.
34. Anthis, N. J., et al., The structure of an integrin/talin complex reveals the basis of inside-out signal transduction. EMBO J, 2009. 28(22): p. 3623-32.
35. Austen, K., et al., Extracellular rigidity sensing by talin isoform-specific mechanical linkages. Nat Cell Biol, 2015. 17(12): p. 1597-606.
36. Haining, A. W., et al., All Subdomains of the Talin Rod Are Mechanically Vulnerable and May Contribute To Cellular Mechanosensing. ACS Nano, 2016. 10(7): p. 6648-58.
37. Monkley, S. J., et al., Disruption of the talin gene arrests mouse development at the gastrulation stage. Dev Dyn, 2000. 219(4): p. 560-74.
38. Chen, N. T. and S. H. Lo, The N-terminal half of talin2 is sufficient for mouse development and survival. Biochem Biophys Res Commun, 2005. 337(2): p. 670-6.
39. Kanchanawong, P., et al., Nanoscale architecture of integrin-based cell adhesions. Nature, 2010. 468(7323): p. 580-4.
40. del Rio, A., et al., Stretching single talin rod molecules activates vinculin binding. Science, 2009. 323(5914): p. 638-41.
41. Liu, J., et al., Talin determines the nanoscale architecture of focal adhesions. Proc Natl Acad Sci USA, 2015. 112(35): p. E4864-73.
42. Goksoy, E., et al., Structural basis for the autoinhibition of talin in regulating integrin activation. Mol Cell, 2008. 31(1): p. 124-33.
43. Ingmar Schoen, B. L. P., and Viola Vogel, The Yin-Yang of Rigidity Sensing: How Forces and Mechanical Properties Regulate the Cellular Response to Materials. Annual Review of Materials Research, 2013. 43: p. 589-618.
44. Hytonen, V. P. and V. Vogel, How force might activate talin's vinculin binding sites: SMD reveals a structural mechanism. PLoS Comput Biol, 2008. 4(2): p. e24.
45. Wells, R. G., The role of matrix stiffness in regulating cell behavior. Hepatology, 2008. 47(4): p. 1394-400.
46. Handorf, A. M., et al., Tissue stiffness dictates development, homeostasis, and disease progression. Organogenesis, 2015. 11(1): p. 1-15.
47. Michael J V White, Melis Ozkan, Michal M Ra̧czy, Jorge Emiliano Gomez-Medellin, Kyle M Koss, Aaron T. Alpar, Bilal Naved, Jason Wertheim, Jeffrey A Hubbell Blocking antibodies against integrin-α3, integrin-αM, and integrin-αMβ2 de-differentiate myofibroblasts and reverse lung and kidney fibroses in a mouse model. 2021. DOI: https://doi.org/10.1101/2021.06.07.447405
48. Shi, J., et al., Anti-fibrotic actions of interleukin-10 against hypertrophic scarring by activation of PI3K/AKT and STAT3 signaling pathways in scar-forming fibroblasts. PLoS One, 2014. 9(5): p. e98228.
49. Gasse, P., et al., IL-1 and IL-23 mediate early IL-17A production in pulmonary inflammation leading to late fibrosis. PLoS One, 2011. 6(8): p. e23185.
50. Yogo, Y., et al., Macrophage derived chemokine (CCL22), thymus and activation-regulated chemokine (CCL17), and CCR4 in idiopathic pulmonary fibrosis. Respir Res, 2009. 10: p. 80.
51. Le, T. T., et al., Blockade of IL-6 Trans signaling attenuates pulmonary fibrosis. J Immunol, 2014. 193(7): p. 3755-68.
52. Belperio, J. A., et al., The role of the Th2 CC chemokine ligand CCL17 in pulmonary fibrosis. J Immunol, 2004. 173(7): p. 4692-8.
53. Huaux, F., et al., A profibrotic function of IL-12p40 in experimental pulmonary fibrosis. J Immunol, 2002. 169(5): p. 2653-61.
54. Sahin, H. and H. E. Wasmuth, Chemokines in tissue fibrosis. Biochim Biophys Acta, 2013. 1832(7): p. 1041-8.
55. Oikonomou, N., et al., Soluble TNF mediates the transition from pulmonary inflammation to fibrosis. PLoS One, 2006. 1: p. e108.
56. Borthwick, L. A., The IL-1 cytokine family and its role in inflammation and fibrosis in the lung. Semin Immunopathol, 2016. 38(4): p. 517-34.
57. Gallucci, R. M., E. G. Lee, and J. J. Tomasek, IL-6 modulates alpha-smooth muscle actin expression in dermal fibroblasts from IL-6-deficient mice. J Invest Dermatol, 2006. 126(3): p. 561-8.
58. Iyonaga, K., et al., Monocyte chemoattractant protein-1 in idiopathic pulmonary fibrosis and other interstitial lung diseases. Hum Pathol, 1994. 25(5): p. 455-63.
59. Hubner, R. H., et al., Standardized quantification of pulmonary fibrosis in histological samples. Biotechniques, 2008. 44(4): p. 507-11, 514-7.
60. Ray, L., et al., A comparative study of serum aminotransferases in chronic kidney disease with and without end-stage renal disease: Need for new reference ranges. Int J Appl Basic Med Res, 2015. 5(1): p. 31-5.
61. Hinz, B., et al., The myofibroblast: one function, multiple origins. Am J Pathol, 2007. 170(6): p. 1807-16.
62. Pinon, P., et al., Talin-bound NPLY motif recruits integrin-signaling adapters to regulate cell spreading and mechanosensing. J Cell Biol, 2014. 205(2): p. 265-81.
63. Discher, D. E., P. Janmey, and Y. L. Wang, Tissue cells feel and respond to the stiffness of their substrate. Science, 2005. 310(5751): p. 1139-43.
64. Yeung, T., et al., Effects of substrate stiffness on cell morphology, cytoskeletal structure, and adhesion. Cell Motil Cytoskeleton, 2005. 60(1): p. 24-34.
65. Hinz, B., Masters and servants of the force: the role of matrix adhesions in myofibroblast force perception and transmission. Eur J Cell Biol, 2006. 85(3-4): p. 175-81.
66. Goffin, J. M., et al., Focal adhesion size controls tension-dependent recruitment of alpha-smooth muscle actin to stress fibers. J Cell Biol, 2006. 172(2): p. 259-68.
67. Wells, R. G., The role of matrix stiffness in hepatic stellate cell activation and liver fibrosis. J Clin Gastroenterol, 2005. 39(4 Suppl 2): p. S158-61.
68. Tarasova, N. K., et al., Proteomics Reveals a Role for Attachment in Monocyte Differentiation into Efficient Proinflammatory Macrophages. J Proteome Res, 2015. 14(9): p. 3940-7.
69. Lawson, C., et al., FAK promotes recruitment of talin to nascent adhesions to control cell motility. J Cell Biol, 2012. 196(2): p. 223-32.
70. Pilling, D., et al., TNF-alpha-stimulated fibroblasts secrete lumican to promote fibrocyte differentiation. Proc Natl Acad Sci USA, 2015. 112(38): p. 11929-34.
71. Ashley, S. L., et al., Periostin regulates fibrocyte function to promote myofibroblast differentiation and lung fibrosis. Mucosal Immunol, 2017. 10(2): p. 341-351.
72. Pilling, R. G. a. D., Fibrocytes: New Insights into Tissue Repair, in Fibrocytes: New Insights into Tissue Repair, R. Bucala, Editor. 2007, Yale University Press: USA. p. 268.
73. Lim, J., et al., An essential role for talin during alpha(M)beta(2)-mediated phagocytosis. Mol Biol Cell, 2007. 18(3): p. 976-85.
74. Qi, L., et al., Talin2-mediated traction force drives matrix degradation and cell invasion. J Cell Sci, 2016. 129(19): p. 3661-3674.
75. Zaidel-Bar, R., et al., Functional atlas of the integrin adhesome. Nat Cell Biol, 2007. 9(8): p. 858-67.
76. Zhang, X., et al., Talin depletion reveals independence of initial cell spreading from integrin activation and traction. Nat Cell Biol, 2008. 10(9): p. 1062-8.
77. Theodosiou, M., et al., Kindlin-2 cooperates with talin to activate integrins and induces cell spreading by directly binding paxillin. Elife, 2016. 5: p. e10130.
78. Muiznieks, L. D. and F. W. Keeley, Molecular assembly and mechanical properties of the extracellular matrix: A fibrous protein perspective. Biochim Biophys Acta, 2013. 1832(7): p. 866-75.
79. Eckes, B. and T. Krieg, Regulation of connective tissue homeostasis in the skin by mechanical forces. Clin Exp Rheumatol, 2004. 22(3 Suppl 33): p. S73-6.
80. Marenzana, M., et al., The origins and regulation of tissue tension: identification of collagen tension-fixation process in vitro. Exp Cell Res, 2006. 312(4): p. 423-33.
81. Marinkovic, A., F. Liu, and D. J. Tschumperlin, Matrices of physiologic stiffness potently inactivate idiopathic pulmonary fibrosis fibroblasts. Am J Respir Cell Mol Biol, 2013. 48(4): p. 422-30.
82. Neumark, N., et al., The Idiopathic Pulmonary Fibrosis Cell Atlas. Am J Physiol Lung Cell Mol Physiol, 2020. 319(6): p. L887-L893.
83. Lim, I. R., et al., Talin Modulation by a Synthetic N-Acylurea Derivative Reduces Angiogenesis in Human Endothelial Cells. Int J Mol Sci, 2017. 18(1).
84. Thanki, K., et al., Formulation of RNA interference-based drugs for pulmonary delivery: challenges and opportunities. Ther Deliv, 2018. 9(10): p. 731-749.
85. Zafra, M. P., et al., Gene silencing of SOCS3 by siRNA intranasal delivery inhibits asthma phenotype in mice. PLoS One, 2014. 9(3): p. e91996.
86. Pilling, D., V. Vakil, and R. H. Gomer, Improved serum-free culture conditions for the differentiation of human and murine fibrocytes. Journal of immunological methods, 2009. 351(1-2): p. 62-70.
87. White, M. J. and R. H. Gomer, Trypsin, Tryptase, and Thrombin Polarize Macrophages towards a Pro-Fibrotic M2a Phenotype. PLoS One, 2015. 10(9): p. e0138748.
88. Crawford, J. R., D. Pilling, and R. H. Gomer, Improved serum-free culture conditions for spleen-derived murine fibrocytes. J Immunol Methods, 2010. 363(1): p. 9-20.
89. Hinz, B., et al., Myofibroblast development is characterized by specific cell-cell adherens junctions. Mol Biol Cell, 2004. 15(9): p. 4310-20.
90. Baum, J. and H. S. Duffy, Fibroblasts and myofibroblasts: what are we talking about? J Cardiovasc Pharmacol, 2011. 57(4): p. 376-9.
91. Rio, D. C., et al., Purification of RNA using TRIzol (TRI reagent). Cold Spring Harb Protoc, 2010. 2010(6): p. pdb prot5439.
92. Moradian, H., et al., mRNA Transfection-Induced Activation of Primary Human Monocytes and Macrophages: Dependence on Carrier System and Nucleotide Modification. Sci Rep, 2020. 10(1): p. 4181.
93. Katsumata, K., et al., Targeting inflammatory sites through collagen affinity enhances the therapeutic efficacy of anti-inflammatory antibodies. Sci Adv, 2019. 5(11): p. eaay1971.
94. Bergheim, I., et al., Critical role of plasminogen activator inhibitor-1 in cholestatic liver injury and fibrosis. J Pharmacol Exp Ther, 2006. 316(2): p. 592-600.
95. Cochrane, A. L., et al., Renal structural and functional repair in a mouse model of reversal of ureteral obstruction. J Am Soc Nephrol, 2005. 16(12): p. 3623-30.
96. Subramanian, A., et al., Gene set enrichment analysis: a knowledge-based approach for interpreting genome-wide expression profiles. Proc Natl Acad Sci USA, 2005. 102(43): p. 15545-50.

### G. Tables

TABLE 3
Representative human tissue measurements for Young's elastic
modulus. Data derived from [45], and [46]).

| Human tissue | Stiffness (kPa) |
|---|---|
| Brain | 0.2 |
| Skin | 3 |
| Muscle | 12 |
| Heart | 15 |
| Scar tissue | 17 |
| Bone | 50 |

TABLE 4
Upregulated and downregulated RNAseq transcripts.

| Upreg- | | Downreg- | | Investigated | |
|---|---|---|---|---|---|
| ulated | Fold | ulated | Fold | protein | Fold |
| proteins | change | proteins | change | targets | change |
| CCL22 | 12.98527 | IGKV1-5 | −655.835 | TREM1 | −4.13228 |
| KANK3 | 14.68754 | GAL3ST4 | −47.2901 | ITGAM | 1.797671 |
| COL22A1 | 21.01883 | CXCL9 | −16.552 | TLN2 | 2.860702 |
| PRSS3 | 30.94083 | — | — | ITGA3 | 3.536679 |
| VIT | 47.29368 | — | — | — | — |
| RASL12 | 47.58518 | — | — | — | — |
| RNF128 | 98.61058 | — | — | — | — |

TABLE 5
Upregulated and downregulated RNAseq pathways, and functional
annotation clustering. Enrichment score is calculated based
on the maximum deviation from 0 in the pathway analysis [96].
In this analysis, 5.18 is the highest possible score.

| | | Functional | |
|---|---|---|---|
| Upregulated | Downregulated | annotation | Enrichment |
| pathways | pathways | clustering | score |
| Chemokine | Role of nfat in | Complement | 5.18 |
| signaling | regulation of the | activation | |
| | immune response | | |
| Leukocyte | TREM1 | Chemokine | 5.18 |
| extravasation | | signaling | |
| signaling | | | |
| IL-6 | PTEN | Phagocytosis | 5.18 |
| signaling | signaling | recognition | |
| Paxillin | Coagulation | positive regulation | 5.18 |
| signaling | system | of inflammatory | |
| | | response | |
| Integrin | — | monocyte | 5.18 |
| signaling | | chemotaxis | |
| IL-13 | — | receptor- mediated | 5.18 |
| signaling | | endocytosis | |

All of the methods disclosed and claimed herein can be made and executed without undue experimentation in light of the present disclosure. While the compositions and methods of this invention have been described in terms of preferred embodiments and aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A nucleic acid having a sequence that has at least 80% sequence identity to one of SEQ ID NOS:18-25 or 29-36, wherein the nucleic acid comprises a modified nucleic acid.

2-29. (canceled)

30. A host cell comprising the nucleic acid of claim 1.

31-32. (canceled)

33. A composition comprising the nucleic acid of claim 1.

34. (canceled)

35. A method for making a nucleic acid comprising transferring the nucleic acid of claim 1 into a cell and isolating replicated or transcribed nucleic acids.

36. A method for treating and/or reversing fibrosis in a subject comprising administering a composition comprising an inhibitor of Talin2 or the composition of claim 33 to the subject.

37-52. (canceled)

53. An antibody conjugate comprising an integrin α3, αM, or αMβ2 antibody operatively linked to an extracellular matrix (ECM)-affinity peptide.

54-72. (canceled)

73. One or more nucleic acid encoding the antibody conjugate ofany claim 53.

74. An expression vector comprising the nucleic acid of claim 73.

75. A host cell comprising the nucleic acid of claim 73.

76. A method for making an antibody conjugate comprising expressing the one or more nucleic acids of claim 73 in a cell and isolating the expressed protein.

77-78. (canceled)

79. A composition comprising the antibody conjugate of claim 53.

80. A method for treating kidney fibrosis in a subject comprising administering a composition comprising an anti-TGFβ antibody operatively linked to an ECM-affinity peptide.

81. (canceled)

82. A method for treating and/or reversing fibrosis in a subject comprising administering the composition of claim 79 or a composition comprising an inhibitor or blocking agent of integrin α3, αM, αMβ2, or combinations thereof to the subject.

83. (canceled)

84. The method of claim 82, wherein the inhibitor comprises an anti-αMβ2 CBRM1/5 antibody or an anti-α3 3F9G4 antibody.

85. The method of claim 82, wherein the fibrosis comprises dermal, heart, renal, liver, or pulmonary fibrosis.

86-93. (canceled)

94. The method of claim 82, wherein the inhibitor is linked to an extracellular matrix (ECM)-affinity peptide.

95. The method of claim 94, wherein the ECM-affinity peptide comprises a decorin peptide.

96. The method of claim 95, wherein the decorin peptide comprises the amino acid sequence of one of SEQ ID NO:1-3 or comprises a peptide with at least 85% sequence identity to the amino acid sequence of one of SEQ ID NO:1-3.

97-106. (canceled)

107. The method of claim 94, wherein the peptide is covalently linked to the antibody.

108-109. (canceled)

110. The method of claim 94, wherein the ratio of peptide to antibody is about 1:1 to 10:1.
